(19) United States
(12) Patent Application Publication
(10) Pub. No.: US 2010/0055111 A1
(43) Pub. Date: Mar. 4, 2010

(54) INDOLEAMINE 2,3-DIOXYGENASE, PD-1/PD-L PATHWAYS, AND CTLA4 PATHWAYS IN THE ACTIVATION OF REGULATORY T CELLS

(75) Inventors: Madhav D. Sharma, Augusta, GA (US); Bruce R. Blazar, Golden Valley, MN (US); Andrew L. Mellor, Augusta, GA (US); David H. Munn, Augusta, GA (US)

Correspondence Address: MUETING, RAASCH & GEBHARDT, P.A., P.O. BOX 581336, MINNEAPOLIS, MN, 55458-1336, US

(73) Assignees: Med. College of Georgia Research Institute, Inc., Augusta, GA (US); Regents of the University of Minnesota, St. Paul, MN (US)

(21) Appl. No.: 12/526,909

(22) PCT Filed: Feb. 14, 2008
(86) PCT No.: PCT/US08/01946

§ 371 Date: Nov. 12, 2009

Related U.S. Application Data
(60) Provisional application No. 60/901,229, filed on Feb. 14, 2007.
(60) Provisional application No. 60/959,053, filed on Jul. 11, 2007.

(51) Int. Cl. A61K 39/395; A61P 31/00; A61P 37/04; A61P 35/00

(52) U.S. Cl. 424/158.1; 424/184.1; 424/173.1

(57) ABSTRACT

The present invention includes methods of enhancing immune responses by administering an inhibitor of indoleamine-2,3-dioxygenase (IDO) along with one or more inhibitors of the PD-1/PD-L pathway and/or one or more inhibitors of the CTLA4 pathway.

### CONTINUING APPLICATION DATA

This application claims the benefit of U.S. Provisional Application Ser. Nos. 60/901,229, filed Feb. 14, 2007, and 60/959,053, filed Jul. 11, 2007, each of which is incorporated by reference herein.

### GOVERNMENT FUNDING

The present invention was made with government support under Grant Nos. CA103320, CA096651, CA112431, HD41187, AI063402, awarded by the National Institutes of Health. The Government may have certain rights in this invention.

### BACKGROUND

Plasmacytoid dendritic cells (pDCs) are a unique dendritic cell (DC) subset that plays a critical role in regulating innate and adaptive immune responses (Liu, 2005, Annu Rev Immunol; 23:275-306). In addition to stimulating immune responses, increasing evidence suggests that pDC's may also represent a naturally occurring regulatory DC subset (Chen, 2005, Curr Opin Organ Transplant; 10:181-185). Under certain circumstances pDCs appear to be able to induce the differentiation of regulatory T cells (Tregs) that downregulate immune responses (Martin et al., 2002, Blood; 100:383-390). In humans, pDCs can prime allogeneic naive CD8+ T cells to differentiate into CD8+ suppressor T cells (Gilliet and Liu, 2002, J Exp Med; 195:695-704; Wei et al., 2005, Cancer Res; 65:5020-5026). It has recently been shown that human pDCs also induce the generation of CD4+ regulatory T cells (Tregs) (Moseman et al., 2004, J Immunol; 173:4433-4442).

Tregs inhibit autologous or allogeneic T cell proliferation in vitro and are critical in maintaining self-tolerance and controlling excessive immune reactions (Sakaguchi, 2005, Nat Immunol; 6:345-352). There is a need to further the understanding of the mechanisms underlying pDC-induced Treg generation and activation. Such an improved understanding will provide powerful new means for modulating immune responses.

### SUMMARY OF THE INVENTION

The present invention includes a method of enhancing an immune response, the method including administering an inhibitor of indoleamine-2,3-dioxygenase (IDO) and one or more inhibitors of the PD-1/PD-L pathway. In some embodiments, two or more inhibitors of the PD-1/PD-L pathway may be administered, and in some embodiments, the two or more inhibitors of the PD-1/PD-L pathway may be administered in combination, as a cocktail. In some embodiments, one or more inhibitors of the PD-1/PD-L pathway include one or more antibodies against PD-1, PD-L1, and/or PD-L2. In some embodiments, the method further includes the administration of one or more inhibitors of the CTLA4 pathway.

The present invention includes a method to enhance an immune response to an antigen in a subject, the method including administering to the subject an effective amount of such an antigen in combination with an inhibitor of IDO and one or more inhibitors of the PD-1/PD-L pathway.

The present invention includes a method of reducing immune suppression mediated by regulatory T cells (Tregs) in a subject, the method including administering to the subject an inhibitor of indoleamine-2,3-dioxygenase (IDO) and one or more inhibitors of the PD-1/PD-L pathway. In some embodiments, two or more inhibitors of the PD-1/PD-L pathway may be administered, and in some embodiments, the two or more inhibitors of the PD-1/PD-L pathway may be administered in combination, as a cocktail. In some embodiments, one or more inhibitors of the PD-1/PD-L pathway include one or more antibodies against PD-1, PD-L1, and/or PD-L2. In some embodiments, the method further includes the administration of one or more inhibitors of the CTLA4 pathway.

The present invention includes a method of enhancing a T cell mediated immune response, the method including administering the method comprising administering to the subject an inhibitor of indoleamine-2,3-dioxygenase (IDO) and one or more inhibitors of the PD-1/PD-L pathway. In some embodiments, two or more inhibitors of the PD-1/PD-L pathway may be administered, and in some embodiments, the two or more inhibitors of the PD-1/PD-L pathway may be administered in combination, as a cocktail. In some embodiments, one or more inhibitors of the PD-1/PD-L pathway include one or more antibodies against PD-1, PD-L1, and/or PD-L2. In some embodiments, the method further includes the administration of one or more inhibitors of the CTLA4 pathway.

The present invention includes a method of treating cancer in a subject, the method including administering to the subject an inhibitor of indoleamine-2,3-dioxygenase (IDO) and one or more inhibitors of the PD-1/PD-L pathway. In some embodiments, two or more inhibitors of the PD-1/PD-L pathway may be administered, and in some embodiments, the two or more inhibitors of the PD-1/PD-L pathway may be administered in combination, as a cocktail. In some embodiments, one or more inhibitors of the PD-1/PD-L pathway include one or more antibodies against PD-1, PD-L1, and/or PD-L2. In some embodiments, the method further includes the administration of one or more inhibitors of the CTLA4 pathway.

The present invention includes a method of treating a subject with an infection, the method including administering to the subject an inhibitor of indoleamine-2,3-dioxygenase (IDO) and one or more inhibitors of the PD-1/PD-L pathway. In some embodiments, two or more inhibitors of the PD-1/PD-L pathway may be administered, and in some embodiments, the two or more inhibitors of the PD-1/PD-L pathway may be administered in combination, as a cocktail. In some embodiments, one or more inhibitors of the PD-1/PD-L pathway include one or more antibodies against PD-1, PD-L1, and/or PD-L2. In some embodiments, the method further includes the administration of one or more inhibitors of the CTLA4 pathway.

The present invention includes a method of enhancing an immune response including administering an inhibitor of indoleamine-2,3-dioxygenase (IDO) and one or more inhibitors of the CTLA4 pathway. In some embodiments, the inhibitors of the CTLA4 pathway may include one or more antibodies against CTLA4. In some embodiments, the method further includes administering one or more inhibitors of the PD-1/PD-L pathway.

The present invention includes a method to enhance an immune response to an antigen in a subject, the method including administering to the subject an effective amount of such an antigen in combination with an inhibitor of IDO and one or more inhibitors of the CTLA4 pathway. In some embodiments, the inhibitors of the CTLA4 pathway may include one or more antibodies against CTLA4. In some embodiments, the method further includes administering one or more inhibitors of the PD-1/PD-L pathway.

The present invention includes a method of reducing immune suppression mediated by regulatory T cells (Tregs) in a subject, the method including administering to the subject an inhibitor of indoleamine-2,3-dioxygenase (IDO) and one or more inhibitors of the CTLA4 pathway. In some embodiments, the inhibitors of the CTLA4 pathway may include one or more antibodies against CTLA4. In some embodiments, the method further includes administering one or more inhibitors of the PD-1/PD-L pathway.

The present invention includes a method of enhancing a T cell mediated immune response, the method including administering to the subject an inhibitor of indoleamine-2,3-dioxygenase (IDO) and one or more inhibitors of the CTLA4 pathway. In some embodiments, the inhibitors of the CTLA4 pathway may include one or more antibodies against CTLA4. In some embodiments, the method further includes administering one or more inhibitors of the PD-1/PD-L pathway.

The present invention includes a method of treating cancer in a subject, the method including administering to the subject an inhibitor of indoleamine-2,3-dioxygenase (IDO) and one or more inhibitors of the CTLA4 pathway. In some embodiments, the inhibitors of the CTLA4 pathway may include one or more antibodies against CTLA4. In some embodiments, the method further includes administering one or more inhibitors of the PD-1/PD-L pathway.

The present invention includes a method of treating a subject with an infection, the method including administering to the subject an inhibitor of indoleamine-2,3-dioxygenase (IDO) and one or more inhibitors of the CTLA4 pathway. In some embodiments, the inhibitors of the CTLA4 pathway may include one or more antibodies against CTLA4. In some embodiments, the method further includes administering one or more inhibitors of the PD-1/PD-L pathway.

In some embodiments of the methods of the present invention, the method may further include the administration of an additional therapeutic agent. In some embodiments, the additional therapeutic agent is a cytotoxic chemotherapeutic agent.

In some embodiments of the methods of the present invention, the antigen may be a tumor antigen. In some embodiments, the tumor antigen is delivered as a vaccine, a recombinant viral vector, or autologous or allogeneic tumor cells or cell line.

In some embodiments of the methods of the present invention, the subject may be a patient with cancer.

In some embodiments of the methods of the present invention, the inhibitor of IDO may be 1-methyl-tryptophan (1-MT). In some embodiments, 1-MT may be selected from the group consisting of an isolated D isomer of 1-MT, an isolated L isomer of 1-MT, and a racemic mixture of 1-MT.

The term “and/or” means one or all of the listed elements or a combination of any two or more of the listed elements.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

The terms “comprises” and variations thereof do not have a limiting meaning where these terms appear in the description and claims.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIGS. 1A-1D present Treg activation by DCs from TDLNs. FIG. 1A shows immunohistochemical staining for IDO protein of TDLNs and contralateral LN from mice with B16F10 and B78H1-GMCSF tumors. Overall magnification, ×200. In FIG. 1B, TDLNs and contralateral LNs were stained for CD4 versus intracellular Foxp3. Quadrant percentages are shown. In FIG. 1C, Tregs (CD4⁺CD25⁺) from TDLNs or contralateral LNs were sorted and added to readout assays comprising 1×10⁵ A1 T cells plus CBA DCs plus H-Y peptide. Proliferation ([³H]thymidine incorporation) is shown, with the ratio of Tregs to A1 cells shown below the axis (bars show SD of replicate wells). The lower graph shows data from eight independent experiments using the tumor types shown. In FIG. 1D, CD11c⁺ DCs were harvested from TDLNs, pulsed with OVA peptide, and injected subcutaneously into recipient mice pre-loaded with OT-I T cells. One group of mice received implantable sustained-release 1MT pellets at 5 mg/day (“IDO blocked”), while the other received vehicle control pellets (“IDO active”). After 4 days, the LNs draining the site of DC injection were harvested and the Tregs sorted and tested in vitro for spontaneous suppressor activity in readout assays (A1 T cells plus CBA DCs).

FIGS. 2A-2E show activation of Tregs by IDO in vitro. In FIG. 2A, resting Tregs were cocultured with TDLN pDCs plus OT-1 T cells plus feeder cells. As controls, Tregs were pre-activated in identical cultures with 1MT added to block IDO activity. Graph shows the mean of 5-8 pooled experiments, using pDCs from B78H1-GMCSF and B16-OVA tumors; bars show SD. In FIG. 2B, Tregs were activated as above, or in identical cultures containing 1MT to block IDO plus anti-CD3 mAb plus IL-2 to activate the Tregs. In FIG. 2C,

Tregs were activated in cocultures as above, with the APCs being either TDLN pDCs; non-pDC fraction from the same TDLN (CD11c⁺B220^{NEG}); pDCs from mice without tumors; or TDLN pDCs from IDO-KO mice. Bars show SD of replicate wells. In FIG. 2D, Tregs were activated with TDLNs pDCs with or without 1MT. In FIG. 2E, IDO-activated Tregs were sorted and added to readout assays containing A1 T cells plus either CBA DCs or CBA B cells.

FIGS. 3A-3D show suppression by IDO-activated Tregs requires the PD-1/PD-L pathway. In FIG. 3A, Tregs were activated with IDO⁺ pDCs, then 1×10⁴ sorted Tregs were added to readout assays and DCs were stained to test for the presence or absence of the DC-associated molecules PD-L1 and PD-L2. In FIG. 3B, IDO activated Tregs (5000/well) were added to readout assays (A1 T cells plus either wild-type CBA DCs or IDO-KO DCs on the CBA background). Readout assays received either no additive, 1MT, or a cocktail of blocking antibodies against PD-1, PD-L1 and PD-L2 (50 ug/ml each). Control Tregs received 1MT during the pre-activation step. In FIG. 3C, Tregs were activated with IDO⁺ pDCs, or in identical cultures containing 1MT to block IDO and anti-CD3+IL-2 to activate the Tregs. After sorting, Tregs were added to readout assays (A1 T cells plus CBA DCs), with or without PD-1/PD-L blocking antibodies as shown. In FIG. 3D, IDO-activated Tregs (1×10⁴/well) and anti-CD3/IL-2-activated Tregs (2×10⁴/well) were prepared, and added to readout assays with or without recombinant IL-2, anti-IL-10+anti-TGFβ blocking antibodies (100 ug/ml each), or PD-1/PD-L blocking antibodies. Bars show SD for replicate wells.

FIGS. 4A-4D show IDO-induced activation requires GCN2-kinase in Tregs. In FIG. 4A, activation cultures were set up with Tregs, TDLN pDCs, OT-I cells and feeder cells, with or without 1MT. After 2 days, intracellular staining was performed for CHOP expression in Tregs (CD4+ cells). The percentages show the fraction of Tregs that were CHOP⁺. FIG. 4B shows Tregs derived from wild-type mice versus GCN2-KO mice (each assay with OVA, without 1MT). In FIG. 4C, Tregs from GCN2-KO mice or wild-type controls were activated with IDO⁺ pDCs, resorted, and 5000 Treg added to readout assays (A1 T cells plus CBA DCs), with and without PD-1/PD-L blocking antibodies. In FIG. 4D, Tregs from wild-type mice were activated with IDO⁺ pDCs with or without 10× tryptophan (250 μM), resorted, and tested in readout assays with and without added 10× tryptophan (250 uM). Bars show SD for replicate wells.

FIGS. 5A-5C show MHC-dependent and independent steps in IDO-induced Treg activation. In FIG. 5A, B6 Tregs were activated with IDO⁺ pDCs, with or without anti-CTLA4 blocking mAb (10 ug/ml) during the pre-activation step. Bars show SD for replicate wells. FIG. 5B shows CHOP induction in Tregs is MHC-restricted. The left-hand plot shows assays using Tregs that were MHC matched to the IDO⁺ pDCs (B6 background); the second plot shows assays with MHC mismatched (CBA) Tregs. The final plot shows cultures with MHC-matched B6 Tregs but with 100 ug/ml blocking antibody to Ia^{b}. Controls without blocking antibody, or with irrelevant antibody, were similar to the first plot. In FIG. 5C (left-hand graph), activation cocultures were set up using MHC mismatched (CBA) Tregs. In FIG. 5C (right-hand graphs), identical assays, except that CBA Tregs were mixed with Thy1.1 congenic B6 Tregs (10,000 each) during the activation cocultures, then each Treg population was resorted and tested separately. Bars show SD for replicate wells in one of three similar experiments, using TDLN pDCs from B78H1-GMCSF and B16-OVA tumors.

FIGS. 6A and 6B show direct activation of mature Tregs is more potent than de novo differentiation of new Tregs. In FIG. 6A, activation cocultures were set up using Thy1.1-congenic B6 Tregs. To these were added CD4+CD25NEG (naive, non-regulatory) T cells from A1 mice plus CBA spleen DCs. Parallel groups received either no H-Y antigen for the A1 cells, H-Y, or H-Y+1MT. All cultures received OVA peptide for the OT-I cells. After two days, co-cultures were stained for CD4 versus Foxp3 versus Thy1.1. The inset dot-plots show similar cultures in which the A1 and OT-I cells were labeled with CFSE prior to addition and analyzed for cell division at the end of the assay. CFSE histograms for the A1 cells (CD4⁺ CFSE⁺) are superimposed. In FIG. 6B, assays were set up as in the previous panel, using Thy1.1 congenic Tregs plus nonregulatory CD4+CD25NEG cells from wild-type B6 mice, activated with anti-CD3 mAb. Inset dot dotplots document upregulation of Foxp3 in this model, using CD4⁺CD25^{NEG} cells pre-labeled with CFSE. After two days the Treg and non-Treg populations were sorted separately based on

Thy1.1 expression, and tested in readout assays (A1 T cells+CBA DCs). Bars show SD.

FIGS. 7A-7D show IDO-activated Tregs in TDLNs. In FIG. 7A, tumors were grown in wild-type or IDO-KO hosts. Tregs from day seven TDLNs were sorted and added to readout assays (A1 T cells+CBA DCs), with and without PD-1/PD-L blocking antibodies. Means of four pooled experiments with B78H1-GMCSF, four experiments with B16-OVA, and three experiments with IDO-KO hosts (two with B78H1-GMCSF and one with B16-OVA). In FIG. 7B, wild-type mice were treated throughout tumor growth with vehicle control (“IDO active”) or sustained-release 1MT (“IDO blocked”). Tregs from day seven tumors were tested in readout assays as above, with added isotype, PD-1/PD-L blocking antibodies, or a combination of anti-PD-1/PD-L plus IL-2 plus anti-IL-10/TGF-β antibodies. In FIG. 7C (upper panels), CFSE-labeled OT-I cells were injected into mice with B16-OVA tumors (day 7-8), with and without oral 1MT administration after transfer. After four days, TDLNs and contralateral LNs (CLN) were stained for the 1B11 activation marker. Percentages show the CFSE+ OT-I cells in total LN cells. Overlay histogram shows 1B11 on OT-I cells in TDLNs. In FIG. 7C (lower panels), similar experiments, using OT-IG CN2-KO cells transferred into WT or GCN2-KO hosts bearing B16-OVA tumors. In FIG. 7D, mice bearing B78H1-GMCSF tumors were treated on day 11 with vehicle (control), cyclophosphamide (CY, 150 mg/kg), or CY+1MT pellets. Seven days later, cells from TDLNs were harvested and added to readout assays (allospecific BM3 T cells plus B6 splenocytes). One control received 1MT added to the readout assay.

FIG. 8 presents a model of IDO-induced Treg activation.

FIGS. 9A and 9B demonstrate IDO expression by the CD19⁺ cells in the pDC fraction of TDLNs. FIG. 9A is an immunohistochemical staining of cytocentrifuge preparations of sorted CD19⁺ pDCs (CD11c⁺B220⁺CD19⁺) from TDLNs of B78H1-GMCSF tumors. FIG. 9B presents FACS plots of gated B220⁺ cells from TDLNs, showing the CD11c⁺ CD19⁺ subset (the CD19⁺ pDCs sorted at left).

FIG. 10 shows IDO-activated Tregs can suppress CD8⁺ T cells. Tregs were activated for two days in coculture with TDLN pDCs plus OT-I cells plus OVA peptide. Activated Tregs were harvested, resorted, and added to readout assays comprising CD8⁺ OT-I T cells plus CD11c splenic DCs from B6 mice plus OVA peptide.

FIG. 11 shows Tregs mediate suppression of bystander A1 cells in mixed cocultures. □ represents (−) 1MT and (+) anti-CD3; A represents (−) 1MT and (−) anti-CD3; ▪ represents (+) 1MT and (+) anti-CD3; and ▴ represents (+) 1MT and (−) anti-CD3.

FIG. 12 shows suppressed A1 cells upregulate activation markers but do not divide. In FIG. 12A, mixed cocultures were established, comprising Treg activation cultures (IDO⁺ pDCs, OT-I cells, Tregs, and feeder layer) plus the direct addition of CFSE-labeled CD4⁺ sorted A1 T cells plus CBA DCs plus HY peptide. After 2-3 days co-cultures were harvested and stained for CD25 vs. CFSE. Percentages show the fraction of A1 cells that were. CD25+. In FIG. 12B, IDO-activated Tregs were sorted and added to readout assays of A1 cells plus CBA DCs plus HY peptide. After three days, cells were harvested and stained for CD4 versus annexin V-PE.

FIG. 13 shows Tregs increase IDO enzymatic activity in a CTLA4-dependent fashion. TDLN pDCs (1×10⁴) and OT1 T cells (1×10⁵) were cultured for three days, with or without 1×10⁴ Tregs. Replicate wells received 10 ug/ml anti-CTLA4-blocking antibody (clone 9H10), and/or 1MT, as shown. After three days, the culture supernatants were analyzed by HPLC for the concentration of kynurenine. The arrows show that the addition of Tregs to the culture increased the production of kynurenine above the basal level produced by the IDO⁺ pDCs and OT-I alone; and that this Treg-induced increase was blocked by anti-CTLA4 mAb. The basal level of IDO, which was fully sufficient to inhibit the proliferation of the OT-I cells, was not blocked by anti-CTLA4 mAb (second bar).

FIG. 14 shows IDO-induced Treg activation cannot be created when the medium contains insufficient tryptophan. Cultures were set up containing TDLN pDCs+Tregs+OT-I+feeder cells, with normal or low concentrations of tryptophan in the medium.

FIGS. 15A and 15B show that IDO-activated Tregs create bystander suppression by the activating the PD-1/PD-ligand system in bystander cells. In

FIG. 15A, IDO-activated Tregs potently suppressed the readout assays. FIG. 15B demonstrates the effect on Treg-mediated suppression of either 1MT added to the readout assay, or a cocktail of antibodies against the T cell inhibitory receptor PD 1 and its ligands PD L1 and PD L2 (50 μg/ml each).

FIG. 16 shows IDO-activated Tregs upregulated PDL1 and PDL2 expression on the DCs in the readout assay.

FIGS. 17A-17B show blocking PD1 and both PDL1 and PDL2 prevents suppression by activated Tregs. In FIG. 17A, wild-type cells (WT) DCs were used. In FIG. 17B, PDL1/L2 double knock out (PDL1/L2-DKO) DCs were used.

FIGS. 18A-18C demonstrate Tregs trigger super-induction of IDO in pDCs. In FIG. 18A bystander assays were performed in transwell chambers with the cells distributed as shown. Bar graphs show [³H]thymidine incorporation, measured separately in each chamber, with or without 1MT added to both chambers. In FIG. 18B supernatants from bystander assays, with or without Tregs, were analyzed by HPLC for kynurenine. Cultures for HPLC analysis contained 5× the usual number of pDCs. In FIG. 18C the generation of soluble suppressive factor is prevented in low tryptophan. Suppressor assays (pDCs+Tregs+OT-I+feeder cells) were set up in medium with various concentrations of tryptophan. After eighteen hours, the supernatant was harvested and added at a 1:1 dilution to readout assays (A1 cells+CBA DCs).

FIG. 19 demonstrates that antigen presentation to OT-I cells is required to trigger functional IDO enzyme activity. IDO activity was measured as tryptophan depletion and kynurenine production in culture supernatants. Assays were performed with and without the cognate OVA peptide (SIINFEKL) (SEQ ID NO:1) to activate the OT-I cells. The HPLC traces show the kynurenine and tryptophan peaks for groups with and without OVA. The concentration (in μM) of tryptophan and kynurenine in the medium is shown above each peak, interpolated from a standard curve.

### DETAILED DESCRIPTION OF ILLUSTRATIVE EMBODIMENTS OF THE INVENTION

The present invention demonstrates that a small population of plasmacytoid dendritic cells (pDCs) in tumor-draining lymph nodes (TDLN) can express the immunoregulatory enzyme indoleamine 2,3-dioxygenase (IDO). These IDO⁺ pDCs directly activate resting CD4⁺CD25⁺Foxp3⁺ Tregs for potent suppressor activity. In vivo, Tregs isolated from TDLNs were constitutively pre-activated, and suppressed antigen-specific T cells immediately ex vivo. In vitro, IDO⁺ pDCs from TDLNs rapidly activated resting Tregs from non-tumor-bearing hosts, without the need for mitogen or exogenous anti-CD3 crosslinking. This Treg activation by IDO⁺ pDCs was MHC-restricted, required intact general control nonderepressing-2 (GCN2) kinase in the Tregs, and was prevented by blockade of CTLA4. Tregs activated by IDO markedly upregulated PD-L1 and PD-L2 expression on target DCs, and the ability of Tregs to suppress target T cell proliferation was abrogated by antibodies against the PD-1/PD-ligand pathway. In contrast, Tregs activated by anti-CD3 crosslinking did not cause upregulation of PD-ligands, and suppression by these cells was unaffected by blocking the PD-1/PD-ligand pathway. Tregs isolated from tumor-draining LNs in vivo showed potent PD-1/PD-ligand mediated suppression, which was selectively lost when tumors were grown in IDO-deficient hosts. Thus, IDO⁺ pDCs create a profoundly suppressive microenvironment within TDLNs via constitutive activation of Tregs.

The present invention demonstrates, for the first time, a mechanistic link between the immunoregulatory enzyme indoleamine 2,3-dioxygenase (IDO), functional activation of regulatory T cells (Tregs), and the programmed cell death 1/PD-ligand (PD-1/PD-L) pathway, a mechanistic link at the level of Treg activation in an antigen stimulated lymph node. The present invention demonstrates that IDO⁺ DCs condition or activate Tregs in such a way that they can further induce expression of PD-L1/PD-L2 in lymph node DCs which leads to subsequent IDO-independent suppression of T cell proliferation mediated by the PD-1/PD-L1/PD-L2 pathway. This linked pathway constitutes a major contributor to the intensely immunosuppressive milieu present in tumor draining lymph nodes (TDLNs). Since this suppressive milieu drives T cell anergy and unresponsiveness to tumor antigens presented in the TDLNs (Munn et al., 2006, Immunol. Rev; 213:146-158), identification of the molecular mechanisms contributing to immunosuppression and T cell anergy represent important advances for the treatment of cancer, viral infections, autoimmunity, transplants, vaccinations, allergic reactions, and chronic infections.

Further, the present invention demonstrates that a small population of plasmacytoid dendritic cells (pDCS) expresses IDO and that these IDO⁺ pDCS directly activate resting Tregs for potent immunosuppresor activity. The present invention demonstrates IDO⁺ pDCS create an immunosuppressive microenvironment within a draining lymph node, such as a tumor draining lymph node, via the constitutive activation of Tregs. The present invention demonstrates that Treg activation can be prevented by IDO inhibitors and that the ability of activated Tregs to suppress T cell proliferation can be abrogated by IDO inhibitors and inhibitors of the PD-1/PD-L pathway. Furthermore, the present invention demonstrates that anti-CTLA4 can be used to reduce IDO activity induced by the presence of CTLA4⁺ Tregs.

The discovery of the link between the IDO pathway activating Tregs which further induce upregulation of PD-L1/L2 immunosuppressive molecules in dendritic cells indicates that the immunostimulatory therapeutic potency of IDO inhibitors and anti-PD1/anti-PDL1/anti-PDL2 can be increased if these two therapeutic alternatives are combined. Furthermore, the present invention demonstrates that IDO activity can be induced by CTLA4⁺ Tregs and that IDO activity can be partially reversed by anti-CTLA4. To obtain full suppression of IDO activity in an IDO⁺ DC, a combination of IDO inhibitors with CTLA4 blockade presents a new therapeutic approach.

Thus, with the present invention, IDO inhibitor can be administered along with inhibitors of the PD-1/PD-L pathway and/or inhibitors of the CTLA4 pathway to modulate immune responses controlled by the activation of Tregs. For example, IDO inhibitors can be administered along with one or more inhibitors of the PD-1/PD-L pathway and/or one or more inhibitors of the CTLA4 pathway to alter an immune response, both in vitro and in vivo. IDO inhibitors can be administered along with one or more inhibitors of the PD-1/PD-L pathway and/or one or more inhibitors of the CTLA4 pathway in methods of enhancing an immune response in a subject, enhancing the immune response to an antigen in a subject, suppressing the induction of Tregs in a subject, suppressing the generation or activation of Tregs in a subject, reducing immune suppression mediated by Tregs in a subject, enhancing a T cell mediated immune response, treating cancer, augmenting the rejection of a tumor, and/or reducing tumor size in a subject, and treating a subject with an infection. The administration of one or more IDO inhibitors along with one or more inhibitors of the PD-1/PD-L pathway and/or one or more inhibitors of the CTLA4 pathway may demonstrate a synergistic effect on an immune response.

IDO activates Tregs for a novel mechanism of suppression (see PCT/US2007/000404; “Indoleamine 2,3-Dioxygenase Pathways in the Generation of Regulatory T Cells,”). IDO-induced activation differs from conventional Treg activation in that it is rapid, extremely potent, and independent of mitogen or other external activating stimuli. The present invention shows that a mechanism of suppression by IDO-activated Tregs is the induction of the suppressive PD-1/PD-ligand pathway in bystander cells. This is a new mechanism of Treg activity, not previously described. The findings of the present invention mechanistically link IDO⁺ pDCs, activated Tregs, and the potent PD-1/PD-ligand system, which has recently been recognized as a major mechanism of anergy and clonal exhaustion in effector T cells. The present invention shows that IDO-induced Treg activation is present at high levels in Tregs isolated directly from tumor draining LNs, and is absent in IDO-knockout mice and mice treated with IDO-inhibitor drug, confirming the in vivo biologic relevance of the pathway for tumor immunology.

Although most auto-reactive T lymphocytes are regulated and eliminated during thymic development, healthy individuals continue to carry self-reactive cells. T regulatory cells (Tregs) are an immunoregulatory cell type used to control autoimmunity in the periphery. Tregs are CD4 positive. The constitutive expression of CD25 is considered to be a characteristic feature of human Tregs. Thus, Tregs are often CD4⁺CD25⁺ T cells. Tregs are potent suppressors of T cell mediated immunity in a range of inflammatory conditions, including infectious disease, autoimmunity, pregnancy and tumors (Sakaguchi, 2005, Nat Immunol; 6:345-352). Mice lacking Tregs die rapidly of uncontrolled autoimmune disorders (Khattri et al., 2003, Nat Immunol; 4:337-342). In vivo, a small percentage of Tregs can control large numbers of activated effector T cells. Although freshly isolated Tregs exhibit minimal constitutive suppressor functions, ligating the T cell antigen receptor (TCR) in vitro (Thornton et al. 2004, Eur J Immunol; 34:366-376), or pre-immunizing mice with high-dose self-antigen in vivo stimulates Treg suppressor functions (Nishikawa et al., 2005, J Exp Med; 201:681-686). This requirement for TCR signaling to enhance Treg suppressor functions is paradoxical because most Tregs are thought to recognize constitutively expressed self-antigens (Nishikawa et al., 2005, J Exp Med; 201:681-686; Hsieh et al., 2004, Immunity; 21:267-277; Fisson et al., J Exp Med; 198:737-746; Kronenberg et al., 2005, Nature; 435:598-604). The present invention shows that increased IDO activity increases suppressive functions mediated by Tregs and that the inhibition of IDO activity abrogates these suppressive functions.

Tregs of the present invention may express CD4 (CD4⁺) and/or CD25 (CD25⁺). Tregs of the present invention may also be positive for the transcriptional repression factor forkkhead box P3 (FoxP3). Tregs of the present invention may express a high affinity IL-2 receptor. Tregs of the present invention may be CD8⁺ Tregs. Tregs have been studied for more than thirty years and are further reviewed in, for example, Beyer and Schultze, 2006, Blood; 108:804-11; Elkord, 2006, Inflamm Allergy Drug Targets; 5:211-7; Ghiringhelli et al., 2006, Immunol Rev; 214:229-38; Jiang et al., 2006, Hum Immunol; 67:765-76; Kabelitz et al., 2006, Crit Rev Immunol; 26:291-306; Le and Chao, 2007, Bone Marrow Transplant; 39:1-9; Sakaguchi et al., 2006, Immunol Rev; 212:8-27; Shevach et al., 2006, Immunol Rev; 212:60-73; Stein-Streilein and Taylor, 2006, J Leukoc Biol, 81:593-8; and Wing and Sakaguchi, 2006, Curr Opin Allergy Clin Immunol; 6:482-8.

A recently discovered molecular mechanism contributing to peripheral immune tolerance is the immunoregulatory enzyme indoleamine 2,3-dioxygenase (IDO). Cells expressing the tryptophan catabolizing enzyme IDO are capable of inhibiting T cell proliferation in vitro and reducing T cell immune responses in vivo (U.S. Pat. Nos. 6,451,840 and 6,482,416; PCT/US2007/000404; Munn et al., 1998, Science; 281:1191; Munn et al., 1999, J Exp Med; 189:1363; Hwu et al., 2000, J Immunol; 164:3596; Mellor et al., 2002, J Immunol; 168:3771; Grohmann et al., 2001, J Immunol; 167:708; Grohmann et al., 2001, J Immunol; 166:277; and Alexander et al., 2002, Diabetes; 51:356).

IDO degrades the essential amino acid tryptophan (for reviews see Taylor et al., 1991, FASEB J; 5:2516-2522; Lee et al., 2003, Laboratory Investigation; 83:1457-1466; and Grohmann et al., 2003, Trends Immunol; 24:242-248). Expression of IDO by human monocyte-derived macrophages (Munn et al., 1999, J Exp Med; 189:1363-1372), human dendritic cells (Munn et al., 2002, Science; 297:1867-1870 and Hwu et al., 2000, J Immunol; 164:3596-3599), and mouse dendritic cells (Mellor et al., 2003, J Immunol; 171:1652-1655) allows these different antigen-presenting cells (APCs) to inhibit T cell proliferation in vitro. In vivo, IDO participates in maintaining maternal tolerance toward the antigenically foreign fetus during pregnancy (Munn et al., 1998, Science; 281:1191-1193).

IDO has also been implicated in maintaining tolerance to self antigens (Grohmann et al., 2003, J Exp Med; 198:153-160), in suppressing T cell responses to MHC-mismatched organ transplants (Miki et al., 2001, Transplantation Proceedings; 33:129-130; Swanson, et al., 2004, Am J Respir Cell Mol Biol; 30:311-8; Beutelspacher et al., 2006, Am J Transplant; 6:1320-30) and in the tolerance-inducing activity of recombinant CTLA4-Ig (Grohmann et al., 2002, Nature Immunol; 3:985-1109; Mellor et al., 2003, J Immunol; 171:1652-1655) and the T cell regulatory functions of interferons (Grohmann et al., 2001, J Immunol; 167:708-14; and Baban et al., 2005, Int Immunol; 17:909-919). In these four systems, the immunosuppressive effect of IDO can be blocked by the in vivo administration of an IDO inhibitor, such as 1-methyl-tryptophan (also referred to herein as 1-MT or 1MT).

The transfection of IDO into mouse tumor cell lines confers the ability to suppress T cell responses both in vitro and in vivo (Mellor et al., 2002, J Immunol; 168:3771-3776). In a Lewis Lung carcinoma model, administration of 1-MT significantly delayed tumor outgrowth (Friberg et al., 2002, Int J Cancer; 101:151-155). The mouse mastocytoma tumor cell line P815 forms lethal tumors in naive hosts, but is normally rejected by pre-immunized hosts. However, transfection of P815 with IDO prevents its rejection by pre-immunized hosts (Uyttenhove et al., 2003, Nature Medicine; 9:1269-1274). Inhibition of tumor growth was entirely dependent on the presence of an intact immune system and was substantially reversed, that is, tumor growth inhibited, by the concomitant administration of 1-MT.

The selective recruitment of IDO⁺ APCs in the tumor-draining (sentinel) lymph nodes of patients with melanoma (Munn et al., 2002, Science; 297:1867-1870 and Lee et al., 2003, Laboratory Investigation; 83:1457-1466) indicates that tumors take advantage of the immunosuppressive effect of IDO by recruiting a population of IDO-expressing host APCs to present tumor antigens. Similar changes have been seen in breast carcinoma and other tumor-associated lymph nodes. In mouse tumor models the IDO-expressing APCs in tumor-draining lymph nodes are phenotypically similar to a subset of dendritic cells recently shown to mediate profound IDO-dependent immunosuppressive in vivo (Mellor et al., 2003, J Immunol; 171:1652-1655; and Baban et al., 2005, Int Immunol; 17:909-919). IDO-expressing APCs in tumor-draining lymph nodes thus constitute a potent tolerogenic mechanism.

The IDO enzyme is well characterized (see, for example, Taylor et al., 1991, FASEB J; 5:2516-2522; Lee et al., 2003, Laboratory Investigation; 83:1457-1466; and Grohmann et al., 2003, Trends Immunol; 24:242-248) and compounds that serve as substrates or inhibitors of the IDO enzyme are known. For example, Southan (Southan et al., 1996, Med. Chem Res; 343-352) utilized an in vitro assay system to identify tryptophan analogues that serve as either substrates or inhibitors of human IDO. Methods for detecting the expression of IDO in cells are well known and include, but are not limited to, any of those described herein and those described, for example in U.S. Pat. Nos. 6,395,876, 6,451,840, and 6,482,416, U.S. Patent Application Nos. 20030194803, 20040234623, 20050186289, and 20060292618, PCT/US2006/040796, and PCT/US2007/000404.

The present invention includes methods of affecting an immune response by administering an inhibitor of IDO along with one or more inhibitors of the PD-1/PD-L pathway. An inhibitor of IDO may be administered coincident with the administration of one or more additional inhibitors. An inhibitor of IDO may be administered before or after the administration of one or more additional inhibitors. An inhibitor of IDO and one or more additional inhibitors may be administered separately or as a part of a mixture or cocktail. Affecting an immune response, includes, but is not limited to, enhancing an immune response, suppressing the generation of Tregs, reducing the immune suppression mediated by Tregs, reducing the induction of antigen-specific Tregs, enhancing an immune response to an antigen, and/or enhancing the immunostimulatory capacity of DCs to tumor antigens. In some aspects of the present invention, IDO inhibitors along with one or more inhibitors of the PD-1/PD-L pathway may demonstrate synergistic activity. In some aspects of the present invention, the administration of one or more IDO inhibitors may allow for the effectiveness of a lower dosage of one or more inhibitors of the PD-1/PD-L pathway when compared to the administration of one or more inhibitors of the PD-1/PD-L pathway alone, providing relief from the toxicity observed with the administration of higher doses of inhibitors of the PD-1/PD-L pathway.

The present invention includes methods of affecting an immune response by administering an inhibitor of IDO along with one or more inhibitors of the CTLA4 pathway. An inhibitor of IDO may be administered co-incident with the administration of one or more additional inhibitors. An inhibitor of IDO may be administered before of after the administration of one or more additional inhibitors. An inhibitor of IDO and one or more additional inhibitors may be administered separately or as a part of a mixture or cocktail. Affecting an immune response, includes, but is not limited to, enhancing an immune response, suppressing the generation of Tregs, reducing the immune suppression mediated by Tregs, reducing the induction of antigen-specific Tregs, enhancing an immune response to an antigen, and/or enhancing the immunostimulatory capacity of DCs to tumor antigens. In some aspects of the present invention, IDO inhibitors along with one or more inhibitors of the CTLA4 pathway may demonstrate synergistic activity. In some aspects of the present invention, the administration of one or more IDO inhibitors may allow for the effectiveness of a lower dosage of one or more inhibitors of the CTLA4 pathway when compared to the administration of one or more inhibitors of the CTLA4 pathway alone, providing relief from the toxicity observed with the administration of higher doses of inhibitors of the CTLA4 pathway.

The present invention also includes methods of affecting an immune response by administering an inhibitor of IDO along with both one or more inhibitors of the PD-1/PD-L pathway and one or more inhibitors of the CTLA4 pathway. An inhibitor of IDO may be administered co-incident with the administration of one or more additional inhibitors. An inhibitor of IDO may be administered before of after the administration of one or more additional inhibitors. An inhibitor of IDO and one or more additional inhibitors may be administered separately or as a part of a mixture or cocktail. Affecting an immune response, includes, but is not limited to, enhancing an immune response, suppressing the generation of Tregs, reducing the immune suppression mediated by Tregs, reducing the induction of antigen-specific Tregs, enhancing an immune response to an antigen, and/or enhancing the immunostimulatory capacity of DCs to tumor antigens. In some aspects of the present invention, IDO inhibitors along with one or more inhibitors of the PD-1/PD-L pathway and or one or more inhibitors of the CTLA4 pathway may demonstrate synergistic activity. In some aspects of the present invention, the administration of one or more IDO inhibitors may allow for the effectiveness of a lower dosage of one or more inhibitors of the PD-1/PD-L pathway and/or one or more inhibitors of the CTLA4 pathway when compared to the administration of inhibitors of the PD-1/PD-L and CTLA4 pathways alone.

An IDO inhibitor is an agent capable of inhibiting the enzymatic activity of indoleamine 2,3-dioxygenase (IDO). An IDO inhibitor may be a competitive, noncompetitive, or irreversible IDO inhibitor. A competitive IDO inhibitor is a compound that reversibly inhibits IDO enzyme activity at the catalytic site (for example, without limitation, 1-methyl-tryptophan), a noncompetitive IDO inhibitor is a compound that reversibly inhibits IDO enzyme activity at a non-catalytic site (for example, without limitation, norharman), and an irreversible IDO inhibitor is a compound that irreversibly destroys IDO enzyme activity by forming a covalent bond with the enzyme (for example, without limitation, cyclopropyl/aziridinyl tryptophan derivatives).

A wide variety of IDO inhibitors are well known to the skilled artisan, and include, but are not limited to antibodies, peptides, nucleic acid molecules (including, for example, an antisense molecule, a PNA, or an RNAi), peptidomimetics, and small molecules. In a preferred embodiment, an IDO inhibitor is a small molecule inhibitor of IDO.

Small molecule inhibitors of IDO include, but are not limited to, any of a variety of commercially available IDO inhibitors, such as, but not limited to, 1-methyl-DL-tryptophan (also referred to herein as “1 MT,” “1-MT,” or “1MT”) (Sigma-Aldrich; St. Louis, Mo.), β-(3-benzofuranyl)-DL-alanine (Sigma-Aldrich), beta-(3-benzo(b)thienyl)-DL-alanine (Sigma-Aldrich), 6-nitro-L-tryptophan (Sigma-Aldrich), indole 3-carbinol (LKT Laboratories; St. Paul, Minn.), 3,3′-diindolylmethane (LKT Laboratories), epigallocatechin gallate (LKT Laboratories), 5-Br-4-Cl-indoxyl 1,3-diacetate (Sigma-Aldrich), 9-vinylcarbazole (Sigma-Aldrich), acemetacin (Sigma-Aldrich), 5-bromo-DL-tryptophan (Sigma-Aldrich), and 5-bromoindoxyl diacetate (Sigma-Aldrich). Small molecule inhibitors of IDO include, for example, any of the many competitive and noncompetitive inhibitors of IDO discussed in Muller et al. (Muller et al. 2005, Expert Opin Thr Targets; 9:831-849).

IDO inhibitors of the instant invention may include, but are not limited to, any of a variety of the small molecule inhibitors of IDO described in US Patent Applications Nos. 20060258719, 20070203140 (including, but not limited to various N-hydroxyguanidines compounds), 20070185165 (including, but not limited to, various N-hydroxyamidinoheterocycles compounds), 20070173524 (including, but not limited to, various brassilexin and brassinin derivatives), and 20070105907 (including, but not limited to, various brassilexin and brassinin derivatives), WO 2004/094409, PCT/US2004/005154, WO/2006/005185 (naphtoquinones derivatives), PCT/CA2005/001087, Gaspari et al., 2006, J Med Chem; 49:684-92 (brassinin derivatives), Muller et al., 2005, Nat. Med; 11:312-319, Peterson et al., 1993, Med Chem Res; 3:473-482 (substituted beta-carbolines), Sono et al., 1989, Biochemistry; 28:5392-9, Sono et al., 1996, Chem Rev; 96:2841, and Vottero et al., 2006, Biotechnol J; 1:282-288.

IDO inhibitors of the instant invention may include, for example, any of compounds taught in PCT/US2007/000404, “Indoleamine 2,3-Dioxygenase Pathways in the Generation of Regulatory T Cells,” including, but not limited to, compounds A-YY, and analogs and derivatives thereof.

The present invention also includes pharmaceutically acceptable salts of IDO inhibitors. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts or the quaternary ammonium salts of the parent compound formed, for example, from non-toxic inorganic or organic acids. The pharmaceutically acceptable salts of the present invention can be synthesized from the parent compound which contains a basic or acidic moiety by conventional chemical methods.

In some embodiments of the present invention, an IDO inhibitor may be a racemic mixture of an inhibitor, an isolated D isomer of an inhibitor, or an isolated L isomer of an inhibitor, for example, a racemic mixture of 1-MT, an isolated D isomer of 1-MT, or an isolated L isomer of 1-MT. The purification of D and L isomers can be carried out by any of numerous methods known in the art. In some embodiments, an IDO inhibitor is a D isomer of 1MT, an L isomer of 1MT, or a racemic mixture of 1MT. See, for example, published U.S. Patent Application Nos. 2004/0234623 and 2005/0186289.

Programmed cell death 1 (PD-1, also known as CD279, gene name PDCD1) was isolated in 1992 by subtractive-hybridization technique, as a molecule whose expression is enhanced by apoptotic stimuli (Okazaki and Wang, 2005, Autoimmunity; 38:353-7). PD-1 is a 55 KDa member of the immunoglobulin superfamily. PD-1 is a type I transmembrane protein belonging to the CD28/CTLA-4 family of immunoreceptors, which mediate signals for regulating immune responses. PD-1 is expressed on activated T cells, B cells, myeloid cells and on a subset of thymocytes. Mouse and human

PD-1 share approximately 60% amino acid sequence identity. PD-1 contains the immunoreceptor tyrosine-based inhibitory motif (ITIM) and plays a key role in peripheral tolerance and autoimmune disease. See, for example, Melero et al., 2007, Nat Rev; 7:95-106; Ishida et al., 1992, EMBO J; 11:3887; Shinohara et al., 1994, Genomics; 23:704; U.S. Pat. No. 5,698,520; Honjo, 1992, Science; 258:591; Agata et al., 1996, Int. Immunol; 8:765; Nishimura et al., 1996, Int Immunol; 8:773; and Nishimura, 1998, Int Immunol; 10:1563.

Two members of the B7 family have been identified as ligands for PD-1, PD-L1 (B7-H1, CD274) and PD-L2 (B7-DC, CD273). See, for example, Freeman et al., 2000, J Exp Med; 192:1027; Latchman et al., 2001, Nat Immunol; 2:261-268. Evidence suggests overlapping functions for these two PD-1 ligands. The fact that PD-1 binds to PD-L1/PD-L2 places PD-1 in a family of inhibitory receptors with CTLA4. PD-L1 (B7H1), a member of the B7 family, has a predicted molecular weight of approximately 40 kDa and belongs to the Ig superfamily. PD-L1 is expressed on a majority of leukocytes. Interaction of PD-1 with either PDL1 or PDL2 results in inhibition of T and B cell responses. PD-L2 (B7DC) a recently identified member of the B7 family, has a predicted molecular weight of approximately 25 kDa and it also belongs to the Ig superfamily (see, for example, Latchman et al., 2001, Nat Immunol; 2:1. The nucleotide sequence of a cDNA encoding human PD-L2 is available as Genbank Accession number AF344424 (see Latchman et al., 2001, Nat Immunol; 2:261-268). PD-L2 is primarily expressed by subpopulations of dendritic cells and monocytes/macrophages. Although PD-L2 has structural and sequence similarities to the B7 family, it does not bind CD28/CTLA-4, rather it is a ligand for PD.

Inhibitors of the PD-1/PD-L pathway include, but are not limited to, antibodies, peptides, nucleic acid molecules (including, for example, an antisense molecule, a PNA, or an RNAi), peptidomimetics, small molecules, a soluble PD-1 ligand polypeptide, or a chimeric polypeptide (for example, a chimeric PD-1 ligand/Immunoglobulin molecule). An antibody may be an intact antibody, an antibody binding fragment, or a chimeric antibody. A chimeric antibody may include both human and non-human portions. An antibody may be a polyclonal or a moncoclonal antibody. An antibody may be a derived from a wide variety of species, including, but not limited to mouse and human. An antibody may be a humanized antibody. An antibody may be linked to another functional molecule, for example, another peptide or protein, a toxin, a radioisotype, a cytotoxic agent, cytostatic agent, a polymer, such as, for example, polyethylene glycol, polypropylene glycol or polyoxyalkenes.

One or more inhibitors of the PD-1/PD-L pathway may include a combination of inhibitors of the PD-1/PD-L pathway. For example, one or more inhibitors of PD-1, one or more inhibitors of PD-L1, and/or one or more inhibitors of PD-L2 may be administered. One or more of such inhibitors may be an antibody. For example, to inhibit the PD-1/PD-L pathway a mixture of inhibitors of PD-1, PD-L1, and/or PD-L2 may be used in combination. In some embodiments, one or more inhibitors of PD-1 and one or more inhibitors of PD-L1 may be administered. In some embodiments, one or more inhibitors of PD-1 and one or more inhibitors of PD-L2 may be administered. In some embodiments, one or more inhibitors of PD-1, one or more inhibitors of PD-L1, and one or more inhibitors of PD-2 may be administered. A mixture or cocktail of inhibitors of the PD-1/PD-L pathway may be administered. For example, a cocktail of antibodies to PD-1, PD-L1, and/or PD-L2 may be administered.

Any of a variety of PD-1, PD-L1, and/or PD-L2 antibodies may be used, including, but not limited to, any of those described herein and, for example, those commercially available from, for example, R&D Systems, Invitrogen, BioLegend, eBiosciences, or Acris Antibodies, and those described, for example, in U.S. Patent Application Serial Nos. 2002 0164600; 2004 0213795; 2004 0241745; 2006 0210567; 2007 0092504; 2007 0065427; and 2008 0025979 and U.S. Pat. No. 7,101,550. In some embodiments, humanized anti-PD-1, anti-PD-L1, and/or anti-PD-L2, anti-PD1 antibodies may be used.

CTLA4 (Cytotoxic T-Lymphocyte Antigen 4) is a CD28-family receptor expressed on CD4+ T cells. It binds the same ligands as CD28 (CD80 and CD86 on B cells and dendritic cells), but with higher affinity than CD28. In contrast to CD28, which enhances cell function when bound at the same time as the T cell receptor, CTLA4 inhibits T cell functioning. CTLA4 blockade releases inhibitory controls on T cell activation and proliferation, inducing antitumor immunity in both preclinical and early clinical trials (Quezada et al., 2006, J Clin Invest; 116: 1935-1945). The CTLA4 pathway is the subject of much interest (see, for example, U.S. Pat. No. 7,229,628). Blockade of CTLA4 with anti-CTLA4 antibodies can induce rejection of several types of established transplantable tumors in mice, including colon carcinoma, fibrosarcoma, prostatic carcinoma, lymphoma, and renal carcinoma (Leach et al., 1996, Science; 271:1734-1736; Kwon et al., 1997, Proc Natl Acad Sci USA; 94:8099-8103; Yang et al., 1997, Cancer Res; 57:4036-4041; Shrikant et al., 1999, Immunity; 11:483-493; and Sotomayor et al., 1999, Proc Natl Acad Sci USA; 96:11476-11481). Fully human anti-CTLA4 are being used in clinical trials with patients with melanoma or ovarian cancer (Hodi et al., 2003, Proc Natl Acad Sci USA; 100:4712-471717; Ribas et al., 2004, J Immunother; 27:354-367; and Phan et al., 2003, Proc Natl Acad Sci USA 100:8372-8377). Antibodies to block CTLA4 (such as Medarex MDX0101) are now in Phase II and II clinical trials (see, for example, Peggs et al., 2006, Curr Opin Immunol; 18:206-213). These studies collectively indicate the impact of CTLA4 blockade on tumor rejection (Korman et al., 2005, Curr Opin Investig Drugs; 6:582-591). However, adverse immune events have been documented in the initial clinical studies of CTLA4 blockade (Peggs et al., 2006, Curr Opin Immunol; 8:206-213). Currently, it is a problem that the anti-CTLA4 antibody only shows anti-tumor efficacy at doses that are toxic, due to development of nonspecific autoimmunity.

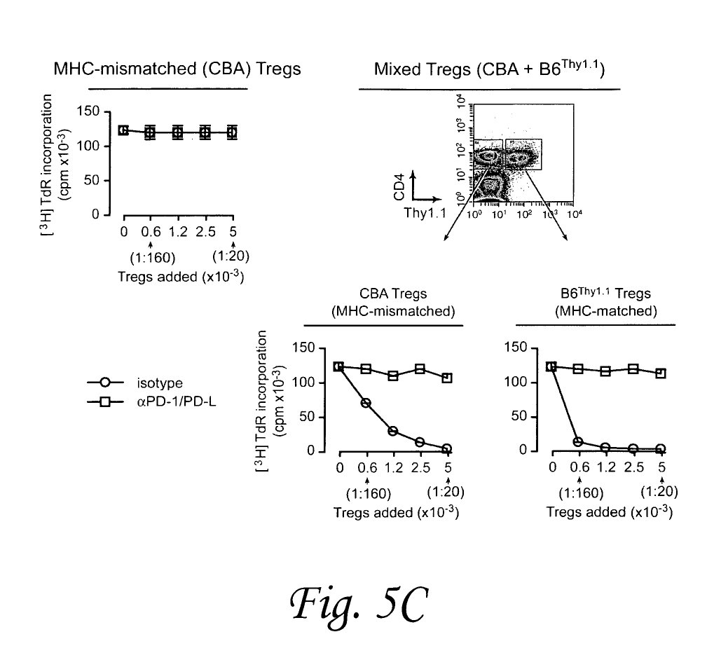

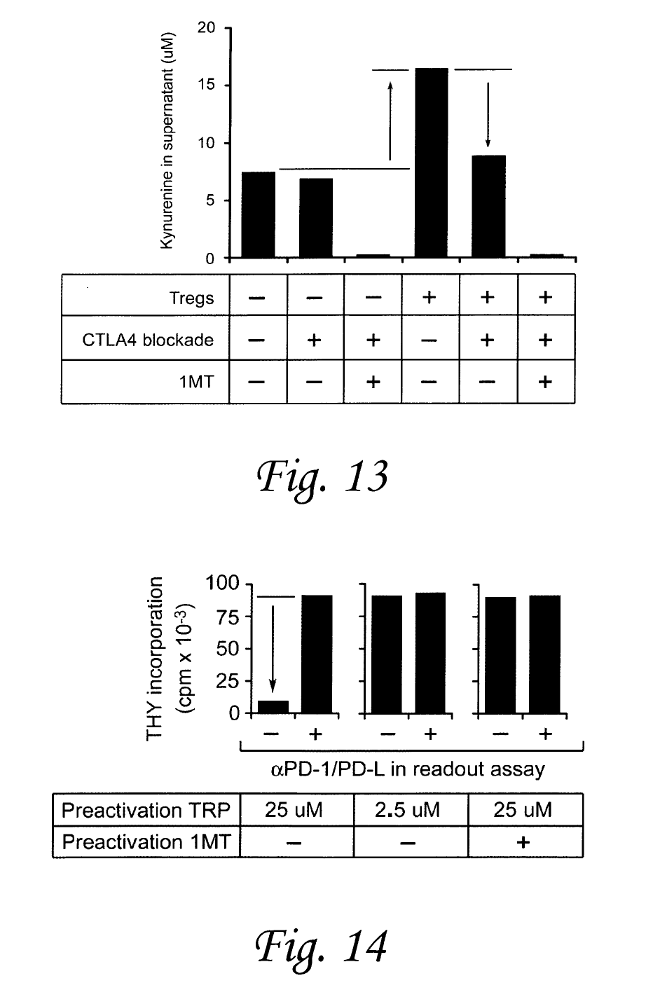

The present invention links the IDO-activated Treg pathway with the clinically-relevant CTLA4 pathway (see, for example, FIG. 5A and FIG. 13), addressing the problem of the toxicity observed with the administration of anti-CTLA4 antibody alone. The present invention demonstrates a benefit of combining anti-CTLA4 with an IDO inhibitor, such as 1MT, since by targeting a more specific step in the same pathway, the addition of an IDO inhibitor allows enhanced efficacy of lower-dose CTLA4 blockade, without the toxicity attendant on the high-dose CTLA4 blockade.

Inhibitors of the CTLA4 pathway include, but are not limited to antibodies, peptides, nucleic acid molecules (including, for example, an antisense molecule, a PNA, or an RNAi), peptidomimetics, small molecules, a soluble CTLA4 ligand polypeptide, or a chimeric polypeptide (for example, a chimeric CTLA4 ligand/immunoglobulin molecule). An antibody may be an intact antibody, an antibody binding fragment, or a chimeric antibody. A chimeric antibody may include both human and non-human portions. An antibody may be a polyclonal or a monoclonal antibody. An antibody may be a derived from a wide variety of species, including, but not limited to mouse and human. An antibody may be a humanized antibody. An antibody may be linked to another functional molecule, for example, another peptide or protein, a toxin, a radioisotype, a cytotoxic agent, cytostatic agent, a polymer, such as, for example, polyethylene glycol, polypropylene glycol or polyoxyalkenes. In some embodiments, a mixture or cocktail of various inhibitors of the CTLA4 pathway may be administered.

Any of a variety of antibodies may be used, including, but not limited to, any of those described herein and those commercially available from, for example, Medarex, Princeton, N.J. (Medarex MDX010); eBioscience, San Diego Calif. (clone 9H10) Abnova Corporation, Taipei City, Taiwan (CTLA4 monoclonal antibody (M08), clone 1F4 Catalog #: H00001493-M08 and CTLA4 polyclonal antibody (A01) Catalog #: H00001493-A01); RDI Division of Fitzgerald Industries Intl., Concord Mass. (mouse anti-human CTLA-4 antibodies clones BNI3.1 and ANC152.2 (J Immunol 151:3469; J Immunol; 155:1776; and J Immunol; 156:1047)); and BD Pharmingen (hamster anti-mouse CTLA4 IgG1; clone UC10-4F10-11; hybridoma HB-304T from ATCC).

Anti-CTLA4 antibodies include, but are not limited to, those taught in U.S. Pat. Nos. 7,311,910; 7,307,064; 7,132,281; 7,109,003; 7,034,121; 6,984,720; and 6,682,736. In some embodiments, one or more anti-CTLA4 antibodies may be humanized.

The methods of the present invention may also be administered to a patient for the treatment of cancer or an infection. The present invention includes methods of treating cancer or an infection in a subject by administering to the subject an inhibitor of IDO along with one or more inhibitors of the PD-1/PD-L pathway. The present invention includes methods of treating cancer or an infection in a subject by administering to the subject an inhibitor of IDO along with one or more inhibitors of the CTLA4 pathway. The present invention includes methods of treating cancer or an infection in a subject by administering to the subject an inhibitor of IDO along with one or more inhibitors of the PD-1/PD-L pathway and one or more inhibitors of the CTLA4 pathway.

Cancers to be treated by the present invention include, but are not limited to, melanoma, basal cell carcinoma, colorectal cancer, pancreatic cancer, breast cancer, prostate cancer, lung cancer (including small-cell lung carcinoma and non-small-cell carcinoma, leukemia, lymphoma, sarcoma, ovarian cancer, Kaposi's sarcoma, Hodgkin's Disease, Non-Hodgkin's Lymphoma, multiple myeloma, neuroblastoma, rhabdomyosarcoma, primary thrombocytosis, primary macroglobulinemia, small-cell lung tumors, primary brain tumors, stomach cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

The efficacy of treatment of a cancer may be assessed by any of various parameters well known in the art. This includes, but is not limited to, determinations of a reduction in tumor size, determinations of the inhibition of the growth, spread, invasiveness, vascularization, angiogenesis, and/or metastasis of a tumor, determinations of the inhibition of the growth, spread, invasiveness and/or vascularization of any metastatic lesions, determinations of tumor infiltrations by immune system cells, and/or determinations of an increased delayed type hypersensitivity reaction to tumor antigen. The efficacy of treatment may also be assessed by the determination of a delay in relapse or a delay in tumor progression in the subject or by a determination of survival rate of the subject, for example, an increased survival rate at one or five years post treatment. As used herein, a relapse is the return of a tumor or neoplasm after its apparent cessation, for example, such as the return of leukemia.

The present invention includes methods to enhance an immune response in a subject by administering an effective amount of an inhibitor of IDO along with one or more inhibitors of the PD-1/PD-L pathway and/or one or more inhibitors of the CTLA4 pathway. With such a method a vaccine may also be administered. Such a vaccine may be an anti-viral vaccine, such as, for example, a vaccine against HIV, or a vaccine against tuberculosis or malaria. The vaccine may be a tumor vaccine, including, for example, a melanoma, prostate cancer, colorectal carcinoma, or multiple myeloma vaccine. Dendritic cells (DC) have the ability to stimulate primary T cell antitumor immune responses. Thus, a tumor vaccine may include dendritic cells. Dendritic cell vaccines may be prepared, for example, by pulsing autologous DCs derived from the subject with synthetic antigens, tumor lysates, tumor RNA, or idiotype antibodies, by transfection of DCs with tumor DNA, or by creating tumor cell/DC fusions (Ridgway, Cancer Invest. 2003; 21:873-86). The vaccine may include one or more immunogenic peptides, for example, immunogenic HIV peptides, immunogenic tumor peptides, or immunogenic human cytomegalovirus peptides (such as those described in U.S. Pat. No. 6,251,399). The vaccine may include genetically modified cells, including genetically modified tumor cells or cell lines genetically modified to express granulocyte-macrophage stimulating factor (GM-CSF) (Dranoff, Immunol Rev. 2002; 188:147-54), or alpha(1,3)galatosyltransferase (see, for example, U.S. Pat. Nos. 5,879,675 and 6,361,775 and U/S/ Patent Application Serial Nos. 2007 0014775 and 2004 0191229). In some aspects of the invention, a vaccine may include an antigen that is the target of an autoimmune response.

The methods of the present invention may be used to treat infections, including, but not limited to, viral infections, infection with an intracellular parasite, and infection with an intracellular bacteria. Viral infections treated include, but are not limited to, infections with the human immunodeficiency virus (HIV) or cytomegalovirus (CMV). Intracellular bacterial infections treated include, but are not limited to infections with Mycobacterium leprae, Mycobacterium tuberculosis, Listeria monocytogenes, and Toxplasma gondii. Intracellular parasitic infections treated include, but are not limited to, Leishmania donovani, Leishmania tropica, Leishmania major, Leishmania aethiopica, Leishmania mexicana, Plasmodium falciparum, Plasmodium vivax, Plasmodium ovale, and Plasmodium malariae. The efficacy of treatment of an infection may be assessed by any of various parameters well known in the art. This includes, but is not limited to, a decrease in viral load, an increase in CD4⁺ T cell count, a decrease in opportunistic infections, eradication of chronic infection, and/or increased survival time.

The methods of the present invention may be used to treat chronic viral infections. Chronic viral infections that may be treated using the present methods include, but are not limited to, diseases caused by hepatitis C virus (HCV), human papilloma virus (HPV), cytomegalovirus (CMV), herpes simplex virus (HSV), Epstein-Barr virus (EBV), varicella zoster virus, coxsackie virus, and human immunodeficiency virus (HIV).

One or more additional therapeutic treatments may be administered along with the present methods of enhancing an immune response in a subject by administering an inhibitor of IDO along with one or more inhibitors of the PD-1/PD-L pathway and/or one or more inhibitors of the CTLA4 pathway, one or more additional therapeutic agents may be administered. As used herein, an additional therapeutic agent is not an IDO inhibitor, is not an inhibitor of the PD-1/PD-L pathway, and is not an inhibitor of the CTLA4 pathway. As used herein, an additional therapeutic agent is an agent whose use for the treatment of cancer, an infection, or an immune disorder is known the skilled artisan. Additional therapeutic treatments include, but are not limited to, surgical resection, radiation therapy, hormone therapy, vaccines, antibody based therapies, whole body irradiation, bone marrow transplantation, peripheral blood stem cell transplantation, the administration of chemotherapeutic agents (also referred to herein as “antineoplastic chemotherapy agent,” “antineoplastic agents,” or “antineoplastic chemotherapeutic agents”), cytokines, antiviral agents, immune enhancers, tyrosine kinase inhibitors, signal transduction inhibitors, antibiotic, antimicrobial agents, a TLR agonists, such as for example, bacterial lipopolysaccharides (LPS), one or more CpG oligonucleotides (ODN), metabolic breakdown products of tryptophan, inhibitors of a GCN2 kinase, and adjuvants.

A chemotherapeutic agent may be, for example, a cytotoxic chemotherapy agent, such as, for example, epidophyllotoxin, procarbazine, mitoxantrone, platinum coordination complexes such as cisplatin and carboplatin, leucovorin, tegafur, paclitaxel, docetaxol, vincristine, vinblastine, methotrexate, cyclophosphamide, gemcitabine, estramustine, carmustine, adriamycin (doxorubicin), etoposide, arsenic trioxide, irinotecan, epothilone derivatives, navelbene, CPT-11, anastrazole, letrazole, capecitabine, reloxafine, ifosamide, and droloxafine.

A chemotherapeutic agent may be, for example, an alkylating agent, such as, for example, nitrogen mustards (such as chlorambucil, cyclophosphamide, ifosfamide, echlorethamine, melphalan, and uracil mustard), aziridines (such as thiotepa), methanesulphonate esters (such as busulfan), nitroso ureas (such as carmustine, lomustine, and streptozocin), platinum complexes (such as cisplatin and carboplatin), and bioreductive alkylators (such as mitomycin, procarbazine, dacarbazine and altretamine), ethylenimine derivatives, alkyl sulfonates, triazenes, pipobroman, temozolomide, triethylene-melamine, and triethylenethiophosphoramine.

A chemotherapeutic agent may be an antimetabolite, such as, for example, a folate antagonist (such as methotrexate and trimetrexate), a pyrimidine antagonist (such as fluorouracil, fluorodeoxyuridine, CB3717, azacitidine, cytarabine, gemcitabine, and floxuridine), a purine antagonist (such as mercaptopurine, 6-thioguanine, fludarabine, and pentostatin), a ribonucleotide reductase inhibitor (such as hydroxyurea), and an adenosine deaminase inhibitor.

A chemotherapeutic agent may be a DNA strand-breakage agent (such as, for example, bleomycin), a topoisomerase II inhibitor (such as, for example, amsacrine, dactinomycin, daunorubicin, idarubicin, mitoxantrone, doxorubicin, etoposide, and teniposide), a DNA minor groove binding agent (such as, for example, plicamydin), a tubulin interactive agent (such as, for example, vincristine, vinblastine, and paclitaxel), a hormonal agent (such as, for example, estrogens, conjugated estrogens, ethinyl estradiol, diethylstilbesterol, chlortrianisen, idenestrol, progestins (such as hydroxyprogesterone caproate, medroxyprogesterone, and megestrol), and androgens (such as testosterone, testosterone propionate, fluoxymesterone, and methyltestosterone)), an adrenal corticosteroid (such as, for example, prednisone, dexamethasone, methylprednisolone, and prednisolone), a leutinizing hormone releasing agent or gonadotropin-releasing hormone antagonist (such as, for example, leuprolide acetate and goserelin acetate), an antihormonal agent (such as, for example, tamoxifen), an antiandrogen agent (such as flutamide), an antiadrenal agent (such as mitotane and aminoglutethimide), and a natural product or derivative thereof (such as, for example, vinca alkaloids, antibiotics, enzymaes and epipodophyllotoxins, including, for example vinblastine, vincristine, vindesine, bleomycin, dactinomycin, daunorubicin, doxorubicin, epirubicin, idarubicin, ara-C, paclitaxel, mithramycin, deoxyco-formycin, mitomycin-C, L-asparaginase, and teniposide.

Antiviral agents include, but are not limited to, acyclovir, gangcyclovir, foscarnet, ribavirin, and antiretrovirals. Antiretrovirals include, for example, nucleoside analogue reverse transcriptase inhibitors (such as, for example, azidothymidine (AZT), didanosine (ddI), zalcitabine (ddC), stavudine (d4T), lamivudine (3TC), abacavir (1592U89), adefovir dipivoxil(bis(POM)-PMEA), lobucavir (BMS-180194), BCH-10652, emitricitabine ((−)-FTC), beta-L-FD4, DAPD, ((−)-beta-D-2,6,-diamino-purine dioxolane), and lodenosine (FddA)), non-nucleoside reverse transcriptase inhibitors (such as, for example, nevirapine, delaviradine, efavirenz, PNU-142721, AG-1549, MKC-442, and (+)-calanolide A (NSC-675451)), nucleotide analogue reverse transcriptase inhibitors, protease inhibitors (such as, for example, saquinavir, ritonavir, indinavir, nelfnavir, amprenavir, lasinavir, DMP-450, BMS-2322623, ABT-378, and AG-1549) and other antivirals (such as, for example, hydroxyurea, ribavirin, IL-2, IL-12, and pentafuside).

Cytokines include, but are not limited to, IL-1α, IL-1β, IL-2, IL-3, IL-4, IL-6, IL-8, IL-9, IL-10, IL-12, IL-13, IL-15, IL-18, IL-19, IL-20, IFN-α, IFN-β, IFN-γ, tumor necrosis factor (TNF), transforming growth factor-β (TGF-β), granulocyte colony stimulating factor (G-CSF), macrophage colony stimulating factor (M-CSF), granulocyte-macrophage colony stimulating factor (GM-CSF), and or Flt-3 ligand.

Vaccines include, but are not limited to, vaccines against various infectious diseases, anti-tumor vaccines and anti-viral vaccines. Antitumor vaccines include, but are not limited to, peptide vaccines, whole cell vaccines, genetically modified whole cell vaccines, recombinant protein vaccines or vaccines based on expression of tumor associated antigens by recombinant viral vectors.

Antibody therapeutics, include, for example, trastuzumab (Herceptin) and antibodies to cytokines, such as IL-10 and TGF-β.

Signal transduction inhibitors (STI) include, for example, bcr/abl kinase inhibitors such as, for example, STI 571 (Gleevec), epidermal growth factor (EGF) receptor inhibitors such as, for example, kinase inhibitors (Iressa, SSI-774) and the antibody C225, her-2/neu receptor inhibitors such as, for example, trastuzumab and farnesyl transferase inhibitors (FTI) such as, for example, L-744,832, inhibitors of Akt family kinases or the Akt pathway, such as, for example, rapamycin, cell cycle kinase inhibitors such as, for example, flavopiridol and UCN-01, and phosphatidyl inositol kinase inhibitors such as, for example, LY294002.

Inhibitors of GCN2 prevent the development or reactivation of Tregs by IDO. The protein kinase GCN2 (also referred to as “General Control Nonderepressible 2,” “eIF2AK4,” and “eukaryotic translation initiation factor 2 alpha kinase 4”) has been shown to play a role in the induction of proliferative arrest and anergy of CD8⁺ T cells in the presence of IDO+ DCs (see Munn et al., 2005, Immunity; 22:1-10). Specifically, Munn et al. demonstrated that in order for IDO to mediate the proliferative arrest and anergy of effector T cells, the cells need GCN2. Thus, GCN2 is downstream in the pathway of IDO effects and inhibiting the function of GCN2 with an inhibitory agent should result in blockade of the inhibitory effect of IDO on the effector T cells. The expression of IDO by human DCs induces the differentiation of naive CD4+ T cells into Tregs, and this is mediated by Trp metabolites such as Kynurenine. It has also been shown that the combined effects of Trp depletion and Trp catabolites induces naïve T cells to acquire a regulatory phenotype, and that this mechanism was mediated by GCN2, since T cells from GCN2 knockout animals did not develop the regulatory phenotype (Fallarino et al., 2006, J Immunol; 176:6752-6761). Targeting GCN2 kinase with inhibitory agents can serve as an alternative to direct IDO inhibition (see, also, Muller and Scherle, 2006, Nature Reviews Cancer; 6:613). Thus, GCN2 has been implicated in mediating the effects of IDO in various cell types, including, but not limited to, effector CD8⁺ T cells and naive CD4⁺ T cells. Inhibitors of GCN2 may be used to bypass or replace the need for IDO inhibitors. The present invention includes any of the various methods described herein, in which an IDO inhibitor supplemented with a GCN2 inhibitor. Candidate GCN2 inhibitors, include, for example, a GCN2 blocking peptide, an antibody to GCN2 (both commercially available, for example, from Bethyl, Inc., Montgomery, Tex.) and small molecule inhibitors (including, for example, those discussed by Muller and Scherle, 2006, Nature Reviews Cancer; 6:613).

As used herein “treating” or “treatment” includes both therapeutic and prophylactic treatments. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis. The findings of the present invention can be used in methods that include, but are not limited to, methods for treating cancer, methods to treat an infections, methods to increase an immune responses, methods to reduce immunosuppression mediated by regulatory T cells, and methods to increase or stimulate T cell mediated immune responses.

The agents of the present invention can be administered by any suitable means including, but not limited to, for example, oral, rectal, nasal, topical (including transdermal, aerosol, buccal and sublingual), vaginal, parenteral (including subcutaneous, intramuscular, intravenous and intradermal), intravesical, or injection into or around the tumor.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, intraperitoneal, and intratumoral administration. In this connection, sterile aqueous media that can be employed will be known to those of skill in the art in light of the present disclosure (see for example, “Remington's Pharmaceutical Sciences” 15th Edition). Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA.

For enteral administration, the inhibitor may be administered in a tablet or capsule, which may be enteric coated, or in a formulation for controlled or sustained release. Many suitable formulations are known, including polymeric or protein microparticles encapsulating drug to be released, ointments, gels, or solutions which can be used topically or locally to administer drug, and even patches, which provide controlled release over a prolonged period of time. These can also take the form of implants. Such an implant may be implanted within the tumor.

Therapeutically effective concentrations and amounts may be determined for each application herein empirically by testing the compounds in known in vitro and in vivo systems, such as those described herein, dosages for humans or other animals may then be extrapolated therefrom.

It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

An agent of the present invention may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions and methods.

With the present invention, the stimulation or inhibition of an immune response may be measured by any of many standard methods well known in the immunological arts. As used herein, a mixed leukocyte response (MLR) is a well-known immunological procedure, for example, as described in the examples herein. As used herein, T cell activation by an antigen-presenting cell is measured by standard methods well known in the immunological arts. As used herein, a reversal or decrease in the immunosuppressed state in a subject is as determined by established clinical standards. As used herein, the improved treatment of an infection is as determined by established clinical standards. The determination of immunomodulation includes, but is not limited to, any of the various methods as described in the examples herein.

With the methods of the present invention, the efficacy of the administration of one or more agents may be assessed by any of a variety of parameters well known in the art. This includes, for example, determinations of an increase in the delayed type hypersensitivity reaction to tumor antigen, determinations of a delay in the time to relapse of the post-treatment malignancy, determinations of an increase in relapse-free survival time, determinations of an increase in post-treatment survival, determination of tumor size, determination of the number of reactive T cells that are activated upon exposure to the vaccinating antigens by a number of methods including ELISPOT, FACS analysis, cytokine release, or T cell proliferation assays.

As used herein, the term “subject” includes, but is not limited to, humans and non-human vertebrates. Non-human vertebrates include livestock animals, companion animals, and laboratory animals. Non-human subjects also include non-human primates as well as rodents, such as, but not limited to, a rat or a mouse. Non-human subjects also include, without limitation, chickens, horses, cows, pigs, goats, dogs, cats, guinea pigs, hamsters, mink, and rabbits. As used herein, the terms “subject,” “individual,” “patient,” and “host” are used interchangeably. In preferred embodiments, a subject is a mammal, particularly a human.

As used herein “in vitro” is in cell culture and “in vivo” is within the body of a subject.

As used herein, the term “pharmaceutically acceptable carrier” refers to one or more compatible solid or liquid filler, diluents or encapsulating substances which are suitable for administration to a human or other vertebrate animal.

As used herein, the term “isolated” as used to describe a compound shall mean removed from the natural environment in which the compound occurs in nature. In one embodiment isolated means removed from non-nucleic acid molecules of a cell.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

In some therapeutic embodiments, an “effective amount” of an agent is an amount that results in a reduction of at least one pathological parameter. Thus, for example, in some aspects of the present invention, an effective amount is an amount that is effective to achieve a reduction of at least about 10%, at least about 15%, at least about 20%, or at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%, compared to the expected reduction in the parameter in an individual not treated with the agent.

The present invention is illustrated by the following examples. It is to be understood that the particular examples, materials, amounts, and procedures are to be interpreted broadly in accordance with the scope and spirit of the invention as set forth herein.

### Examples

### Example 1

### Plasmacytoid Dendritic Cells from Mouse Tumor-Draining Lymph Nodes Directly Activate Mature Tregs Via indoleamine 2,3-dioxygenase

A subset of DCs in murine tumor-draining lymph nodes (TDLNs) can express high levels of the tryptophan-degrading enzyme indoleamine 2,3-dioxygenase (IDO) (Munn et al., 2004, J Clin Invest; 114:280-290). In other settings, IDO has been shown to contribute to maternal tolerance toward the allogeneic fetus, regulation of autoimmune disorders, and creation of tolerance to transplanted tissues (Munn et al., 1998, Science; 281:1191-1193; Gurtner et al., 2003, Gastroenterology; 125:1762-1773; and Liu et al., 2006, FASEB J; 20:2384-2386). Transfection of IDO into tumor cells protects them from immune-mediated rejection (Uyttenhove et al., 2003, Nat Med; 9:1269-1274), while inhibiting IDO in tumor-bearing hosts allows conventional chemotherapy to disrupt tolerance toward established tumors and trigger anti-tumor immune responses (Muller et al., 2005, Nat Med; 11:312-319 and Hou et al., 2007, Cancer Res; 67:792-801). Thus, IDO is an important tolerogenic mechanism in patients with cancer (Munn and Mellor, 2007, J Clin Invest; 117:1147-1154).

In vitro studies of IDO⁺ DCs from murine tumor-draining lymph nodes (TDLNs) have shown that these cells are potently and dominantly suppressive for T cell activation (Munn et al., 2004, J Clin Invest; 114:280-290; Hou et al., 2007, Cancer Res; 67:792-801; and Munn et al., 2005, Immunity; 22:633-642). Even a small minority of IDO⁺ DCs are capable of inhibiting all T cell responses in culture, including dominant inhibition of T cells responding to antigens presented by other, nonsuppressive APCs (Munn et al., 2004, J Clin Invest; 114:280-290). In vivo, pharmacologic activation of the IDO pathway systemically can completely inhibit clonal expansion of large numbers of alloreactive T cells (Mellor et al., 2003, J Immunol. 171:1652-1655). However, the number of IDO DCs that become activated in spleen or TDLNs is tiny (<1% of total cells, and typically <25% of total DCs), and it is unclear how the effects of IDO could create such potent and dominant immunosuppression.

Recently it has been shown that IDO can bias naive CD4⁺ T cells to differentiate into Foxp3⁺ regulatory T cells (Tregs) in vitro (Fallarino et al., 2006, J Immunol; 176:6752-6761). This important finding thus linked IDO to the potent Treg system, which is known to be a key mechanism of immunosuppression in tumor bearing hosts (Zou, 2006, Nat Rev Immunol; 6:295-307). However, de novo differentiation of Tregs from naive precursor cells is a slow process, requiring many days; whereas we knew from in vitro studies that IDO created dominant suppression within hours, prior to the first cell division of the suppressed T cells (Munn et al., 2005, Immunity; 22:633-642 and Munn et al., 1999, J Exp Med; 189:1363-1372). Therefore, there exists a pathway by which IDO could directly activate the latent suppressor function of mature, pre-existing Tregs this pathway would be active in TDLNs in vivo. The findings of this example can also be found in the recently published Sharma et al., “Plasmacytoid dendritic cells from mouse tumor-draining lymph nodes directly activate mature Tregs via indoleamine 2,3-dioxygenase,” J Clin Invest. 2007 September; 117(9):2570-82 (published online Aug. 16m, 2007; doi 10.1172/JCI31911) and its accompanying supplemental online material.

### Methods

Mice and tumors. Animal studies were approved by the Institutional Animal Care and Use Committee of the Medical College of Georgia. TCR-transgenic OT-I mice (CD8⁺, B6 background, recognizing the SIINFEKL (SEQ ID NO:1) peptide of ovalbumin (OVA) on H2K^{b} (Hogquist et al., 1994, Cell; 76:17-27)) and B6.PL-Thy1a/CyJ mice (congenic for the B6 background but bearing the Thy 1.1 allele) were purchased from Jackson Laboratories (Bar Harbor, Me.). GCN2-KO mice (B6 background) were a generous gift from the laboratory of David Ron (New York University School of Medicine) and have been previously described (Munn et al., 2005, Immunity; 22:633-642). A1 mice (CBA background, recognizing an H-Y peptide presented on IE^{k}) (Zelenika et al., 1998, J Immunol; 161:1868-1874), BM3 (CBA background, recognizing H2K^{b} as an allo-antigen (Tarazona et al., 1996, Int Immunol; 8:351-358)) and IDO-KO mice (B6 and CBA backgrounds (Mellor et al., 2003, J Immunol. 171:1652-1655 and Baban et al., 2005, Int Immunol; 17:909-919)) have been described.

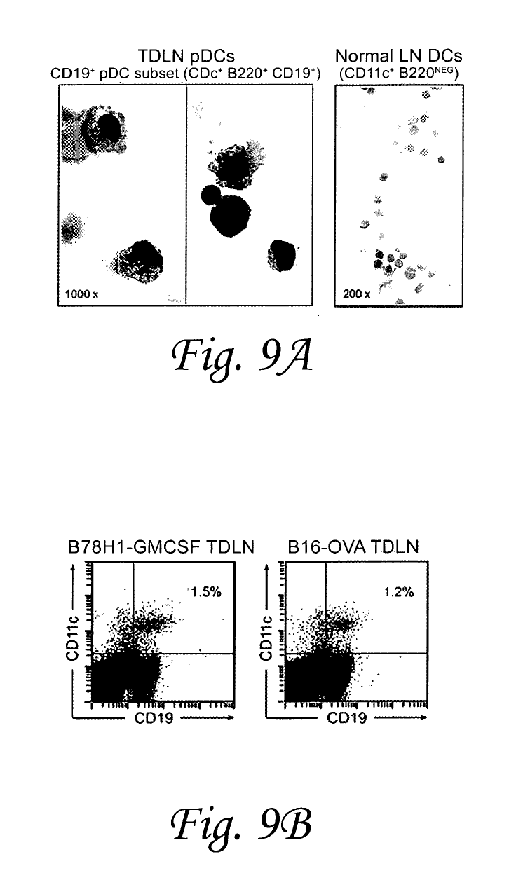

Tumor implantation is described in more detail below. Cell lines used were B78H1-GMCSF (a subline of B16 transfected with GMCSF (Huang et al., 1994, Science; 264:961-965), as used in previous studies of IDO⁺ pDCs (Munn et al., 2004, J Clin Invest; 114:280-290 and Munn et al., 2005, Immunity; 22:633-642 9)); the B16F10 subline of B16 (ATCC); and B16-OVA (parental B16F10 transfected with full-length chicken ovalbumin, clone MO4 (Falo et al., 1995, Nat Med; 1:649-653)). The use of OVA as a model tumor antigen was informative in this system, since the goal was to detect the suppression of immune response to tumor antigens; thus, a strong nominal antigen was an advantage. IDO⁺ pDCs and activated Tregs found in TDLNs of all three tumor lines were similar. As in previous studies (Munn et al., 2004, J Clin Invest; 114:280-290 and Munn et al., 2005, Immunity; 22:633-642), most experiments requiring sorted pDCs used the B78H1-GMCSF tumors, because these gave the highest yield of pDCs (see FIG. 9). However, pDCs from tumors without GMCSF gave similar functional results, and all key findings were confirmed with tumors with and without GMCSF.

1-methyl-D-tryptophan (catalog #45,248-3, Sigma) was prepared as described (Munn et al., 2005, Immunity; 22:633-642) and used at a final concentration of 200 uM. Delivery of 1MT by sustained-release subcutaneous pellets (5 mg/day) was as described (Hou et al., 2007, Cancer Res; 67:792-801). For oral delivery, 1MT was added to drinking water at 2 mg/ml. Recombinant mouse IL-2 (R&D Systems) was used at 10 ng/ml. Blocking antibodies against PD-L1/B7-DC (clone MIH7) (Tsushima et al., 2003, Eur J Immunol; 33:2773-2782), PD-L2 (TY25) (Yamazaki et al., 2002, J Immunol; 169:5538-5545), and PD-1 (J43) (Agata et al., 1996, Int Immunol; 8:765-772) were used as a cocktail at 50 μg/ml each (or rat IgG1 isotype control).

Anti-CTLA4 antibody (clone 9H10, used at 10 μg/ml) and rat anti-IL-10-receptor antibody (used at 100 μg/ml, clone 1B1.3a) were from BD-Biosciences; anti-mouse I-A^{b} (used at 100 μg/ml) and IgM isotype control were from Southern Biotech; chicken anti-TGF-β/β2/β3 (MAB1835, used at 100 μg/ml) was from R&D Systems.

Ex vivo Treg assays. Tregs (CD4⁺CD25⁺) were sorted from 2-4 pooled TDLNs and added directly to readout assays containing 1×10⁵ CD4⁺ A1 cells, 2×10³ CD11c⁺ DCs from CBA spleen, and 100 nM H-Y peptide (REEALHQFRSGRKPI) (SEQ ID NO:2). All cultures were performed in V-bottom wells. For both Tregs and pDCs, it was important to perform sorts rapidly, collect cells in complete medium on ice, and transfer them promptly into culture, in order to preserve viability and function.

Treg activation culture and readout assays. Sorting of pDCs from TDLNs was performed as described (Munn et al., 2004, J Clin Invest; 114:280-290 and Munn et al., 2005, Immunity; 22:633-642). The pDC fraction (CD11c⁺B220⁺) was sorted from 2-6 pooled TDLNs (day 7-11 of tumor growth) and collected in medium on ice. Pre-activation cultures contained 2×10³ pDCs, 1×10⁵ sorted CD8⁺ OT-I cells, 100 nM SIINFEKL peptide (SEQ ID NO:1), and 5×10³ sorted CD4⁺CD25⁺ Tregs from spleens of B6 mice without tumors. All cultures received a feeder layer of 1×10⁵ T cell-depleted spleen cells (CD4^{NEG}CD8^{NEG}), as described below. For anti-CD3-induced activation, the same cultures received 200 uM 1MT to block IDO plus 0.1 ug/ml anti-CD3 mAb (clone 145-2C11, BD-Pharmingen) and 10 ng/ml IL-2. IL-2 was routinely added to the anti-CD3 pre-activation cultures, although this did not have any further enhancing effect on suppressor activity over anti-CD3 alone, presumably because adequate IL-2 was contributed by the activating OT-I cells (Thornton et al., 2004, J Immunol; 172:6519-6523). After two days, cultures were harvested, stained for CD4, and Tregs isolated by sorting for CD4⁺ cells. Preliminary studies showed that sorting on either total CD4⁺ Tregs or the CD4⁺CD62L^{H1} subset of Tregs gave equivalent results, so the total CD4⁺ Treg population was routinely used. Re-sorted Tregs were added to readout assays containing 1×10⁵ A1 cells, 2×10³ CD11c⁺ DCs from CBA spleen (or 5×10⁴ B cells, as CD11c^{NEG} B220⁺ spleen cells), plus H-Y peptide. For assays performed in transwells, Multiwell 96-well insert plates (1 uM pore size, BD-Falcon) were used and the number of cells in all groups was doubled.

DC and T cell adoptive transfer. The DC adoptive transfer model, and adoptive transfer of CFSE-labeled T cells, have been previously described (Munn et al., 2004, J Clin Invest; 114:280-290 and Munn et al., 2005, Immunity; 22:633-642), and detailed methods are given below.

Isolation of tumor-draining lymph node DCs. Tumors were initiated using 1×10⁶ B78H1-GM-CSF cells, a sub-line of B16 melanoma transfected with GM-CSF, that recruits many DCs into TDLNs (Huang et al., 1994, Science; 264:961-965 and Borrello et al., 1999, Hum Gene Ther; 10:1983-1991. This model was used in previous reports, because it recruits a large number of IDO⁺ pDCs in TDLNs (Munn et al., 2004, J Clin Invest; 114:280-290 and Munn et al., 2005, Immunity; 22:633-642). To rule out any effect of the GMCSF transgene, all key findings were also replicated with B16F10 and/or B16F10-OVA, with similar results. The advantage of the GMCSF-transfected tumor cells was that they yielded more cells from each TDLN, thus reducing the number of mice required. FACS analysis showed that the pDCs recruited by B78H1 GM-CSF tumors were phenotypically identical to those recruited by B16F10 tumors (Munn et al., 2004, J Clin Invest; 114:280-290).

Tumors were implanted in the thigh of either B6 mice or IDO-KO mice on the B6 background, positioned so as to drain to the inguinal LN as described (Munn et al., 2004, J Clin Invest; 114:280-290). Inguinal TDLNs were removed for cell sorting on day 11-14. IDO⁺ DCs were enriched using high-speed MoFlo cell sorting for CD11c⁺ B220⁺ cells as described (Munn et al., 2004, J Clin Invest; 114:280-290). This fraction included the specific subset of CD 19⁺ CD11c⁺ B220⁺ cells that have shown to comprise virtually all of the IDO-mediated suppression in TDLNs (Munn et al., 2004, J Clin Invest; 114:280-290). While CD19 is usually considered a marker for B cells, it has been shown that a subset of pDCs also expresses B-lineage markers (Pelayo et al., 2005, Blood; 105:4407-4415 and Corcoran et al., 2003, J Immunol; 170:4926-4932). It has been shown that the CD19⁺ “IDO-competent” DC population in spleen mediates IDO-induced suppression in a number of settings (Baban et al., 2005, Int Immunol; 17:909-919 and Mellor et al., 2005, J Immunol; 175:5601-5605). The viability and recovery of the IDO⁺ subset was improved during sorting if the CD19⁺ cells were isolated as part of the total pDC fraction (CD11c⁺B220⁺ cells).

In this example, it was irrelevant whether the IDO⁺ pDCs were strictly purified; it was only necessary that a population of IDO⁺ cells be present. Therefore, the total pDC fraction (CD11c⁺ B220⁺) was used as the source IDO+ cells, just as in previous studies (Munn et al., 2004, J Clin Invest; 114:280-290; Munn et al., 2005, Immunity; 22:633-642; and Hou et al., 2007, Cancer Res; 67:792-801).

Feeder layer. Sorted IDO⁺ pDCs required survival factors to maintain viability and function in vitro. As a feeder layer we added T cell-depleted spleen cells (1×10⁵ sorted CD4^{NEG}CD8^{NEG} cells) to all assays. This feeder layer was required, but was entirely nonspecific, and could be derived from any host regardless of MHC haplotype (H2^{b}, H2^{k} or H2^{d}), strain background (B6, CBA, Balb/c or 129), or genotype (GCN2-KO, IDO-KO or Foxp3-KO/scurfy mice). The feeder layer could be fully replaced by a cocktail of recombinant cytokines, comprising mouse IFNα (1000 U/ml, PBL Biomedical Laboratories, Piscataway, N.J.)+mouse IL-10 (100 ng/ml, R&D Systems)+human TGF-β1 (cross-reactive with mouse, 10 ng/ml, R&D Systems). When recombinant cytokines were used, Treg activation still required the presence of IDO⁺ pDCs, and was still abrogated by 1MT. Thus, the function of the feeder layer was purely supportive.

FACS staining. Antibodies were from BD-Pharmingen unless otherwise noted. Anti-mouse CD25-APC conjugate (clone PC61, cat. #17-0251-81) was from eBioscience; this conjugate gave brighter signal and better separation of CD25⁺ cells than other conjugates from other suppliers. For intracellular staining of CHOP, live cells were first blocked for 10 minutes with mouse Fc Block (BD Pharmingen) in 10% fetal calf serum medium, stained with anti-CD4-FITC for 30 minutes on ice, washed with PBS, then fixed and permeablized for 20 minutes in 250 μl Cytoperm/Cytofix solution (BD Pharmingen) on ice. All subsequent staining and wash steps were in BD Permwash solution. Fixed cells were stained with 1:100 dilution of monoclonal anti-gadd153/CHOP (sc-7351, Santa Cruz Biotechnology), washed, and stained with secondary monoclonal rat anti-mouse-IgG1-PE (#550083, BD Biosciences). This secondary antibody was selected because it did not cross-react with surface immunoglobulin on mouse B cells, which was important for staining mouse T cells using a mouse primary antibody. For Foxp3 staining, anti-Foxp3-PE antibody (clone FJK-16s) was obtained from eBioscience and used per the manufacturer's protocol. For Foxp3 staining, assays omitted A1 bystander cells and Tregs were identified by CD4 expression, as for CHOP staining. Biotinylated anti-PD-L1 (clone MIH5) and anti-PD-L2 (clone TY25) were purchased from eBioscience.

DC adoptive transfer. The DC adoptive-transfer model has been previously described (Munn et al., 2004, J Clin Invest; 114:280-290 and Munn et al., 2005, Immunity; 22:633-642). Briefly, total CD11c⁺ DCs were sorted from TDLNs, pulsed with SIINFEKL peptide (SEQ ID NO:1), and 5×10⁴ DCs injected subcutaneously into each anteriomedial thigh of recipient mice. Recipients had been pre-loaded one day before DC injection with 5×10⁶ sorted CD8⁺ OT-I cells. After four days, the inguinal LNs draining the site of DC injection were removed, and CD4⁺CD25⁺ Tregs were isolated by cell sorting.

CFSE labeling and T cell adoptive transfer. Mice were implanted with B16-OVA tumors. On day 7-8, sorted CD8⁺ T cells from WT OT-I or from OT-I bred onto the GCN2-KO background, were labeled with CFSE, and 5×10⁶ cells injected i.v., as described (Munn et al., 2005, Immunity; 22:633-642). Mice received either vehicle control, or 1MT at a concentration of 2 mg/ml in drinking water. After four days, the TDLNs and contralateral LNs (CLN) were harvested and stained for 1B11 (BD-Pharmingen) vs. CD8.

Statistical analysis. Individual thymidine in corporation assays were performed in triplicate or quadruplicate wells for each data point, and bars for these analyses error bars in the figures indicate SD of replicate wells. Multiple treatment groups in each experiment were compared by ANOVA. In cases where one representative experiment of several is shown, each independent replicate experiment showed comparable statistical significance between the same groups by ANOVA. Where multiple experiments were combined for analysis, raw thymidine incorporation counts were normalized to the control (wells without Tregs) in each experiment to permit comparison across multiple experiments. For these analyses, the error bars in the figures indicate the SD of the pooled data.

### Results

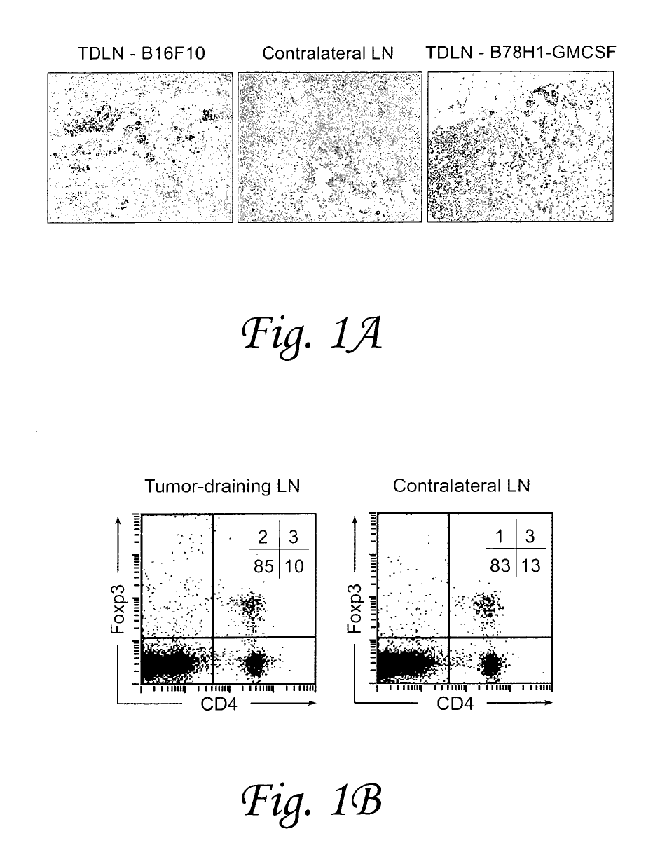

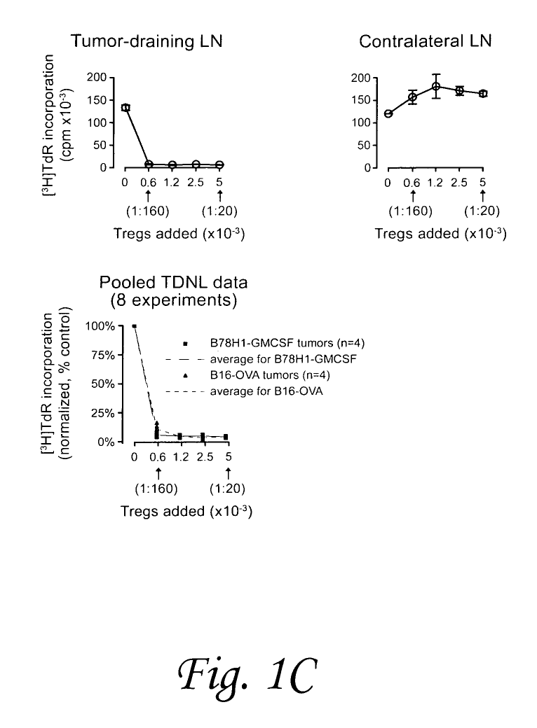

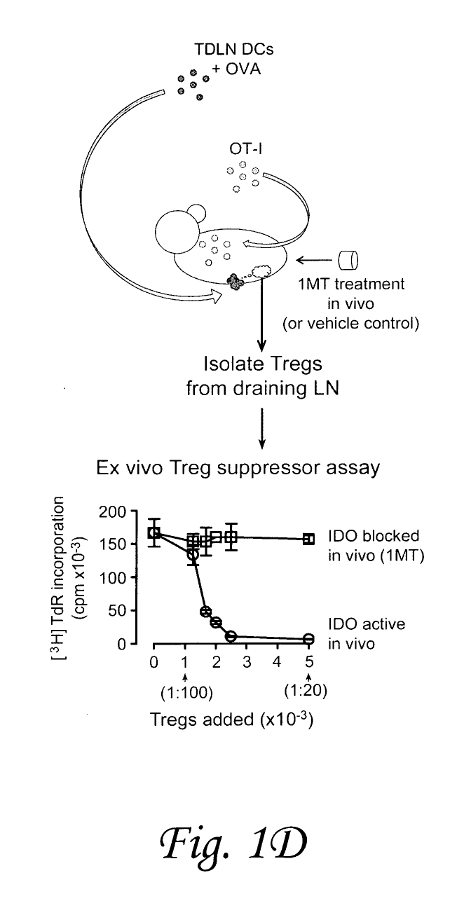

FIG. 1 shows Treg activation by DCs from TDLNs. FIG. 1A shows immunohistochemical staining of contralateral LN and TDLNs from mice with B16F10 and B78H1-GMCSF tumors. It has been previously shown that this CD19⁺ subset of pDCS contains essentially all of the functional IDO-mediated suppressor activity in these TDLNs. Control cells (the non-plasmacytoid DC fractions of normal LNs) showed minimal IDO staining. Staining controls (neutralization of the primary antibody with excess of the immunizing peptide) were all negative). In FIG. 1B, TDLNs and contralateral LNs were stained for CD4 versus intracellular Foxp3. Quadrant percentages are shown. FIG. 1B is representative of six experiments using B16-OVA and B78H1-GMCSF. In FIG. 1C, Tregs (CD4⁺CD25⁺) from TDLNs or contralateral LNs were sorted and added to readout assays comprising 1×10⁵ A1 T cells plus CBA DCs plus H-Y peptide. Proliferation ([³H]thymidine incorporation) is shown for a representative experiment, with the ratio of Tregs to A1 cells shown below the axis (bars show SD of replicate wells). The lower graph shows data from eight independent experiments using the tumor types shown (cpm were normalized to the proliferation in control assays receiving no Tregs, to permit comparison across experiments). In FIG. 1D, CD11c⁺ DCs were harvested from TDLNs, pulsed with OVA peptide, and injected subcutaneously into recipient mice pre-loaded with OT-I T cells. One group of mice received implantable sustained-release 1MT pellets at 5 mg/day (“IDO blocked”), while the other received vehicle control pellets (“IDO active”). After four days, the LNs draining the site of DC injection were harvested and the Tregs sorted and tested in vitro for spontaneous suppressor activity in readout assays (A1 T cells plus CBA DCs). FIG. 1D is representative of three experiments; bars show SD of replicate wells.

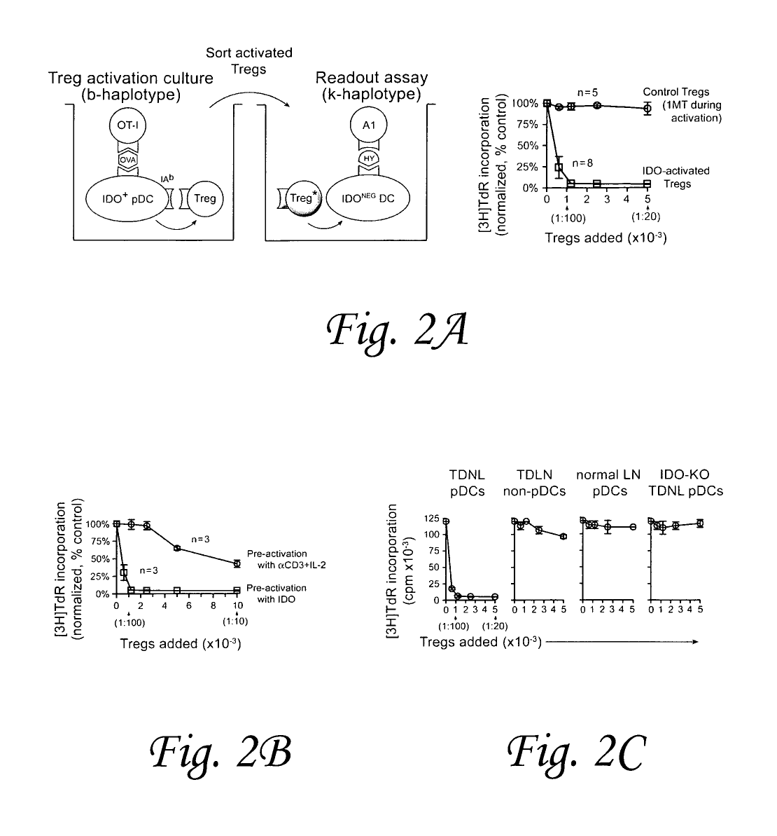

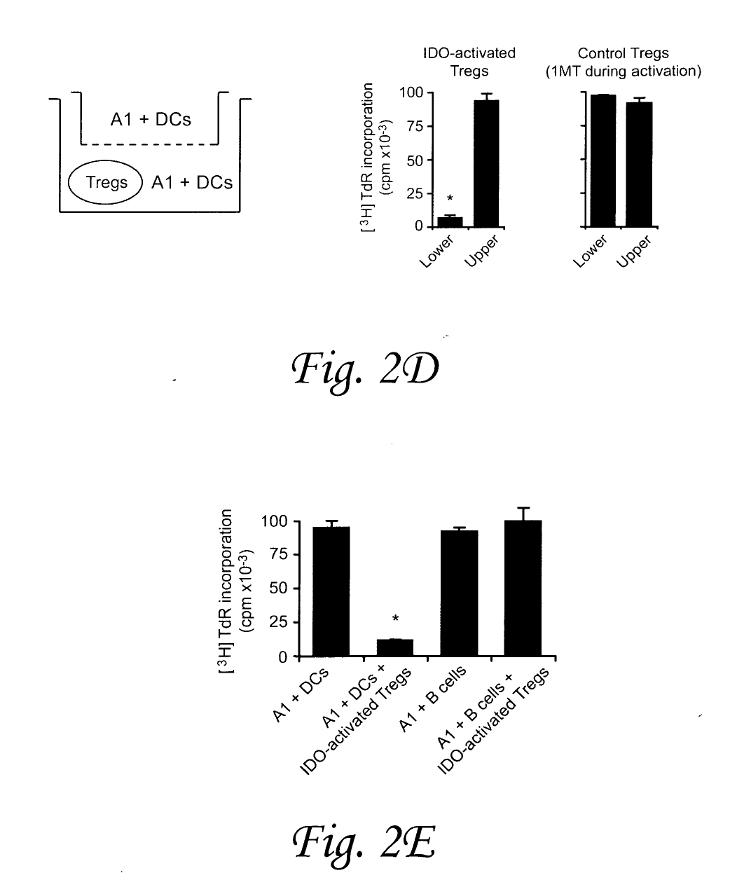

FIG. 2 shows activation of Tregs by IDO in vitro. In FIG. 2A, resting Tregs were co-cultured with TDLN pDCs plus OT-I T cells plus feeder cells. After two days the Tregs were re-sorted and added to readout assays (A1 T cells+CBA DCs). As controls, Tregs were activated in identical cultures with 1MT added to block IDO activity. Graph shows the mean of 5-8 pooled experiments, using pDCs from B78H1-GMCSF and B16-OVA tumors; bars show SD. In FIG. 2B, Tregs were activated as above, or in identical cultures containing 1MT to block IDO plus anti-CD3 mAb plus IL-2 to activate the Tregs. After two days, Tregs were re-sorted and tested in readout assays. Data-points show the means for pooled values from three independent experiments. In FIG. 2C, Tregs were activated in co-cultures as above, with the APCs being either TDLN pDCs; non-pDC fraction from the same TDLN (CD11c⁺B220^{NEG}); pDCs from mice without tumors; or TDLN pDCs from IDO-KO mice. Graphs show one of 3-4 similar experiments for each group (bars show SD of replicate wells). In FIG. 2D, Tregs were activated with TDLNs pDCs, as above, with or without 1MT. Tregs were resorted and added to readout assays in the lower chamber of transwell plates; upper chambers received readout assays without Tregs. Thymidine incorporation was measured separately in each chamber. One of three experiments; *p<0.01 by ANOVA. In FIG. 2E, IDO-preactivated Tregs were sorted and added to readout assays containing A1 T cells plus either CBA DCs or CBA B cells. One of three experiments; *p<0.01 by ANOVA.

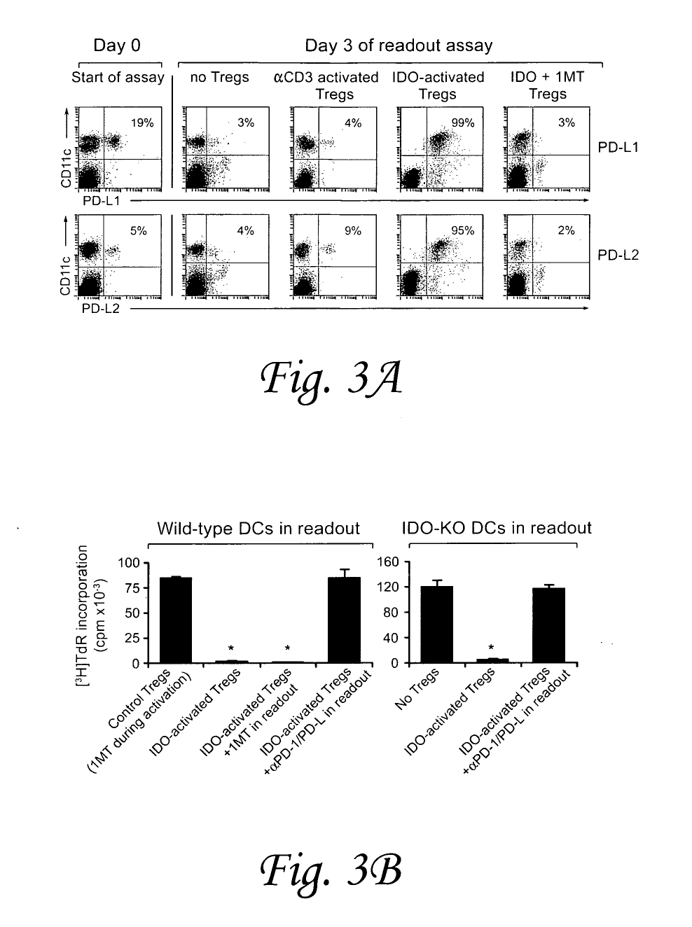

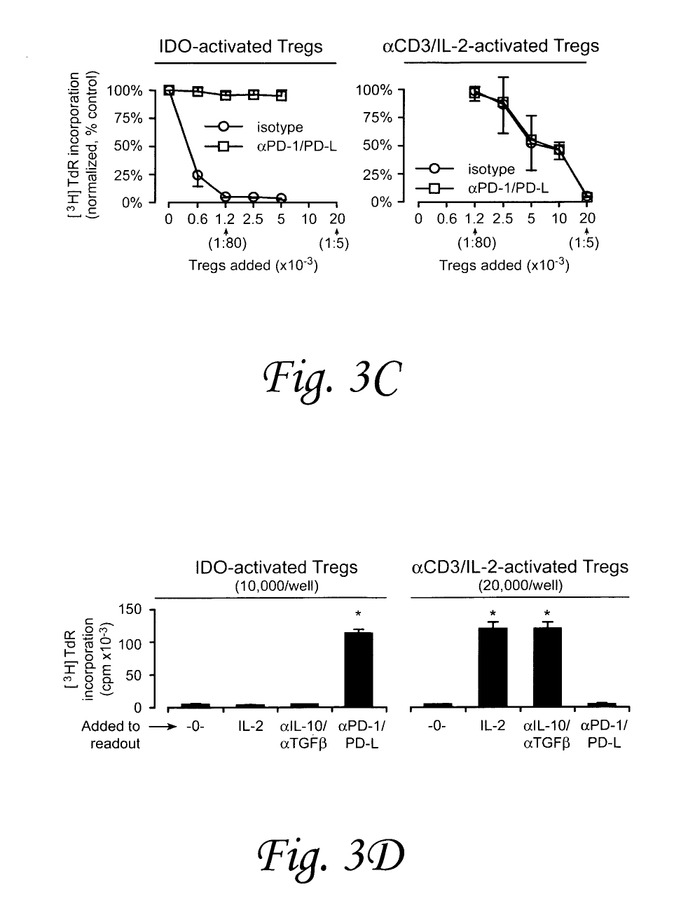

FIG. 3 shows suppression by IDO-activated Tregs requires the PD-1/PD-L pathway. In FIG. 3A, Tregs were preactivated with IDO⁺ pDCs as in FIG. 2, then 1×10⁴ sorted Tregs were added to readout assays (A1 T cells+CBA DCs). After 24 hours, cultures were harvested and stained for PD-L1 and PD-L2 relative to CD11c. FIG. 3A shows one of three experiments. IN FIG. 3B, IDO-activated Tregs (5000/well) were added to readout assays (A1 T cells plus either wild-type CBA DCs or IDO-KO DCs on the CBA background). Readout assays received either no additive, 1MT, or a cocktail of blocking antibodies against PD-1, PD-L1 and PD-L2 (50 ug/ml each). Control Tregs received 1MT during the pre-activation step. FIG. 3B shows one of three experiments; *p<0.01 by ANOVA. In FIG. 3C, Tregs were activated with IDO pDCs, or in identical cultures containing 1MT to block IDO and anti-CD3 plus IL-2 to activate the Tregs. After sorting, Tregs were added to readout assays (A1 T cells+CBA DCs), with or without PD-1/PD-L blocking antibodies as shown. Graphs show the mean±SD of ten independent experiments with IDO-activated Tregs, and three experiments with anti-CD3-activated Tregs, using TDLN pDCs from B78H1-GMCSF and B16-OVA tumors. In FIG. 3D, IDO-activated Tregs (1×10⁴/well) and anti-CD3/IL-2-activated Tregs (2×10⁴/well) were prepared as in the previous panel, and added to readout assays with or without recombinant IL-2, anti-IL-10 plus anti-TGF-β blocking antibodies (100 μg/ml each), or PD-1/PD-L blocking antibodies. Bars show SD for replicate wells in one of four similar experiments; *p<0.01 by ANOVA.

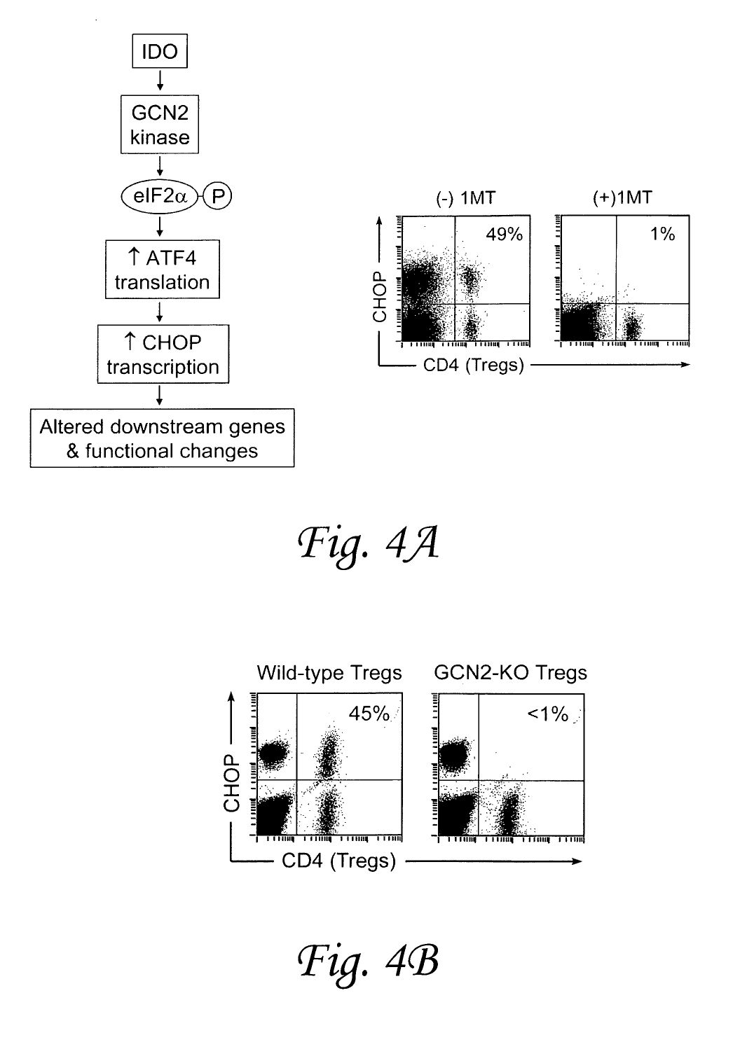

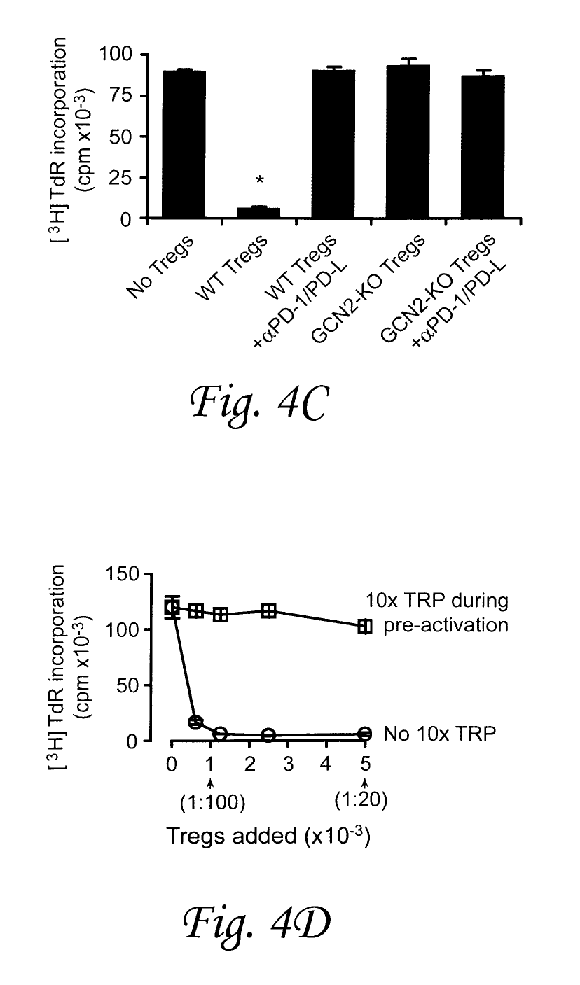

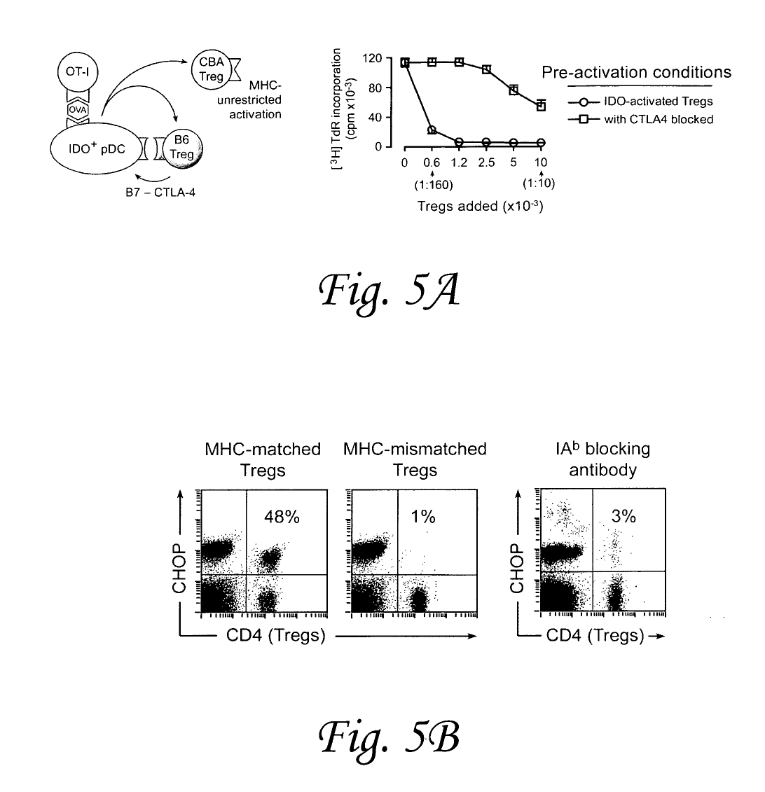

FIG. 4 shows IDO-induced activation requires GCN2-kinase in Tregs. In FIG. 4A, activation cultures were set up with Tregs, TDLN pDCs, OT-I cells and feeder cells, with or without 1MT. After two days, intracellular staining was performed for CHOP expression in Tregs (CD4⁺ cells). The percentages show the fraction of Tregs that were CHOP⁺. FIG. 4A is one of nine similar experiments. In FIG. 4B, as in the preceding panel, compares Tregs derived from wild-type mice versus GCN2-KO mice (each assay with OVA, without 1MT). One of three experiments. In FIG. 4C, Tregs from GCN2-KO mice or wild-type controls were pre-activated with IDO⁺ pDCs as in FIG. 2, re-sorted, and 5000 Treg added to readout assays (A1 T cells+CBA DCs), with and without PD-1/PD-L blocking antibodies. One of three similar experiments; *p<0.01 by ANOVA. In FIG. 4D, Tregs from wild-type mice were pre-activated with IDO⁺ pDCs, re-sorted, and tested in readout assays with and without added 10× tryptophan (250 μM). Bars show SD for replicate wells. One of three similar experiments is shown.

FIG. 5 shows MHC-dependent and independent steps in IDO-induced Treg activation. In FIG. 5A B6, Tregs were activated with IDO⁺ pDCs as in FIG. 2, with or without anti-CTLA4 blocking mAb (10 μg/ml) during the activation step. Activated Tregs were re-sorted and tested in readout assays (A1 T cells plus CBA DCs). Bars show SD for replicate wells in one of four similar experiments. In FIG. 5B, CHOP induction in Tregs is MHC-restricted. Cultures were set up as in FIG. 4A, and cells stained for CHOP after two days. The left-hand plot shows assays using Tregs that were MHC matched to the IDO+ pDCs (B6 background); the second plot shows assays with MHC mismatched (CBA) Tregs. The right plot shows cultures with MHC-matched B6 Tregs but with 100 μg/ml blocking antibody to Ia^{b}. Controls without blocking antibody, or with irrelevant antibody, were similar to the first plot. One of four experiments. In FIG. 5C, the left graph shows activation co-cultures were set up as in FIG. 2, using MHC mismatched (CBA) Tregs. After two days, CBA Tregs were re-sorted and added to readout assays (A1 T cells plus CBA DCs). In FIG. 5C, (right-hand graphs), identical assays, except that CBA Tregs were mixed with Thy1.1 congenic B6 Tregs (10,000 each) during the pre-activation co-cultures, then each Treg population was re-sorted and tested separately. Bars show SD for replicate wells in one of three similar experiments, using TDLN pDCs from B78H1-GMCSF and B16-OVA tumors.

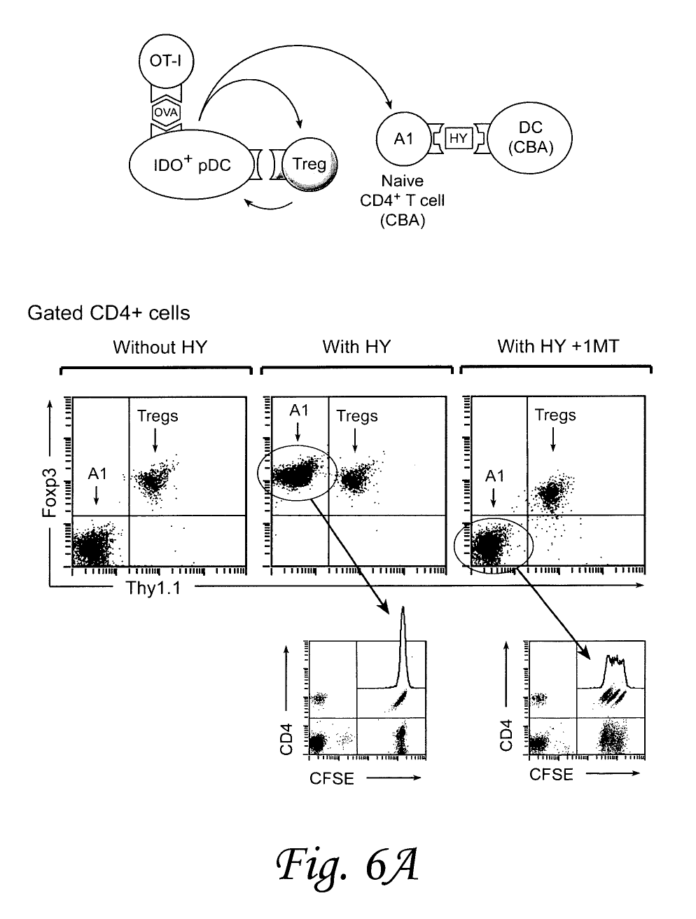

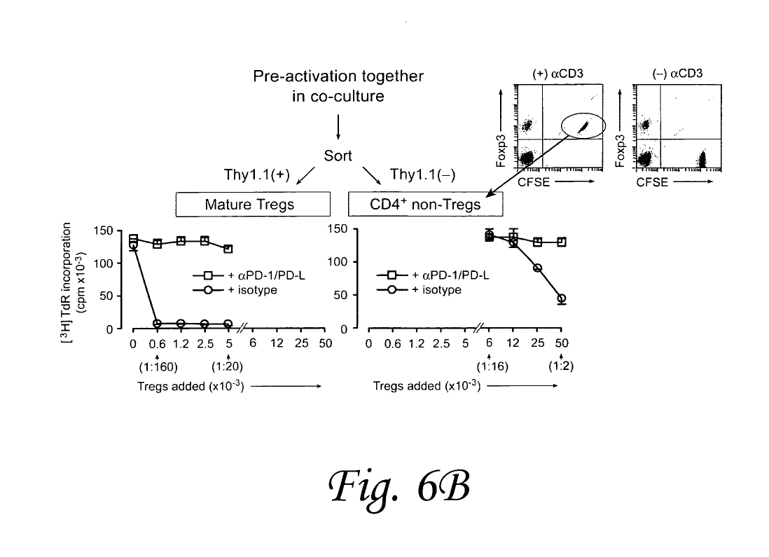

FIG. 6 shows direct activation of mature Tregs is more potent than de novo differentiation of new Tregs. In FIG. 6A, activation cocultures were set up as in FIG. 2, using Thy1.1-congenic B6 Tregs. To these were added CD4+CD25NEG (naive, non-regulatory) T cells from A1 mice plus CBA spleen DCs. Parallel groups received either no H-Y antigen for the A1 cells, H-Y, or H-Y+1MT. All cultures received OVA peptide for the OT-I cells. After two days, cocultures were stained for CD4, Foxp3, and Thy1.1. The inset dot-plots show similar cultures in which the A1 and OT-I cells were labeled with CFSE prior to addition, then analyzed for cell division at the end of the assay. CFSE histograms for the A1 cells (CD4⁺ CFSE⁺) are superimposed. One of four experiments. In FIG. 6B, assays were set up as in the previous panel, using Thy1.1 congenic Tregs plus nonregulatory CD4⁺CD25^{NEG} cells from wild-type B6 mice, activated with anti-CD3 mAb. Inset dot dotplots document upregulation of Foxp3 in this model, using CD4⁺CD25^{NEG} cells pre-labeled with CFSE. After two days the Treg and non-Treg populations were sorted separately based on Thy1.1 expression, and tested in readout assays (A1 T cells plus CBA DCs). One of three similar experiments; bars show SD.

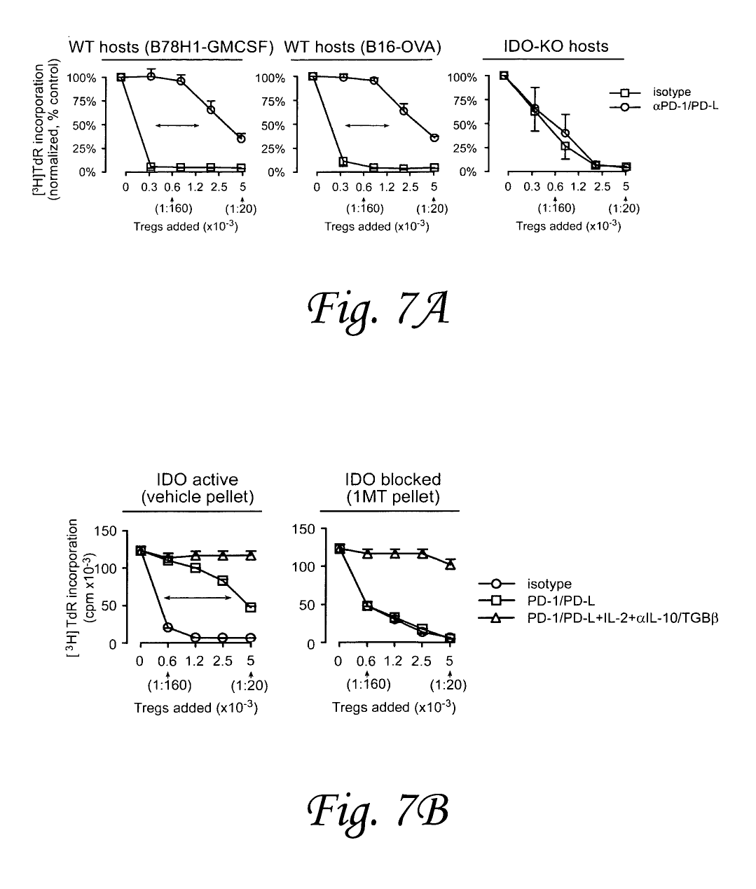

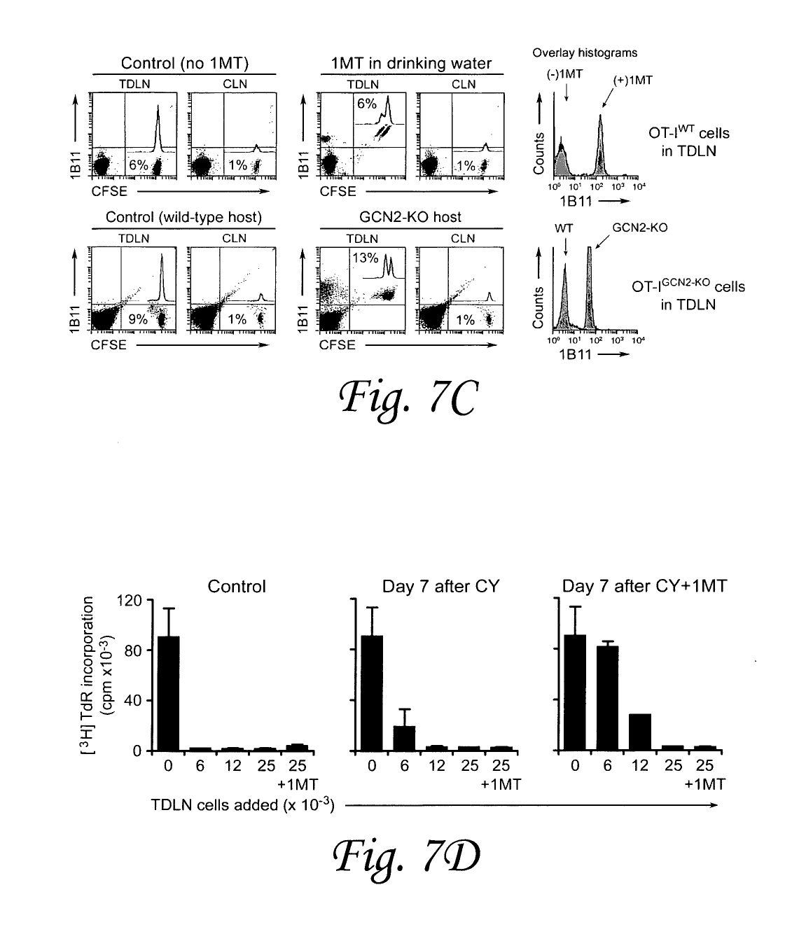

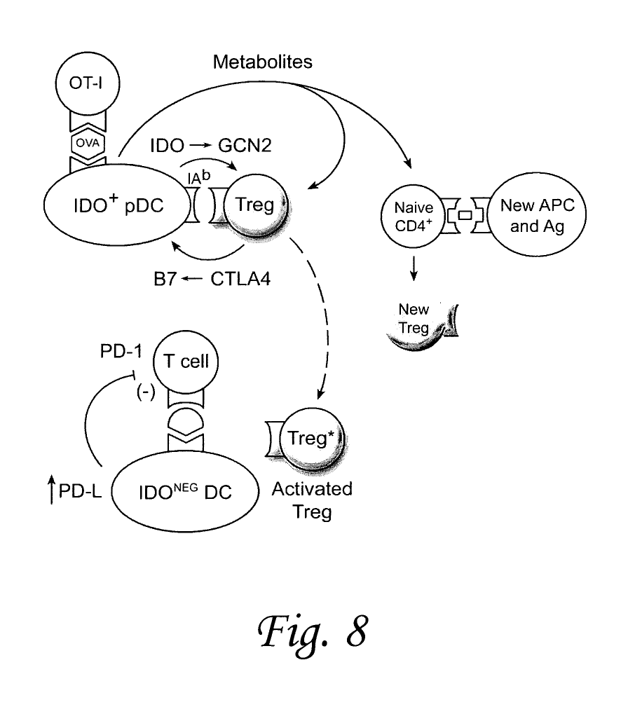

FIG. 7 shows IDO-activated Tregs in TDLNs. In FIG. 7A, tumors were grown in wild-type or IDO-KO hosts. Tregs from day seven TDLNs were sorted and added to readout assays (A1 T cells plus CBA DCs), with and without PD-1/PD-L blocking antibodies. Means of four pooled experiments with B78H1-GMCSF, four experiments with B16-OVA, and three experiments with IDO-KO hosts (two with B78H1-GMCSF and one with B16-OVA). In FIG. 7B, wild-type mice were treated throughout tumor growth with vehicle control (“IDO active”) or sustained-release 1MT (“IDO blocked”). Tregs from day seven tumors were tested in readout assays as above, with added isotype, PD-1/PD-L blocking antibodies, or a combination of anti-PD-1/PD-L plus IL-2 plus anti-IL-10/TGF-β antibodies. One of three experiments, using B78H1-GMCSF and B16-OVA. In FIG. 7C (upper panels), CFSE-labeled OT-I cells were injected into mice with B16-OVA tumors (day 7-8), with and without oral 1MT administration after transfer. After four days, TDLNs and contralateral LNs (CLN) were stained for the 1B11 activation marker. Percentages show the CFSE+ OT-I cells in total LN cells. Overlay histogram shows 1B11 on OT-I cells in TDLNs. Representative of four transfers each. In FIG. 7C (lower panels), similar experiments, using OT-IGCN2-KO cells transferred into WT or GCN2-KO hosts bearing B16-OVA tumors. One of three similar experiments. In FIG. 7D, B78H1-GMCSF tumors were treated on day 11 with vehicle (control), cyclophosphamide (CY, 150 mg/kg), or CY+1MT pellets. Seven days later, cells from TDLNs were harvested and added to readout assays (allospecific BM3 T cells plus B6 splenocytes, as described (Munn et al., 2004, J Clin. Invest; 114:280-290)). One control received 1MT added to the readout assay, as shown. One of three experiments is shown.

FIG. 8 is a proposed hypothetical model of IDO-induced Treg activation based on synthesis of results from the in vitro models. The interaction of resting Tregs with IDO⁺ pDCs results in activation of the Tregs through a combination of the GCN2 activation and tryptophan metabolites. Activated Tregs then suppress target T cells in an IDO-independent fashion, involving PD-ligand expression on the target DCs, and PD-1 expression (presumably on the target T cells). In addition, bystander CD4⁺ T cells responding to other antigens, if exposed to the conditions created by activating Tregs and IDO⁺ pDCs, are biased to differentiate into new Tregs.

FIG. 9 shows IDO expression by the CD19+ cells in the pDC fraction of TDLNs. IDO staining of cytocentrifuge preparations of sorted CD19⁺ pDCs (CD11c⁺B220⁺CD19⁺ cells) from TDLNs of B78H1-GMCSF tumors shows that this CD19⁺ subset of pDCs contains essentially all of the functional IDO-mediated suppressor activity in these TDLNs (see also, Munn et al., 2004, J Clin Invest; 114:280-290). Control cells (the non-plasmacytoid DC fraction of normal LNs) showed minimal IDO staining. Staining controls (neutralization of the primary antibody with an excess of the immunizing peptide) were all negative, as previously described (Munn et al., 2002, Science; 297:1867-1870). As shown in FIG. 9, FACS plots of gated B220⁺ cells from TDLNs show the CD11c⁺ CD19⁺ subset (the CD19⁺ pDCs sorted at left). Quantitatively, the B78H1-GMCSF tumors yielded about twice as many CD19⁺ pDCs as the B16-OVA tumors, as shown in Table 1, below.

TABLE 1

| | B78H1-GMCSF | B16-OVA |
|---|---|---|
| Total cells/TDLN (millions) | 4.2, 4.3, 4.0 | 3.6, 3.2, 3.4 |
| Absolute CD19⁺ pDCs/LN | 40, 49, 55 | 18, 15, 37 |
| (CD19⁺B220⁺CD11c⁺) (thousands) | | |
| mean ± SD CD19⁺ pDCs/LN | 48 ± 7 | 23 ± 11 |
| (thousands) | | |

The CD19⁺ subset of pDCs typically comprised 30-50% of total pDCs in TDLNs. Because the number of CD19⁺ pDCs was so small, their viability was improved if they were sorted as part of the total pDC fraction (CD11c⁺ B220⁺). Therefore, this was the preparation routinely used as the source of IDO-expressing cells for functional assays, as previously reported (Munn et al., 2004, J Clin Invest; 114:280-290 and Munn et al., 2005, Immunity; 22:633-642). Since the effects of IDO were dominant, it was immaterial whether other IDO^{NEG} pDCs were also present in the assays.

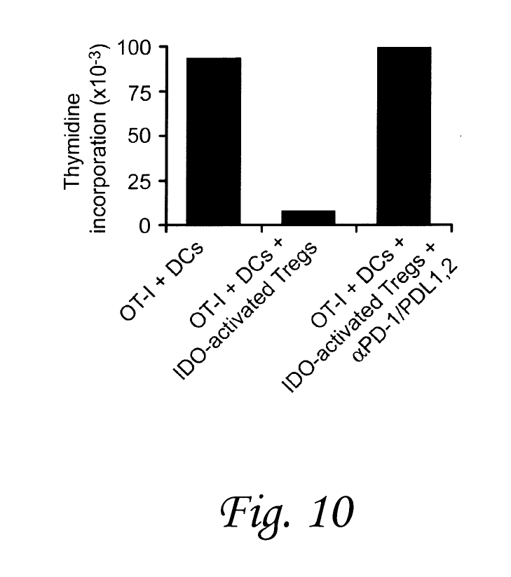

FIG. 10 shows that IDO-activated Tregs can suppress CD8+ T cells. Tregs were activated for two days in co-culture with TDLN pDCs plus OT-I cells plus OVA peptide (activation cultures, as described in FIG. 2). Activated Tregs were harvested, resorted, and added to readout assays comprising CD8⁺ OT-I T cells plus CD11c splenic DCs from B6 mice plus OVA peptide.

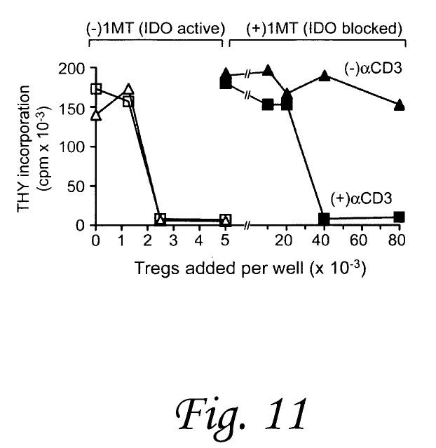

FIG. 11 shows that Tregs mediate suppression of bystander A1 cells in mixed co-cultures. A1 T cells and CBA DCs were added directly to the Treg pre-activation assay at the start of culture. Thus, the combined cultures comprised IDO⁺ pDCs, OT-I T cells, Tregs, A1 T cells, CBA splenic DCs, and feeder layer. This approach does not distinguish which population(s) of T cells were proliferating, nor was it designed to distinguish direct suppression (mediated by the IDO itself, e.g., via soluble tryptophan metabolites) from suppression mediated by the IDO activated Tregs. However, the mixed co-cultures addressed the specific question of whether IDO and activated Tregs could produce effective levels of suppression rapidly enough that they could suppress the bystander A1 cells before they could begin dividing. A titration of Tregs was added to the mixed co-culture assays described above, either with IDO active (no 1MT added), or with IDO blocked by 1MT, as shown. In each case, parallel titrations were performed with or without the addition of anti-CD3 mAb. The A1 and OT1 cells were already maximally activated by their respective cognate peptides, and anti-CD3 showed no further effect on these cells; the relevant effect of the anti-CD3 was thus to activate the Tregs. In the absence of Tregs there was substantial proliferation of T cells in co-cultures, despite the presence of IDO⁺ pDCs. However, the addition of fewer than 5000 Tregs was sufficient to suppress proliferation of all cells in culture. Suppression was not further enhanced by anti-CD3 mAb, indicating that Tregs were already maximally suppressive. In contrast, when IDO was blocked by 1MT, then even 10-fold more Tregs showed no spontaneous suppressor activity in the absence of anti-CD3 mAb. The addition of anti-CD3 mAb allowed Tregs to suppress even without active IDO, but suppression was an order of magnitude less effective than when IDO was active. Thus, the results of the mixed co-culture model were similar to those using the separate pre-activation and re-sorting step.

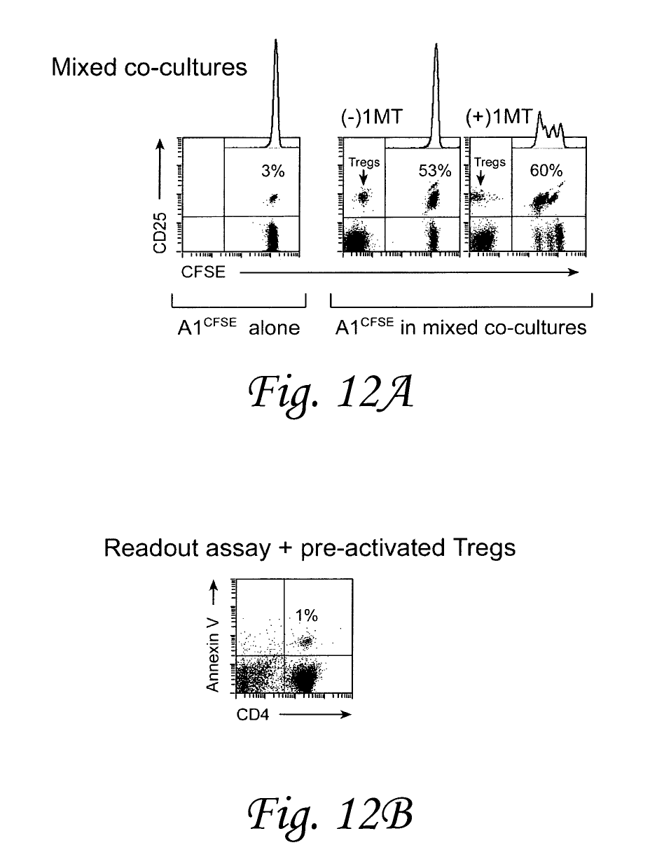

FIG. 12 shows suppressed A1 cells upregulate activation markers but do not divide. In FIG. 12A, mixed co-cultures were established as in FIG. 11, comprising Treg activation cultures (IDO⁺ pDCs, OT-I cells, Tregs, and feeder layer) plus the direct addition of CFSE-labeled CD4⁺ sorted A1 T cells plus CBA DCs plus HY peptide. After 2-3 days the mixed co-cultures were harvested and stained for CD25 versus CFSE. Percentages show the fraction of A1 cells that were CD25⁺. Similar results were also obtained with CD44 staining. Without activation, A1 CFSE cells were less than 5% CD25⁺. In co-cultures containing IDO-activated Tregs (without 1MT), the A1CFSE cells showed upregulation of activation markers (CD25 and CD44) on a proportion of cells, but were not able to divide. When IDO was blocked (plus 1MT) the A1CFSE cells upregulated activation markers and were able to divide. In FIG. 12B, IDO-activated Tregs were sorted as described in FIG. 2 and added to readout assays of A1 cells plus CBA DCs plus HY peptide. After three days, cells were harvested and stained for CD4 versus annexin V-PE. Minimal apoptosis of the suppressed A1 cells was observed.

FIG. 13 shows Tregs increase IDO enzymatic activity in a CTLA4-dependent fashion. TDLN pDCs (1×10⁴) and OT1 T cells (1×10⁵) were cultured for three days, with or without 1×10⁴ Tregs. Replicate wells received 10 μg/ml anti-CTLA4-blocking antibody (clone 9H10), and/or 1MT, as shown. After three days, the culture supernatants were analyzed by HPLC for the concentration of kynurenine (Munn et al., 1999, J Exp Med; 189:1363-1372). The absolute amount of kynurenine that accumulated in the supernatant was variable, since kynurenine is metabolized to other downstream products, but the relative amounts were informative. The arrows show that the addition of Tregs to the culture increased the production of kynurenine above the basal level produced by the IDO⁺ pDCs and OT-I alone; and that this Treg-induced increase was blocked by anti-CTLA4 mAb. The basal level of IDO, which was fully sufficient to inhibit the proliferation of the OT-I cells, was not blocked by anti-CTLA4 mAb (second bar). Thus, the effect of Tregs was to cause a “superinduction” of IDO, in a CTLA4-dependent fashion, which can be partially reversed by blockade with anti-CTLA4 antibody and totally reversed.

FIG. 14 shows IDO-induced Treg activation cannot be created when the medium contains insufficient tryptophan. To determine if the putative soluble factor responsible for Treg activation might be a metabolite of tryptophan, it was tested whether Treg activation would be prevented if the total available of tryptophan in the culture medium was made artificially low. Because each metabolite is made in a 1:1 stoichiometry, the level of metabolites produced is strictly limited by the initial concentration of tryptophan. Cultures were set up containing TDLN pDCs plus Tregs plus OT-I+feeder cells, with normal or low concentrations of tryptophan in the medium. FIG. 14 shows that conducting the pre-activation step in 2.5 μM tryptophan ( 1/10th the usual concentration) completely prevented the pre-activation of Tregs by IDO. This lower level of tryptophan was still ample to fully support proliferation of effector T cells (Munn et al., 2004, J Clin Invest; 114:280-290), so the reduced level of tryptophan itself was not toxic. Thus, the inability of IDO to activate Tregs under conditions of low tryptophan suggested that there might be an obligate role for tryptophan metabolites in IDO-induced Treg activation.

Tregs from TDLNs are highly activated. First, the activation status of Tregs from TDLNs was tested. B16 melanoma tumor cell lines were implanted in syngeneic C57BL/6 (B6) mice. Cell lines included B78H1-GMCSF (a subline of B16 transfected with GMCSF (Huang et al., 1994, Science; 264:961-965)), the noninfected B16F10 subline of B16, and B16-OVA (the B16F10 subline transfected with ovalbumin). Mice were studied on day 7-11 after tumor implantation. All TDLNs contained a population of cells that constitutively expressed IDO (FIG. 1A), which was not seen in non-tumor-draining (contralateral) LNs. As previously shown (Munn et al., 2004, J Clin Invest; 114:280-290), these IDO⁺ cells are a subset of DCs expressing plasmacytoid surface markers (CD11c⁺B220⁺) and coexpressing the marker CD19 (FIG. 9).

The IDO⁺ cells in TDLNs of all three tumor lines were similar. For most cell-sorting experiments B78H1-GMCSF tumors were used, as previously published (Munn et al., 2004, J Clin Invest; 114:280-290 and Munn et al., 2005, Immunity; 22:633-642), because these gave the highest yield of pDCs (see FIG. 9 and Table 1). However, pDCs from tumors without GMCSF gave similar functional results, and all key findings were confirmed with both types of tumors.

FIG. 1B shows analysis of Foxp3⁺ CD4⁺ Tregs in TDLNs. Both the TDLN and contralateral (non-draining) LNs contained a similar percentage of Tregs. However, when these Tregs were sorted by flow cytometry (CD4⁺CD25⁺ cells, >90% Foxp3⁺) and tested for functional suppressor activity, the Tregs from TDLNs were potently and spontaneously suppressive, whereas the Tregs from contralateral LNs showed no spontaneous suppressor activity (FIG. 1C). Tregs from TDLNs showed essentially complete suppression at a ratio of Tregs to readout T cells of <1:100, which was as potent as the most highly pre-activated Tregs achievable in vitro (McHugh et al., 2002, Immunity; 16:311-323 and Caramalho et al., 2003, J Exp Med; 197:403-411). In these experiments, the goal was to test whether the Tregs from TDLNs were constitutively activated in vivo, as opposed to becoming activated during the readout assay. Therefore, a readout system that was MHC-mismatched to the B6 Tregs (comprising TCR-transgenic A1 T cells and splenic DCs the CBA background) was selected. The use of an allogeneic readout assay minimized any possible activation of the Tregs by the APCs in the readout assay, and no additional mitogen or anti-CD3 crosslinking was added. In assays of this type, resting Tregs do not show suppression (Thornton and Shevach, 1998, J Exp Med; 188:287-296 and Nishikawa et al., 2005, J Exp Med; 201:681-686), whereas pre-activated Tregs are suppressive (Thornton and Shevach, 2000, J Immunol; 164:183-190). Thus, this assay allowed the degree of Treg pre-activation in vivo to be measured.

IDO⁺ DCs from TDLNs activate Tregs in vivo. To test the hypothesis that Treg activation in TDLNs might be related to the presence of IDO-expressing DCs, the DC population (CD11c⁺ cells) from TDLNs was isolated and transferred to new, non-tumor-bearing hosts. The phenotype of this mixed DC population has been previously described (Munn et al., 2004, J Clin Invest; 114:280-290), and typically contained 30-50% IDO-expressing CD19⁺ pDCs.) To test for the contribution of IDO, recipient mice were treated with the IDO-inhibitor drug 1-methyl-D-tryptophan (1MT) (Hou et al., 2007, Cancer Res; 67:792-801) beginning at the time of DC adoptive transfer, or with vehicle control. Because IDO does not become fully active until IDO⁺ DCs present antigen to responding T cells (Munn et al., 2004, J Immunol; 172:4100-4110), the DCs were pulsed with a peptide from chicken ovalbumin (OVA) and hosts pre-loaded with OVA-specific T cells (OT-I). Four days after DC transfer, host Tregs were sorted from LNs draining the site of DC injection and tested for suppressor activity. FIG. 1D shows that Tregs exposed to DC from TDLNs became potently activated, and this activation was blocked when recipient mice were treated with 1MT. Thus, the DC fraction from TDLNs, by itself, was sufficient to activate resting Tregs in new hosts, in an IDO-dependent fashion.

IDO⁺ pDCs from TDLNs activate resting Tregs in vitro. To study the mechanism of IDO-induced Treg activation, the two step model shown in FIG. 2 was used. Resting Tregs, from spleens of mice without tumors, were co-cultured with IDO⁺ DCs from TDLNs, then re-sorted and transferred to readout assays (A1 T cells+CBA DCs) to measure suppression. The IDO⁺ DCs were enriched from TDLNs by sorting for the plasmacytoid DCs (pDC) fraction, which have been previously shown to include essentially all of the IDO⁺ DCs in TDLNs in this system (see FIG. 9). Similar to human DCs (Munn et al., 2004, J Immunol; 172:4100-4110), DCs from TDLNs required triggering signals from T cells at the time of antigen presentation in order to express functional IDO enzymatic activity; this was supplied by allowing the pDCs to present OVA peptide to OT-I T cells. Co-cultures also contained a feeder layer of T-depleted spleen cells as described in the Methods section. After 30-48 hours, co-cultures were harvested and the Tregs recovered by sorting for CD4⁺ cells. Since the Tregs were the only CD4⁺ cells in the co-cultures, they could be unambiguously recovered.

FIG. 2A shows that resting Tregs exposed to IDO⁺ pDCs mediated potent suppression of T cell proliferation in readout assays. In contrast, if IDO was blocked by adding 1MT to the activation cultures, then the re-sorted Tregs showed no suppressor activity, similar to the resting Tregs from contralateral LNs. In the remainder of this example, Tregs activated by IDO⁺ pDCs from TDLNs are referred to as “IDO-activated Tregs,” since IDO was necessary for activation, recognizing that additional signals besides IDO may also be supplied by these TDLN pDCs. IDO-activated Tregs were able to suppress CD8⁺ T cells as well as CD4⁺ T cells in the readout assays (see FIG. 10). Pre-activation occurred within 30 hours, and was sufficiently rapid that IDO-activated Tregs were able to suppress all proliferation of readout cells, even if the A1 cells and CBA DCs were added directly to the Treg pre-activation assay at the beginning of cultures and allowed to activate in parallel (shown in FIG. 11). The A1 T cells in the readout assays were suppressed by activated Tregs but they were not killed, as shown by the fact that recovery of CD4⁺ cells at the end of three days was 95±8% of the expected cell number compared to controls (n=5 experiments), and Annexin V staining at the end of the three day assay was negative (see FIG. 12).

FIG. 2B shows a quantitative comparison of IDO-activated Tregs versus the same Tregs activated using the widely used approach of anti-CD3 crosslinking (Thornton et al., 2004, Eur J Immunol; 34:366-376). Both activation cultures contained identical cell populations, but the anti-CD3 cultures received 1MT to block IDO plus anti-CD3 and recombinant IL-2 to activate the Tregs. After activation and sorting, the IDO-activated Tregs mediated potent suppression, while the anti-CD3-activated Tregs were activated but quantitatively less suppressive (50% inhibition at a Treg:target cell ratio of 1:10, which is consistent with the findings of others using the anti-CD3 system (Caramalho et al., 2003, J Exp. Med; 197:403-41 land Thornton et al., 2004, Eur J Immunol; 34:366-376)).

FIG. 2C shows similar co-cultures, but with the TDLN pDCs replaced by various DCs that do not express IDO. The first graph (positive control) shows Tregs co-cultured with TDLN pDCs (IDO⁺). The middle left graph shows co-cultures using the non-plasmacytoid (CD11c⁺B220^{NEG}) DCs from the same TDLNs. The middle right graph shows co-cultures using pDCs from LNs of mice without tumors. The right graph shows cultures containing pDCs isolated from TDLNs of tumors grown in IDO-knockout (IDO-KO) hosts. Only the plasmacytoid DC fraction, derived from TDLNs, and with an intact host IDO gene, was able to activate Tregs. These data, combined with the complete abrogation of activation by 1MT (FIG. 2A), support a mechanistic role for IDO in mediating Treg activation.

Next, whether IDO-activated Tregs required physical contact with readout T cells in order to cause suppression was tested (FIG. 2D). IDO-activated Tregs were added to the lower well of transwell chambers, and readout cells (A1 T cells plus CBA DCs) were placed in both the lower chamber (in contact with the Tregs) and in the upper chamber (separated by a microporous membrane). Separate thymidine incorporation assays were performed on each chamber, and showed that the IDO-activated Tregs suppressed those T cells with which they were in contact, but had no effect on T cells separated across the membrane.

Suppression by IDO-activated Tregs requires the PD-1/PD-L pathway. Certain forms of T cell suppression by Tregs can be mediated indirectly via an effect on the target APCs (Bluestone and Tang, 2005, Curr Opin Immunol; 17:638-642). Therefore whether suppression by IDO-activated Tregs required the participation of the DCs was tested in the readout assays. FIG. 2E shows that IDO-activated Tregs were unable to suppress proliferation of A1 T cells when B cells were substituted instead of DCs as APCs in the readout assay. Similar loss of suppression was seen when anti-CD3/CD28 coated beads were substituted for the DCs. This suggested that the suppressive effect of IDO-activated Tregs might be mediated indirectly, via an effect on the target DCs.

One mechanism by which DCs may suppress T cells is via the inhibitory programmed cell death 1 (PD-1)/programmed cell death ligand PD-ligand (PD-L) pathway (Probst et al., 2005, Nat Immunol; 6:280-286 and Curiel et al., 2003, Nat Med; 9:562-567). While this pathway has not previously been described as a mediator of Treg suppression, related B7 family members have been linked to Treg-induced suppression (Kryczek et al., 2006, J Immunol; 177:40-44). FIG. 3A shows that IDO-activated Tregs caused upregulation of both PD-L1 and PD-L2 on the DCs (CD11c⁺ cells) in readout assays. In contrast, PD-ligand expression by DCs was low in readout assays without Tregs, or in readout assays receiving Tregs from pre-activation cultures in which IDO was blocked with 1MT (FIG. 3A). Even readout assays receiving Tregs that had been activated with anti-CD3 plus IL-2 did not show upregulation of PD-ligands on DCs (FIG. 3A). Thus, the upregulation of PD-ligands on DCs appeared associated specifically with the form of Treg activation created by IDO.

Therefore, whether blocking the PD-1/PD-L pathway in the readout assay would prevent suppression by IDO-activated Tregs was studied. To ensure that the pathway was fully blocked, a cocktail of antibodies against PD-1, PD-L1 and PD-L2 was added to the readout assays. Blocking the PD-1/PD-L pathway completely abrogated the ability of IDO-activated Tregs to suppress T cell proliferation (FIG. 3B). In contrast, adding 1MT to the readout assay, or using DCs from IDO-KO mice, had no effect on T cell suppression. Thus, while IDO was strictly required to activate the Tregs initially (FIG. 2A), suppression of target cells by IDO-activated Tregs was independent of IDO, and was dependent on the PD-1/PD-L pathway.

FIG. 3C compares the role of the PD-1/PD-L pathway in suppression by IDO-activated Tregs versus Tregs pre-activated by anti-CD3 plus IL-2. Suppression by IDO-activated Tregs was completely prevented by blocking PD-1/PD-L in the readout assay, whereas suppression by anti-CD3-activated Tregs was unaffected by PD-1/PD-L blockade. In contrast, FIG. 3D shows that suppression by anti-CD3-activated Tregs was fully reversed by adding recombinant IL-2 to the readout assay, or by blocking IL-10 and TGF-β, while these manipulations had no effect on suppression by IDO-activated Tregs. Thus, the mechanisms of suppression by IDO-activated Tregs and anti-CD3-activated Tregs were distinct, and could be unambiguously distinguished based on sensitivity to PD-1/PD-L blockade, exogenous IL-2, and IL-10/TGF-β blockade.

GCN2-kinase is required for Treg activation. Next, whether Tregs responded to IDO via the GCN2-kinase pathway was tested. GCN2 kinase is activated by reduced levels of amino acids, as might occur when IDO depletes tryptophan (Harding et al., 2003, Mol Cell; 11:619-633). It has been previously shown that IDO activates GCN2 kinase in CD8⁺ effector T cells, leading to cell-cycle arrest and anergy in these cells (Munn et al., 2005, Immunity; 22:633-642). As diagrammed in FIG. 4, activation of GCN2 can be detected by measuring the downstream marker gene CHOP/gadd153 (Munn et al., 2005, Immunity; 22:633-642). Treg activation cultures were set up as in FIG. 2, and CHOP expression measured by intracellular staining after two days.

FIG. 4A shows that CHOP was upregulated when IDO was active and was expressed in both OT-I cells (visible as the CD4^{NEG} population) and Tregs (CD4⁺). In these studies, approximately half of the Tregs upregulated CHOP, which could reflect an intrinsic heterogeneity in the CD25⁺ Treg population. Blocking IDO with 1MT abrogated CHOP expression in OT-I cells as expected, and also prevented CHOP induction in Tregs, showing that both events were IDO-dependent (FIG. 4A). FIG. 4B shows that Tregs derived from mice lacking functional GCN2 (GCN2-KO mice) showed no IDO-induced upregulation of CHOP. Consistent with this, GCN2-KO Tregs were unable to undergo functional activation by IDO (FIG. 4C). GCN2-KO Tregs were still able to undergo anti-CD3-induced activation, so they were not globally deficient in suppressor activity. Finally, FIG. 4D shows that IDO-induced activation of wild-type Tregs was blocked by adding excess tryptophan to the pre-activation cultures. Taken together, these data were thus consistent with the hypothesis that a tryptophan-withdrawal stress, imposed by IDO and sensed via the GCN2-kinase pathway, was required for Treg activation by IDO⁺ pDCs.

CTLA4 blockade prevents Treg activation in co-cultures. Tregs themselves have been reported to upregulate IDO expression in DCs (Fallarino et al., 2003, Nat Immunol; 4:1206-1212). This occurs via binding of cell-surface CTLA4 on Tregs to B7.1/B7.2 molecules on DCs, resulting in B7-mediated induction of IDO (Mellor et al., 2003, J Immunol. 171:1652-1655 and Grohmann et al., 2002, Nat. Immunol; 3:1097-1101). Consistent with this, this example found that the addition of Tregs to co-cultures of TDLN pDCs plus OT-I T cells significantly increased IDO enzymatic activity (measured as production of kynurenine, the first major metabolite of tryptophan produced by IDO), and that this Treg-induced enhancement was prevented by blocking CTLA4 in co-cultures (FIG. 13). Likewise, blocking CTLA4 significantly inhibited IDO-induced functional activation of Tregs in co-cultures (FIG. 5A). Thus, IDO caused activation of Tregs, but a reciprocal interaction with the Tregs appeared necessary for full induction of IDO.

Distinct MHC-restricted and MHC-unrestricted components of activation. Next, whether interaction with MHC molecules on the pDCs was required for Treg activation was tested. FIG. 5B shows that induction of CHOP expression in Tregs was strictly dependent on interaction with the MHC molecules expressed on the IDO⁺ pDCs. CHOP was not induced if the Tregs and pDCs were mismatched at MHC class II (FIG. 5B), or if interaction with MHC was blocked by antibody against IA^{b} (the MHC-II allele expressed by B6 mice). Consistent with this, FIG. 5C shows that MHC mismatched CBA Tregs did not become activated during co-culture with IDO⁺ B6 pDCs. However, if the CBA Tregs were mixed with MHC-matched (B6Thy1.1) Tregs, then both populations became activated, and both mediated suppression via the characteristic IDO-induced PD-1/PD-L-dependent mechanism (FIG. 5C). This suggested that an MHC-restricted interaction between the pDCs and Tregs was required in order to trigger the effects of IDO (perhaps as part of the same CTLA4-dependent activation step shown above), but once IDO was triggered it could then affect other Tregs in the cultures, in an MHC-unrestricted fashion.

One potential mechanism to explain this MHC-unrestricted effect of IDO might be secretion of soluble metabolites of tryptophan (Fallarino et al., 2006, J Immunol; 176:6752-6761). FIG. 14 presents indirect evidence consistent with this possibility. However, the effects of IDO⁺ pDCs, including the induction of PD-1/PD-L-mediated suppressor activity, using purified tryptophan metabolites alone, have not been directly reproduced. Thus, while FIG. 14 suggests that tryptophan metabolites are important participants in IDO-induced Treg activation, their specific role remains to be determined.

IDO preferentially activates pre-existing Tregs. It has been previously shown that IDO can promote de novo differentiation of Foxp3⁺ Tregs from naive CD4⁺ T cells in vitro (Fallarino et al., 2006, J Immunol; 176:6752-6761). Therefore, whether IDO would induce naive CD4+ cells to differentiate into Foxp3⁺ cells in this system was studied. Cocultures were set up as shown for FIG. 6A, comprising IDO⁺ pDCs, OT-I, feeder cells, mature Tregs (Thy1.1 congenic), plus a population of CD4⁺CD25^{NEG} T cells (naive male-specific A1 T cells isolated from female mice). CBA splenic DCs were also added to serve as APCs for the A1 cells. After two days, co-cultures were harvested and stained for intracellular Foxp3.

FIG. 6A shows analysis of the CD4⁺ population from such an experiment. In the absence of their cognate H-Y peptide, none of the A1 cells expressed Foxp3 at the end of culture. In the presence of H-Y peptide, there was upregulation of Foxp3 in up to 95% of A1 cells, depending on the experiment. Upregulation of Foxp3 was prevented when IDO activity was blocked by 1MT. The inset dotplots show similar assays in which the A1 and OT-I T cells were labeled with CFSE, demonstrating that the A1 cells remained in a non-divided state when IDO was active, but divided when IDO was blocked by 1MT. Further studies demonstrated that the IDO-arrested A1 cells upregulated CD25 and CD44 in response to antigen (thus showing evidence of attempted activation), even though they could not divide. Thus, it was the combination of antigenic stimulation by H-Y peptide, plus forced cell-cycle arrest by IDO, that led to upregulation of Foxp3 in the A1 cells.

To confirm that upregulation of Foxp3 was not a peculiarity of the TCR-transgenic A1 system, similar experiments were performed using non-transgenic (polyclonal) CD4⁺CD25^{NEG} cells from wild-type B6 mice activated with anti-CD3 crosslinking, similar to previous studies Fallarino et al., 2006, J Immunol; 176:6752-6761). Identical upregulation of Foxp3 was observed in this system (FIG. 6B, left dot plot, in which the naive CD4⁺ cells were identified by CFSE staining). To ask whether the de novo Foxp3-expressing cells acquired functional activity, the cells were re-sorted and tested for suppressor activity. FIG. 6B shows that the mature Tregs from these cocultures became potently activated for suppression, whereas the naive CD4⁺ population acquired only a small amount of suppressor activity (100-fold less than the mature Tregs on a per-cell basis). Thus, within the length of time that the activation assays were performed, newly-differentiated Foxp3⁺ cells acquired little functional activity in response to IDO, whereas the mature, pre-existing Tregs became rapidly and potently activated.

IDO-induced Treg activation in TDLNs. In all the preceding studies, resting Tregs were activated by IDO in vitro. Next, it was tested whether Tregs isolated directly from TDLNs showed evidence of constitutive activation by IDO in vivo. Based on FIG. 3D, findings consistent with IDO-induced Treg activation were defined as spontaneous ex vivo suppression that was dependent on the novel PD-1/PD-L pathway and resistant to IL2 and 1L-10/TGF-β blockade. The “conventional” component of Treg activity was defined as suppression that was reversed by IL2 and IL-10/TGF-β blockade and was indifferent to PD-1/PD-L.

Tregs were sorted from TDLNs and added directly to readout assays (A1 T cells plus CBA DCs). FIG. 7A (left and middle panels) shows that the majority of suppression by TDLNs Tregs was prevented by PD-1/PD-L blockade. A small amount of residual PD-1/PD-L-independent activity remained at the higher Treg:effector ratios, consistent with a mixture of both conventional and IDO-induced forms of suppression. Based on the shift in IC50, 75-90% of suppression by TDLN Tregs appeared mediated via the PD-1/PD-L pathway. In contrast, when tumors were grown in IDO-KO mice, the Tregs in TDLNs completely lacked the PD-1/PD-L-mediated component of suppression (FIG. 7A, right panel).

Similar results were obtained when the IDO pathway was pharmacologically inhibited by administering 1MT during the period of tumor growth (FIG. 7B). To test whether the residual, non-PD-1/PD-L-mediated component of suppression represented “conventional” Treg activity, it was tested whether exogenous IL-2 plus anti-IL-10/TGF-β blockade would reverse this residual component of Treg suppression. FIG. 7B shows that this manipulation completely reversed all of the remaining components of suppression. In the case of tumors grown in the absence of IDO (mice receiving 1MT, FIG. 7B), the conventional (IL-2/anti-IL-10/TGF-β-reversible) form of suppression accounted for all of the Treg activity in TDLNs, and none was of the PD-1/PD-L-dependent type.

Inhibition of T cell responses in TDLNs in vivo. Next, this example tested whether in vivo T cell responses were suppressed in TDLNs. OT-1 T cells were labeled with CFSE tracking dye and injected intravenously into mice bearing established B16-OVA tumors (which express the cognate antigen for OT-I). FIG. 7C (upper panels) shows that OT-I cells preferentially accumulated in the TDLN four days after injection (6% of cells in TDLN versus 1% in contralateral LN), but they showed no cell division and no evidence of activation (assessed as upregulation of the 1B11 T cell activation marker (Harrington et al., 2000, J Exp Med; 191:1241-1246)). To ask whether this lack of response was related to IDO expression, recipient mice were treated with 1MT. When IDO was blocked with 1MT, OT-I in TDLNs became able to uniformly upregulate the activation marker 1B11 (overlay histograms), thus showing evidence of attempted activation, although they were still not able to undergo extensive cell division.

The use of 1MT could not distinguish between direct suppression of OT-I cells by IDO itself, versus an IDO-induced activation of host suppressor cells (e.g., IDO-activated Tregs). To eliminate any direct effect of IDO on the OT-I cells, OT-I mice bred onto the GCN2-KO background (OT-IGCN2-KO) were used. This renders the OT-IGCN2-KO T cells refractory to the direct suppressive effects of IDO, as we have previously shown (Munn et al., 2005, Immunity; 22:633-642), but they remain fully susceptible to suppression by Tregs, since this is independent of IDO (see FIG. 3B). Since IDO could not directly suppress the OT-IGCN2-KO cells, any effect of IDO would have to be exerted via IDO responsive host suppressor cells. This host response to IDO could be controlled by making the recipient mice either GCN2-sufficient or GCN2-KO, as shown in FIG. 7C (lower panels). When recipient mice were GCN2-sufficient, transferred OT-IGCN2-KO T cells remained fully suppressed in TDLNs; however, the same OT-IGCN2-KO cells became able to activate if the recipient mice were GCN2-KO (and hence unable to respond to IDO). These data thus supported the preceding data using 1MT, and, taken together, were consistent with a population of IDO-responsive suppressor cells in TDLNs, derived from the host, and activated by IDO in a GCN2-dependent fashion.

Chemotherapy plus 1MT in vivo depletes suppressor activity in TDLNs. From a clinical standpoint, constitutive activation of Tregs in TDLNs could represent a formidable barrier to immunotherapy. Certain chemotherapeutic drugs such as cyclophosphamide have been reported to partially reduce the number and/or function of Tregs (Ghiringhelli et al., 2004, Eur J Immunol; 34:336-344 and Lutsiak et al., 2005, Blood; 105:2862-2868). It has previously been shown that 1MT displays immune-mediated synergistic anti-tumor effects when combined with chemotherapy (Muller et al., 2005, Nat Med; 11:312-319, U.S. Patent Application Serial No. 20040234623, and Hou et al., 2007, Cancer Res; 67:792-801). Therefore, whether cyclophosphamide combined with 1MT could reduce the suppressor activity found in TDLNs was tested. For these experiments, the suppressor activity in total, unfractionated TDLN cells was measured, as previously described (Munn et al., 2004, J Clin Invest; 114:280-290), because the cell number in TDLNs after chemotherapy was too small to permit sorting of individual cell populations. FIG. 7D shows that in untreated control mice, the TDLN cells were intensely suppressive for the readout assays (proliferation of CD8⁺ T cells). This suppression was not affected by the addition of 1MT to the readout assays (final bar in each graph), consistent with a significant component of suppression by activated Tregs. Treatment with cyclophosphamide alone reduced the suppressive activity only slightly. However, administration of cyclophosphamide followed by 1MT significantly reduced the suppressor activity by TDLN cells (FIG. 7D). This suggested that the potently suppressive milieu in TDLNs could be partially alleviated by the combination of chemotherapy plus 1MT.

### Discussion

This example demonstrates for the first time a mechanistic link between IDO, functional activation of Tregs, and the PD-1/PD-ligand pathway. Each of these mechanisms is known to be independently important in tumor immunology, and strategies targeting each mechanism are currently in clinical trials or in active preclinical development. This example now shows that these three powerful regulatory mechanisms are tightly linked at the level of Treg activation in the TDLN. This linked pathway constitutes a major contributor to the intensely immunosuppressive milieu present in TDLNs. Since this suppressive milieu drives T cell anergy and unresponsiveness to tumor antigens presented in the TDLNs (Munn et al., 2006, Immunol Rev; 213:146-158), identification of molecular mechanisms contributing to this suppression represents an important goal in cancer immunotherapy.

The findings of this example support the model shown in FIG. 8 in which IDO-induced Treg activation proceeds via a self-amplifying loop. When IDO pDCs present antigen to effector T cells in the presence of mature, resting Tregs, this initiates a GCN2-dependent activation of the Tregs by IDO. In other cells, GCN2 is known to activate a downstream stress-response pathway, resulting in a coordinated program of changes in gene expression (Harding et al., 2003, Mol Cell; 11:619-633 and Wek et al., 2006, Biochem Soc Trans; 34:7-11). In the case of CD8⁺ effector T cells, it has been previously shown that activation of the GCN2 pathway leads to cell cycle arrest and anergy (Munn et al., 2005, Immunity; 22:633-642). In the case of Tregs, this example now shows that GCN2 signaling in critical for allowing IDO-induced functional activation. Based on FIGS. 5A and 13, it can be concluded that the activating Tregs reciprocally induce high levels of IDO in pDCs, via CTLA4-B7 interaction (Fallarino et al., 2003, Nat Immunol; 4:1206-1212), leading to increased production of tryptophan metabolites. These metabolites then complete the full activation of the Tregs (as suggested by FIGS. 5C and 14), resulting in emergence of the novel, highly potent, PD-1/PD-L-dependent form of suppression. This model is consistent with the data presented in this example, and the two key central features of the model are clear and well supported: direct IDO induced activation of mature Tregs, and PD-1/PD-L-dependent suppression by IDO-activated Tregs.

A role for PD-1/PD-L as a downstream suppressor mechanism for Tregs has not been previously described. However, induction of another suppressive B7 family member, B7-H4, on APCs by Tregs has been recently shown (Kryczek et al., 2006, J Immunol; 177:40-44). In this system, the PD-1/PD-L mechanism of suppression was only found with the IDO-induced form of Treg activation, and was not seen with the widely studied anti-CD3-induced form of Treg activation. The PD-1/PD-L pathway has been the focus of considerable interest because it has been found to mediate clonal exhaustion and T cell anergy in HIV and other chronic viral infections (Sharpe et al., 2007, Nat Immunol; 8:239-245), as well as tolerance to self antigens and immune suppression in cancer (Okazaki and Honjo, 2006, Trends Immunol; 27:195-201).

This example provides a novel mechanistic link between the PD-1/PD-L system, Tregs and IDO. Tregs isolated from TDLNs in vivo were constitutively activated, displaying spontaneous suppressor activity that was as potent as the highest levels reported for Tregs extensively activated in vitro (McHugh et al., 2002, Immunity; 16:311-323 and Caramalho et al., 2003, J Exp Med; 197:403-411). The majority of this constitutive Treg activity in TDLNs was mediated via the novel IDO-induced, PD-1/PD-L-dependent mechanism. This example demonstrates the existence of two distinct, clearly distinguishable forms of Treg activity: the “conventional” form elicited by anti-CD3 crosslinking, in which suppression was dependent on IL10/TGF-β, was reversed by IL-2, and was unaffected by PD-1/PD-L blockade; and the novel IDO-induced form, which was not dependent on IL10/TGF-β, was not reversed by IL-2, and was strictly dependent on the PD-1/PD-L pathway. Under IDO-sufficient conditions, 75-90% of the constitutive Treg activity in TDLNs was due to the IDO-induced form of Treg activity. This IDO-induced component was completely lost when tumors were grown in IDO-KO mice, or in mice treated with an IDO-inhibitor drug during tumor growth. Under these chronically IDO-deficient conditions, tumors showed a compensatory increase in the form of Treg activity that was not dependent on IDO, consistent with emergence of tumor escape variants (Zitvogel et al., 2006, Nat Rev Immunol; 6:715-727). However, while tumors were thus able to compensate for artificial genetic or pharmacologic ablation of IDO, from a clinical standpoint, human patients would normally be IDO-sufficient. Thus, a key observation in this example was that 75-90% of the naturally-occurring Treg activity in TDLNs was of the IDO-induced, PD-1/PD-L-dependent form.

In vitro, IDO activity also promoted de novo upregulation of Foxp3 expression in naïve CD4⁺ T cells. This finding is not novel, since the pathway has already been described (Fallarino et al., 2006, J Immunol; 176:6752-6761). In the present system, the mature, pre-existing Tregs activated by IDO were 100-fold more potent on a per-cell basis than the newly-differentiated Foxp3⁺ cells. In human T cells, it is known that Foxp3 upregulation does not necessarily connote stable commitment to Treg differentiation (Wang et al., 2007, Eur J Immunol; 37:129-138 and Gavin et al., 2006, Proc Natl Acad Sci USA; 103:6659-6664), so it is possible that not all of the newly-derived Foxp3⁺ cells would go on to become Tregs. Nevertheless, it is relevant to note that IDO is potentially linked to the Treg lineage at two points: the rapid and potent activation of mature Tregs described herein, and the potential for de novo differentiation of new Tregs as well.

IDO-induced Treg activation was almost entirely prevented by blockade of CTLA4. CTLA4 has multiple regulatory roles in the immune system, most of which are intrinsic to the CTLA4⁺ T cells themselves; however, it is also known that CTLA4 can induce IDO expression in DCs, via back-signaling through B7 molecules (Fallarino et al., 2003, Nat Immunol; 4:1206-1212). It is likely that CTLA4 on Tregs delivers a signal to IDO⁺ pDCs that enhances their normal level of IDO enzymatic activity, and thus increases the production of immunoregulatory metabolites. Interpretation of such studies is complex, because it is difficult to separate cell-autonomous effects of the antibody on Treg function versus its effects on IDO, so further studies are required. However, from a therapeutic standpoint, anti-CTLA4 antibodies are in late-stage clinical trials (Peggs et al., 2006, Curr Opin Immunol; 18:206-213), so it is of interest to note that CTLA4 blockade also interrupts the novel IDO/Treg/PD-ligand pathway.

The human counterpart of the IDO pDCs in mouse TDLNs is not yet established, and human and mouse DC subsets do not always correspond. However, a prominent population of IDO-expressing cells is observed in many human TDLNs (Munn et al., 2002, Science; 297:1867-1870), displaying a characteristic plasmacytoid morphology (Lee et al., 2003, Lab Invest; 83:1457-1466). Recently, human plasmacytoid DCs (CD123⁺ BDCA2⁺) have been shown to upregulate IDO in response to HIV infection (Boasso et al., 2007, Blood; 109:3351-3359); thus, authentic human pDCs can be induced to express IDO. Future studies will be needed to address the possible developmental role of IDO and GCN2 in the differentiation of the Treg lineage. Preliminary studies demonstrate selective but significant functional defects in Tregs derived from IDO-KO, GCN2-KO and CHOP-KO mice, suggesting that the IDO pathway may have broader importance for aspects of normal Treg differentiation.

The current study suggests that patients with cancer may have abnormally increased Treg activity in TDLNs, due in part to the effects of IDO. Once tumors are established, simply blocking IDO was not sufficient to fully reverse the suppressive milieu in the TDLN (FIG. 7C). But even in established tumors, blocking IDO allowed initial activation of tumor-specific effector T cells in TDLNs, with attempted cell division. Combining IDO inhibitor drugs with chemotherapy may further help reverse the established suppressive milieu in TDLNs. Therapeutic strategies to block IDO, tumor-induced Tregs, and the PD-1/PD-L pathway are all currently in clinical or pre-clinical development. This demonstration of a molecular link uniting all three of these potent immunosuppressive mechanisms has significant implications for cancer immunotherapy.

The current example demonstrates that immunosuppressive effects occur in two stages. The first stage is the fast activation of pre-existing Tregs by a mechanism that depends on IDO, MHC-matched interaction and GCN2. This stage can be blocked by pharmacological inhibition of IDO with IDO inhibitors. The second stage is the activation of the PD-1/PD-L pathway on DCs, mediated by IDO-activated Tregs. This stage is not dependent on IDO activity but on IDO-dependent-activated Tregs, and can be suppressed by inhibitors of PD-1 and PD-L pathways. The discovery of a sequential mechanistic link between IDO-dependent activation of Tregs and the PD-1/PD-L dependent immunosuppression induced by IDO-activated Tregs indicates that the combination of therapeutic approaches that rely on the combined inhibition of both the IDO pathway and the PD-1/PD-L pathway should have synergistic therapeutic benefits.

### Example 2

### IDO-Activated Tregs Cause Upregulation of PD L1 and PD L2 on Bystander DCs

To address the molecular mechanism of bystander suppression, a model in which the Treg could be activated by IDO in one culture, then re-purified and transferred to a second (“bystander”) culture to mediate suppression was developed. Tregs were activated by culture with IDO plus pDCs, OT I, OVA peptide, and feeder layer, without the bystander cells, as described in Example 1. After two days, the IDO-activated Tregs were resorted based on CD4 expression (which unambiguously identified the Tregs because they were the only CD4⁺ cells in the cultures), and transferred to readout assays comprising A1 cells plus CBA DCs plus HY peptide.

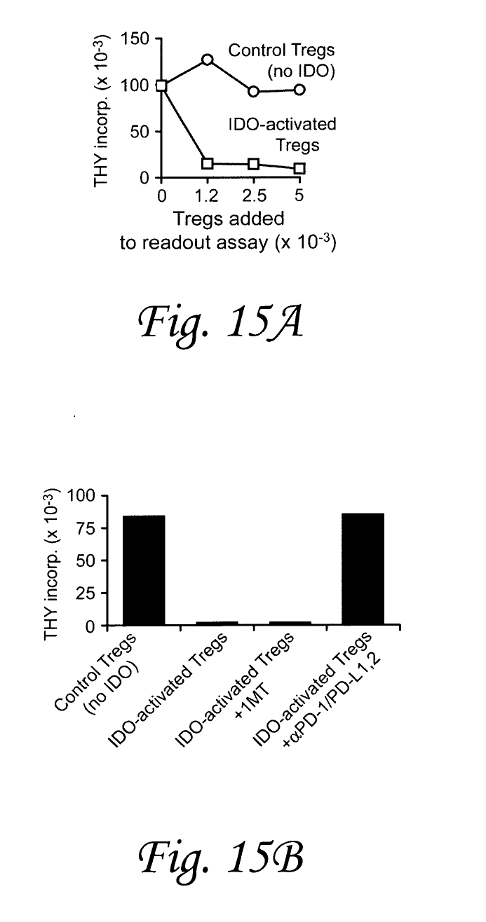

FIG. 15A shows that the IDO-activated Tregs potently suppressed the readout assays; whereas there was minimal suppression by the same Tregs, pre-cultured in the same activation same system, but with IDO blocked by adding 1MT during the pre-activation assay (labeled as the “no IDO” group). The readout assay had no 1MT in any group. Thus, this pre-activation model provided a second, independent method confirming the existence of potent IDO-induced Treg activation, and it allowed the suppressor phase to be studied in isolation from the activation phase.

FIG. 15B uses this activation model to test the effect on Treg-mediated suppression of either 1MT added to the readout assay, or a cocktail of antibodies against the T cell inhibitory receptor PD 1 and its ligands PD L1 and PD L2 (50 μg/ml each). Adding 1MT in the readout assay had no effect on suppression (even though 1MT in the pre-activation assay completely abolished IDO-induced Treg suppressor activity, as shown by the control “no IDO” bar). However, blocking the PD 1/PD ligand system in the readout assay entirely abolished suppression by IDO-activated Tregs. Thus, the mechanism of bystander suppression by the IDO-activated Tregs was independent of IDO, and was mediated by the suppressive PD 1/PD ligand system in the bystander cells.

The role for the PD 1/PD ligand system as a downstream mechanism of IDO-activated Tregs is entirely novel. It occurred only with the IDO-induced form of Treg activation: in other experiments, Tregs activated by conventional means (culture for two days in CD3 plus IL-2 (Thornton et al., 2004, Eur J Immunol; 34:366-76)), showed no effect of PD 1/PD ligand blockade on their form of suppressor activity. Based on these in vitro findings, it is concluded that the PD 1/PD ligand system is activated by IDO in the TDLN, and is the mechanism of IDO-induced bystander suppression in vivo.

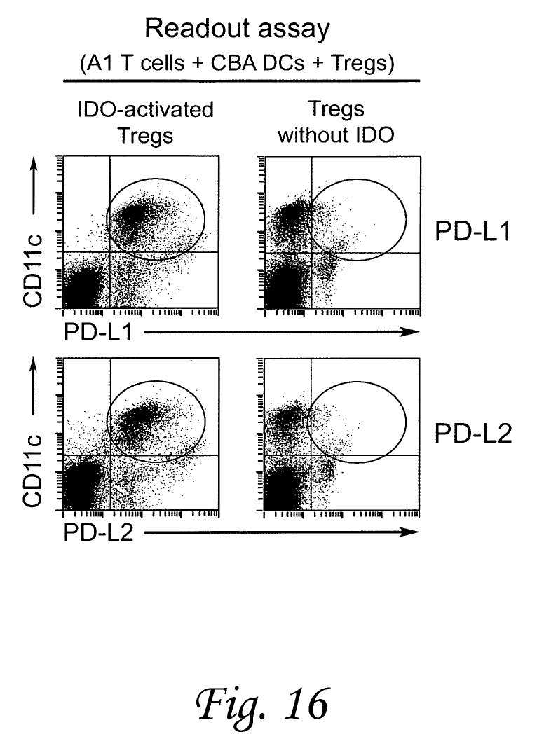

FIG. 16 shows that IDO-pre-activated Tregs strongly upregulated PD L1 and PD L2 expression on the DCs in the readout assay. At the start of culture, these resting, normally non-suppressive DCs showed little detectable expression of PD L1 or PD L2. However, after 48 hours of exposure to IDO-pre-activated Tregs, the DCs had uniformly upregulated PD L1 and PD L2 (circular gates) (PD L1 and PD L2 antibodies were from eBioscience). In contrast, when IDO was blocked during the initial Treg pre activation step (plots labeled “Tregs without IDO”), then there was no upregulation of PD L1/PD L2 on the target DCs. This is consistent with the mechanistic role, found in FIG. 15, for the PD 1/PD ligand system in mediating bystander suppression by IDO-activated Tregs.

### Example 3

### Blocking of PD 1 and Both PD L1 and PD L2 Prevents Suppression by Activated Tregs

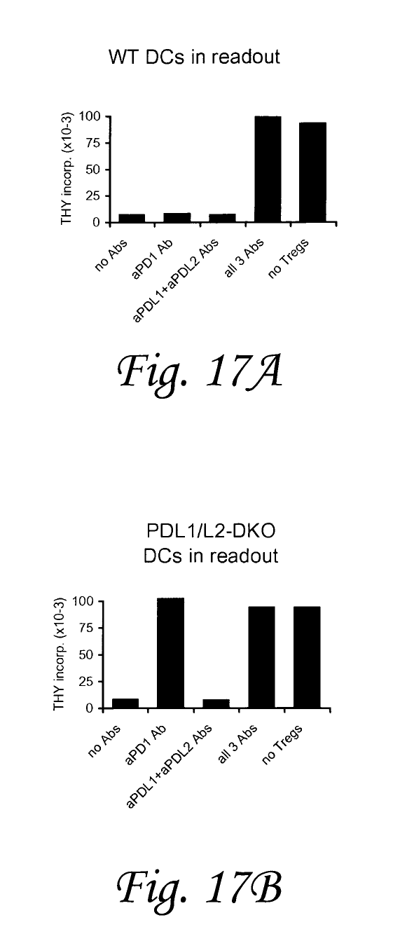

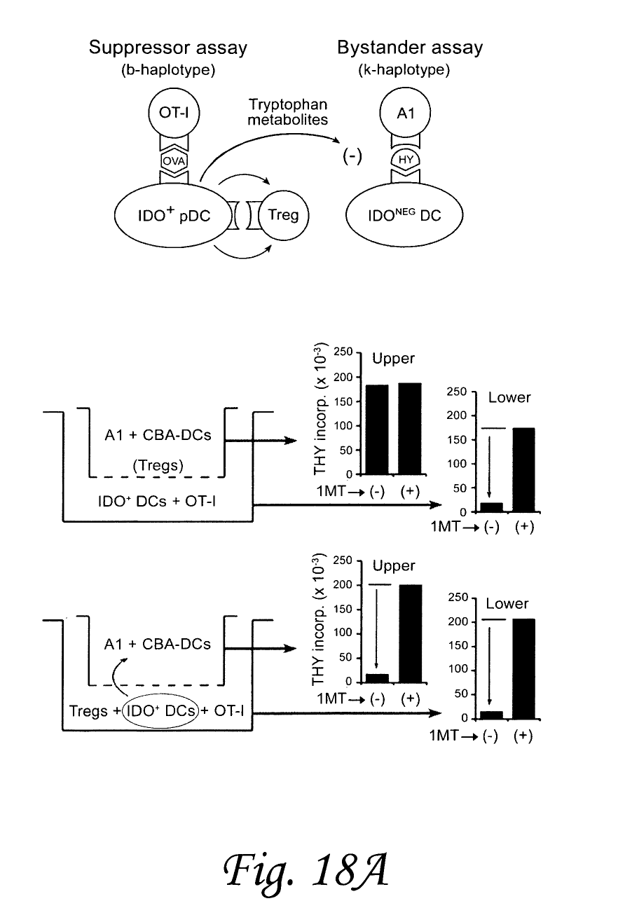

Tregs were activated for two days in co-culture with TDLN pDCs plus OT I plus feeder cells, then harvested, resorted based on CD4 expression, and added to readout assays (A1+CBA DCs plus HY peptide). Readout assays also received either blocking antibodies against PD 1, a mix of antibodies against PD L1 and PD L2, or all 3 antibodies together. Control readout cultures received no Tregs. FIG. 17A shows that only the combination of all three antibodies was able to block suppression mediated by IDO-activated Tregs. Confirming this result, FIG. 17B shows that when the target DCs in the readout assay were genetically deficient in both PD L1 and PD L2 (isolated from PD L1/L2-double-knockout mice) then the PD L1 and PD L2 blocking antibodies were no longer required to reverse suppression (i.e., PD 1 antibody was just as effective as all three antibodies together at reversing suppression), but, importantly, that PD 1 blocking antibody was still required.

The finding that both ligands and the PD 1 receptor must be blocked in order to abrogate the suppressive activity of IDO-activated Tregs implies that PD L1 and PD L2 do not comprise the only relevant ligands for PD 1 (or its related receptors) in this system; and that PD 1 is not the only relevant receptor mediating suppression. This system can therefore be used to identify new and otherwise previously unsuspected receptors and new ligands in the PD 1 and PD ligand families that can mediate suppression by IDO-activated Tregs.

With respect to the clinical use of blocking antibodies against PD 1 and PD ligands, these data suggest that single antibodies alone against PD 1 or PD L1 or PD L2 might not be able to fully block the activity of IDO-activated Tregs in vivo (e.g., in patients with cancer, HIV or chronic viral or bacterial infection). However, a combination of the three targets (PD 1, PD L1 and PD L2) may need to be blocked in vivo in order to achieve the desired therapeutic effect of removing the suppressive activity of IDO-activated Tregs. Moreover, the combination of an IDO-inhibitor drug plus one or more blocking antibodies against the PD 1/PD ligand pathway would be predicted to show synergistic effect, by blocking two different points in the pathway of IDO-induced Treg activation (i.e., the activation step of the Tregs and the effector mechanism by which they suppress).

### Example 4

### Blockade of the PD1/PD L1 Pathway Selectively Prevents Bystander Component Suppression in TDLNs Mediated by IDO-Activated Tregs

In vitro, IDO⁺ pDCs from TDLNs can directly suppress those T cells to which they physically present antigen, but they can also indirectly create potent bystander suppression via IDO-activated Tregs. In particular, the bystander component of suppression has major implications for the biology of the TDLN, because it potentially allows a small number of IDO⁺ pDCs in the TDLN to suppress responses by all T cells, even to antigens presented by other, non-suppressive APCs. But how can the contribution of these two very different mechanisms in real TDLNs be determined? One way of distinguishing between direct suppression (by IDO⁺ pDCs) and indirect suppression (by IDO-activated Tregs) is suggested by the in vitro studies shown in FIG. 15. These demonstrate that bystander suppression mediated by IDO-activated Tregs is strictly dependent on the PD1/PD ligand pathway—if this pathway is blocked, then bystander cells become able to activate despite the presence of IDO-activated Tregs. This Example will determine whether blocking the PD 1/PD ligand system in vivo will selectively remove the indirect bystander (IDO-activated Treg) component of suppression in the TDLN.

B6 mice with B16-OVA tumors will receive oral 1MT (or vehicle) starting on day six, then cyclophosphamide (CY) (or saline) on day seven. On day eight, CFSE-labeled CD8+ OT IThy1.1 T cells will be injected. On days seven and eight mice will receive a PD 1/PD L blocking cocktail comprising anti-PD 1/anti-PD L1/anti-PD L2 antibodies i.v. (100 μg each) or hamster IgG control. Initial in vitro studies suggest that optimal reversal of Treg-mediated bystander suppression may require blocking both PD 1 and PD L1/PD L2 pathways. Thus, to ensure an unambiguous effect in these initial in vivo studies, a cocktail of all three antibodies will be used to start with. Groups will be a) hamster IgG control only (vehicle & saline); b) PD 1/PD L blocking cocktail only (+vehicle & saline); c) 1MT+CY+hamster IgG control; and d) MT+CY+PD 1/PD L blocking cocktail. For readouts, TDLNs will be harvested on day 12 for FACS analysis and sorting.

Treg assay. Suppressor assays will be performed on total TDLN cells. After chemotherapy there are very few Tregs recoverable from TDLNs, but the assay can be performed because it does not require FACS sorting of the Tregs. TDLN cells are titrated in the readout assay in the presence or absence of 1MT, and Treg activity defined as the 1MT-resistant component of suppressor activity. This will be confirmed by using replicate wells receiving both 1MT and PD 1/PD L blocking cocktail in the readout assay (which will block suppression by IDO-activated Tregs in vitro).

Proliferation and activation of OT I in vivo. In parallel experiments, TDLNs will be stained for CFSE, CD8, Thy1.1, and 1B11, and the OT I cells (CD8⁺ Thy1.1⁺) analyzed for cell divisions (CFSE) and 1B11 expression.

It is anticipated that there will be some effect of the PD 1/PD ligand antibody cocktail by itself (second group, above), manifest as upregulation of 1B11 and an increase in the fraction of OT I undergoing cell division by CFSE (compared to the control group, for which these should both be zero). In the groups receiving 1MT+CY, there are two possible outcomes consistent with the hypothesis. If 1MT is perfectly efficient at preventing IDO-induced Treg activation after chemotherapy, then there will already be good responses of the OT I cells, and adding PD 1/PD L antibody cocktail will not further increase the response (since there would be no IDO-activated Tregs in the TDLN); this would be supported in the in vitro Treg assay by a finding of low suppressor activity in both groups receiving 1MT+CY.

However, if 1MT is not complete in its ability to block IDO, then activated Tregs evident in the in vitro Treg assay will be seen, and an enhancing effect of PD 1/PD ligand antibody cocktail in vivo on OT I proliferation (manifest as an increased fraction of OT I undergoing cell division, and increased number of cell divisions per cell). A positive result will be followed up by studies using tumors grown in IDO-KO hosts, which should be resistant to both 1MT and PD 1/PD ligand blockade. Synergistic enhancement of OT I responses by adding PD 1/PD ligand blockade to 1MT+CY, whether due solely or only partially to IDO-induced bystander suppression, would be of therapeutic interest. This combination will be evaluated for its functional anti-tumor efficacy in Example 6.

### Example 5

### Synergistic Effect of PD1/PD L1 Pathway Blockade with Administration of IDO Inhibitors Following Chemotherapy

Blockade of the PD1/PD L1 pathway allows the development of a curative immune response to established tumors when combined with 1MT plus chemotherapy. As shown in FIG. 15, IDO-activated Tregs require the PD1/PD L1 ligand pathway in order to create bystander suppression. This implies that IDO and the PD1/PD ligand system are linked mechanisms for tolerance induction in the TDLN. In addition, both IDO and PD 1/PD ligand system have been suggested to act within the tumor itself to suppress effector function of activated T cells (Uyttenhove et al., 2003, Nat Med; 9:1269-74; Brandacher et al., 2006, Clin Cancer Res; 12:1144-51; Okamoto et al., 2005, Clin Cancer Res; 11:6030-9; and Hirano et al., 2005, Cancer Res; 65:1089-96). Thus, both at the stage of afferent tolerance induction and efferent immune suppression, there is a strong rationale for potential synergy between IDO-inhibitors and agents that target the PD 1/PD ligand pathway.

To ensure the maximal effect in these initial tumor-growth studies, initial studies will be carried out with a cocktail of three anti-PD 1/PD-ligand antibodies. Five×10⁴ B16F10 tumor cells will be implanted subcutaneously on the flank of syngeneic B6 hosts. Treatment groups will be a) vehicle only (control); b) 1MT+CY; c) blocking antibody cocktail (anti-PD L1+anti-PD L2+anti-PD 1, 100 μg each) i.p. on days 8, 12, 15 and 19; and d) 1MT+CY+blocking antibody cocktail. Tumors will be measured over time. Possible readouts include tumor growth, time to 300 mm², and tumor size at day 20 and day 42. Replicate experiments will be performed.

Statistical analysis. The data will be assessed for normality, as well as for other assumptions of ANOVA, and appropriate transformations will be used when necessary. The primary analysis will done on the average growth per day of an individual tumor calculated as the ending tumor size minus the tumor size at day six divided by the number of days of growth. The effect of vaccine on tumor growth will be analyzed using a one-way ANOVA with four treatments (vehicle, 1MT plus CY, blocking antibody, and 1MT plus CY plus blocking antibody). Significant ANOVA results will be compared using a Tukey's adjustment for the multiple comparisons.

Sample size justification. The sample size for this experiment was calculated based on our preliminary data (mean±SD, n=15) of the effect of 1MT and Cy on log tumor size at day 20 (2.42±0.2 vehicle vs. 1.84±0.2 1MT plus CY). It is hypothesized that the blocking antibody group will be no better than the 1MT plus CY group at reducing tumor growth (i.e. mean tumor size for both groups will be 75 mm² at day 20) but that the combination of all three agents will reduce tumor size to 25 mm² at day 20 (i.e. log tumor size of 1.4±0.2). A sample size of 10 mice per group (5 mice per group per experiment at least 2 replicate experiments) provides at least 90% power to detect this difference at alpha=0.01. A positive result would be a statistically significant prolongation of survival, and slower tumor growth, in the group receiving 1MT plus CY plus blocking antibody cocktail, as compared to 1MT plus CY or antibody treatment alone.

A cocktail of antibodies will be used initially to provide the most effective blockade possible, but this multiple blockade may not be necessary, as enhancement of anti-tumor immunotherapy with blockade of either PD 1 alone or PD L1/B7 H1 alone has been observed (Hirano et al., 2005, Cancer Res; 65:1089-96). Therefore, if an effect is seen with the cocktail, individual antibodies will be evaluated alone.

Simply blocking the PD 1/PD ligand system might not provide sufficient positive activating stimulus for a robust anti-tumor response. In that case, addition of vaccine with CpG-ODN adjuvant, will be used to supply the necessary activating stimulus. However, it is already known that 1MT+chemotherapy alone is sufficient to drive development of a significant anti-tumor immune response (Muller et al., 2005, Nat Med; 11:312-9 and Hou et al., 2007, Cancer Res.; 67:792-801). Thus, the most interesting result from this example would be that blocking the PD 1/PD ligand system is able to further significantly enhance this already existing response.

### Example 6

### Tregs Trigger Super-Induction of IDO in pDCs

Tregs trigger production of a soluble suppressive factor. Tregs are known to up-regulate IDO in DCs (Fallarino et al., 2003, Nat. Immunol; 4:1206-1212). This occurs via ligation of B7 molecules on the pDCs by CTLA4 on the Tregs, and the B7 pathway is a potent inducer of IDO in both mice and humans (Fallarino et al., 2003, Nat. Immunol; 4:1206-1212; Mellor et al., 2004, Int. Immunol; 16:1391-1401; and Munn et al., 2004, J. Immunol; 172:4100-4110). IDO, in turn, is known to produce immunosuppressive metabolites of tryptophan (Fallarino et al., 2002, J. Immunol; 176:6752-6761; Terness et al., 2002, J. Exp. Med. 196:447-457; and Frumento et al., 2002, J. Exp. Med; 196:459-468).

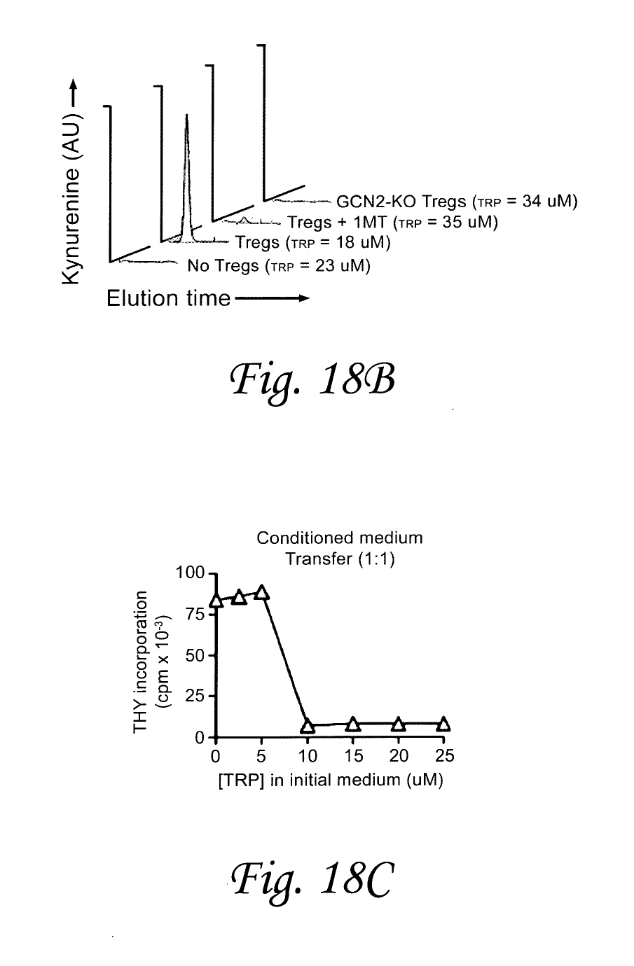

Therefore, this example addressed whether TDLN pDCs produced suppressive tryptophan metabolites in response to Tregs. Bystander assays were performed in transwell inserts, with the bystander cells separated from the TDLN pDCs by a microporous membrane (FIG. 18A). The feeder cells could be placed in either chamber with identical results; in the studies shown in FIG. 18A the feeder cells were in the lower chamber. Bar graphs show [³H]thymidine incorporation, measured separately in each chamber, with or without 1MT added to both chambers. The Tregs were either placed in the lower chamber along with the IDO⁺ pDCs, or in the upper chamber where they could not contact the IDO⁺ pDCs, and thus could not activate IDO (identical results were also obtained by omitting the Tregs altogether). When the Tregs were not in contact with the IDO⁺ pDCs (upper panel of FIG. 18A), the pDCs suppressed only those cells with which they were in direct physical contact (the OT-I cells in the lower well), while the A1 cells in the upper well were unaffected (shown by [³H]thymidine incorporation measured separately in each chamber). However, when the Tregs were placed in contact with the IDO⁺ pDCs, then proliferation in both upper and lower chambers was suppressed, in a 1MT-reversible fashion. Thus, IDO appeared to function at two levels of activity: a basal level, triggered by the OT-I cells and capable only of direct, contact-mediated suppression; and a “super-induced” level, triggered by Tregs and capable of long-distance suppression via a soluble factor. This soluble factor was different from the mechanism of suppression of the activated Tregs, which required cell-cell contact; however, the soluble factor was induced by Tregs, and (as shown below) was a key participant in IDO-mediated Treg activation.

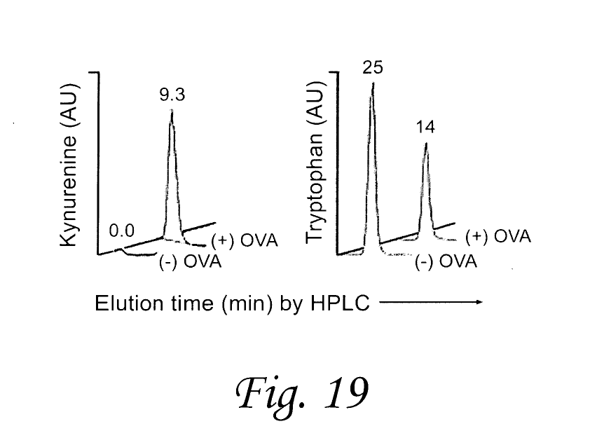

Activated Tregs super-induce IDO activity. In FIG. 18B supernatants from bystander assays, with or without Tregs, were analyzed by HPLC for kynurenine (Munn et al., 2004, J Immunol; 172:4100-4110). Cultures for HPLC analysis contained five times the usual number of pDCs. HPLC analysis of supernatants from bystander cultures showed that kynurenine (the first major metabolite of tryptophan produced by IDO) accumulated at higher levels in cultures containing Tregs than in those without Tregs (FIG. 19B). Cultures without Tregs still showed detectable depletion of tryptophan from the medium, and this was blocked by 1MT, indicating that IDO was enzymatically active. In the absence of Tregs kynurenine accumulation in the medium was low, but kynurenine can be rapidly converted into other breakdown products (Belladonna et al., 2006, J Immunol; 177:130-137), so kynurenine is only one proxy for overall IDO activity. Thus, in the absence of Tregs, IDO functioned at a lower (basal) level, and the addition of Tregs cause super-induction of IDO activity. With or without Tregs, it was still necessary for pDCs to present antigen to OT-I cells in order to trigger functional IDO, as shown in FIG. 19. FIG. 18B also shows that Tregs from GCN2-KO mice, which were unable to respond to IDO, were also unable to trigger super-induction of tryptophan catabolism. Thus, the super-induction of IDO by Treg was a secondary event, downstream of the initial GCN2-dependent activation of the Tregs by IDO, in a self-amplifying paracrine system.

FIG. 19 demonstrates that antigen presentation to OT-I cells is required to trigger functional IDO enzyme activity. IDO activity was measured as tryptophan depletion and kynurenine production in culture supernatants. Bystander-suppression assays were set up containing all of the cell populations, including the Tregs. Assays were performed with and without the cognate OVA peptide (SIINFEKL) (SEQ ID NO:1) to activate the OT-I cells. Both assays received the H-Y antigen for the A1 cells. Supernatants were harvested after 72 hours and analyzed by HPLC as described (Munn et al., 2004, J Immunol; 172:4100-4110). The HPLC traces show the kynurenine and tryptophan peaks for groups with and without OVA. The concentration (in μM) of tryptophan and kynurenine in the medium is shown above each peak, interpolated from a standard curve. IDO only became enzymatically active (produced kynurenine and depleted tryptophan) when the pDCs presented antigen to OT-I, even though Tregs and all other cells were present in both groups.

Generation of metabolites is prevented by low-tryptophan medium. To determine if the soluble suppressor factor in FIG. 18A was a metabolite of tryptophan, it was addressed whether the factor could no longer be produced if the initial concentration of tryptophan in the medium was made artificially low. Since each metabolite is made in a 1:1 stoichiometry from the preceding one, all metabolite production is strictly limited by the initial supply of tryptophan. Cultures were set up containing TDLN pDCs+Tregs+OT-I+feeder cells, with various concentrations of tryptophan in the medium. After 18 hours, the conditioned medium was harvested and transferred to readout assays containing A1 T cells+CBA DCs. All readout assays contained a 1:1 dilution of fresh medium, so there was always ample tryptophan to support T cell proliferation, irrespective of the tryptophan in the conditioned medium. As shown in FIG. 18C, those cultures initially containing less than 10 μM tryptophan were unable to generate any detectable soluble suppressor factor in their conditioned media, consistent with the soluble factor being a metabolite of tryptophan.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

### Sequence Listing Free Text

| | |
|---|---|
| SEQ ID NO: 1 | ovalbumin peptide |
| SEQ ID NO: 2 | H-Y peptide |

1. A method of enhancing an immune response comprising administering an inhibitor of indoleamine-2,3-dioxygenase (IDO) and one or more inhibitors of the PD-1/PD-L pathway.

2. A method to enhance an immune response to an antigen in a subject, the method comprising administering to the subject an effective amount of such an antigen in combination with an inhibitor of DO and one or more inhibitors of the PD-1/PD-L pathway.

3. A method of reducing immune suppression mediated by regulatory T cells (Tregs) in a subject, the method comprising administering to the subject an inhibitor of indoleamine-2,3-dioxygenase (IDO) and one or more inhibitors of the PD-1/PD-L pathway.

4. A method of enhancing a T cell mediated immune response, the method comprising administering the method comprising administering to the subject an inhibitor of indoleamine-2,3-dioxygenase (IDO) and one or more inhibitors of the PD-1/PD-L pathway.

5. A method of treating cancer in a subject, the method comprising administering to the subject an inhibitor of indoleamine-2,3-dioxygenase (IDO) and one or more inhibitors of the PD-1/PD-L pathway.

6. A method of treating a subject with an infection, the method comprising administering to the subject an inhibitor of indoleamine-2,3-dioxygenase (IDO) and one or more inhibitors of the PD-1/PD-L pathway.

7. The method of claim 1, comprising the administration of two or more inhibitors of the PD-1/PD-L pathway.

8. The method of claim 7 wherein the two or more inhibitors of the PD-1/PD-L pathway are administered in combination, as a cocktail.

9. The method of claim 1, where one or more inhibitors of the PD-1/PD-L pathway comprise one or more antibodies against PD-1, PD-L1, and/or PD-L2.

10. The method of claim 1 further comprising the administration of one or more inhibitors of the CTLA4 pathway.

11-16. (canceled)

17. The method of claim 10, where the inhibitors of the CTLA4 pathway comprise one or more antibodies against CTLA4.

18. (canceled)

19. The method of claim 1 further comprising the administration of an additional therapeutic agent.

20. The method of claim 19 wherein the additional therapeutic agent is a cytotoxic chemotherapeutic agent.

21. The method of claim 2 wherein the antigen is a tumor antigen.

22. The method of claim 21 wherein the tumor antigen is delivered as a vaccine, a recombinant viral vector, or autologous or allogeneic tumor cells or cell line.

23. (canceled)

24. The method of claim 1, wherein the inhibitor of IDO is 1-methyl-tryptophan (1-MT).

25. The method of claim 24, where 1-MT is selected from the group consisting of an isolated D isomer of 1-MT, an isolated L isomer of 1-MT, and a racemic mixture of 1-MT.

26. The method of claim 19 wherein the additional therapeutic agent is an antibody to IL-10.

27. The method of claim 19 wherein the additional therapeutic agent is an antibody to TGF-β.
